SEQUENCE LISTING (10 sequences)

SEQ ID NO: 1 - DNA, 17 bp, unidentified
  source 1..17: mol_type = unassigned DNA
gagcgcgctggacttct

SEQ ID NO: 2 - DNA, 17 bp, unidentified
  source 1..17: mol_type = unassigned DNA
gacttctaatccagagg

SEQ ID NO: 3 - RNA, 58 nt, unidentified
  source 1..58: mol_type = other RNA
ccggaagagcgcgctggacttctaactcgagttagaagtccagcgcgctctttttttg

SEQ ID NO: 4 - RNA, 58 nt, unidentified
  source 1..58: mol_type = other RNA
ccggaagacttctaatccagaggttctcgagaacctctggattagaagtctttttttg

SEQ ID NO: 5 - DNA, 16 bp, synthetic construct
  source 1..16: mol_type = other DNA
  modified_base 1: mod_base = OTHER; note = Locked nucleic acid
  modified_base 2: mod_base = OTHER; note = Locked nucleic acid
  modified_base 3: mod_base = OTHER; note = Locked nucleic acid
  modified_base 14: mod_base = OTHER; note = Locked nucleic acid
  modified_base 15: mod_base = OTHER; note = Locked nucleic acid
  modified_base 16: mod_base = OTHER; note = Locked nucleic acid
agcgcgctcgtccatt

SEQ ID NO: 6 - DNA, 16 bp, synthetic construct
  source 1..16: mol_type = other DNA
  modified_base 1: mod_base = OTHER; note = Locked nucleic acid
  modified_base 2: mod_base = OTHER; note = Locked nucleic acid
  misc_feature 3
  modified_base 14: mod_base = OTHER; note = Locked nucleic acid
  modified_base 15: mod_base = OTHER; note = Locked nucleic acid
  modified_base 16: mod_base = OTHER; note = Locked nucleic acid
tctctgccgggactcg

SEQ ID NO: 7 - DNA, 16 bp, synthetic construct
  source 1..16: mol_type = other DNA
  modified_base 1: mod_base = OTHER; note = Locked nucleic acid
  modified_base 2: mod_base = OTHER; note = Locked nucleic acid
  modified_base 3: mod_base = OTHER; note = Locked nucleic acid
  modified_base 14: mod_base = OTHER; note = Locked nucleic acid
  modified_base 15: mod_base = OTHER; note = Locked nucleic acid
  modified_base 16: mod_base = OTHER; note = Locked nucleic acid
aagtccagcgcgctcg

SEQ ID NO: 8 - protein, 29 aa, synthetic construct
AALEALAEALEALAEALEALAEAAAAGGC

SEQ ID NO: 9 - protein, 30 aa, synthetic construct
AALAEALAEALAEALAEALAEALAAAAGGC

SEQ ID NO: 10 - protein, 15 aa, synthetic construct
ALEALAEALEALAEA

(19) United States
(12) Patent Application Publication
(10) Pub. No.: US 2026/0139250 A1
(43) Pub. Date: May 21, 2026

(54) METHODS AND COMPOSITIONS FOR THE TREATMENT OF CANCER BY TARGETING ONCOGENIC TRANSFER RNAS

(71) Applicant: THE CHILDREN'S MEDICAL CENTER CORPORATION, Boston, MA (US)

(72) Inventors: Richard I. GREGORY, Brookline, MA (US); Esteban A. ORELLANA, Brookline, MA (US)

(21) Appl. No.: 18/881,165

(22) PCT Filed: Jul. 5, 2023
(86) PCT No.: PCT/US2023/069629

§ 371 Date: Jan. 3, 2025

Related U.S. Application Data
(60) Provisional application No. 63/358,280, filed on Jul. 5, 2022.

(51) Int. Cl. C12N 15/113; A61K 31/712; A61K 47/69; A61P 35/00; C12N 15/88

(52) U.S. Cl. CPC C12N 15/113; A61K 31/712; A61K 47/6911; A61P 35/00; C12N 15/88; C12N 2310/113; C12N 2310/14; C12N 2310/3231; C12N 2310/531

(57) ABSTRACT

Provided herein are methods and compositions for reducing the expression and/or activity of an oncogenic transfer RNA (tRNA). Composition for treating cancer in the subject are disclosed, comprising an inhibitor of an oncogenic transfer RNA (tRNA) to a subject in need thereof, wherein the oncogenic tRNA comprises an inhibitory nucleic acid such as ARG-TCT-4-1, an siRNA, an shRNA, an miRNA, an antisense oligonucleotide, or a nucleic acid modification such as a locked nucleic acid, a phosphorothioate modification, a 2′—O-methyl modification, a 2′—O-methoxyethyl modification, a 2′-fluoro modification, a phosphorodiamidate modification, or a mesylphosphoramidate modification. The disclosed compositions are disclosed as treating cancers such as a sarcoma, a glioblastoma, an adrenocortical carcinoma, a cholangiocarcinonma, a melanoma, a glioma, a diffuse glioma, a mature B cell neoplasm, a non-small cell lung cancer, an esophagogastric adenocarcinoma, a pheochromocytoma, a hepatocellular carcinoma, an endometrial carcinoma, a pancreatic adenocarcinoma, a breast carcinoma, an invasive breast carcinoma, a head and neck squamous cell carcinoma, a bladder urothelial carcinoma, a colorectal adenocarcinoma, an ovarian epithelial tumor, a prostate adenocarcinoma, a cervical squamous cell carcinoma, a renal non-clear cell carcinoma, or a renal clear cell carcinoma.

### CROSS REFERENCE PARAGRAPH TO RELATED APPLICATIONS

This application claims benefit under 35 U.S.C. § 119(e) of U.S. Provisional Application No. 63/358,280, filed Jul. 5, 2022, the contents of which are incorporated herein by reference in its entirety.

### GOVERNMENT SUPPORT

This invention was made with Government support under grant no. R35CA232115 awarded by the National Institutes of Health. The Government has certain rights in this invention.

### SEQUENCE LISTING

The instant application contains a Sequence Listing that has been submitted electronically in XML format via Patent Center and is hereby incorporated by reference in its entirety. Said XML copy, created on Nov. 19, 2025, is named “025630-000101USPX_SL.xml” and is 19,323 bytes in size.

### TECHNICAL FIELD

The technology described herein relates to methods and compositions for inhibiting an oncogenic transfer RNA (tRNA).

### BACKGROUND

There is evidence linking dysregulation of individual tRNAs with disease. Overexpression of tRNAi-Met leads to increased metabolic and cell growth rates in immortalized human breast cells, promotes melanoma metastasis, and increases tumor growth and vascularization in mice. Overexpression of tRNA-Glu(UCC) or tRNA-Arg(CCG) promotes a pro-metastatic state in breast cancer. Furthermore, deficiency of the Arg-TCT-4-1 isodecoder that is highly expressed in the central nervous system (CNS) causes neurodegeneration and death in mice.

### SUMMARY

The methods and compositions described herein are based, in part, on the discovery that overexpression of methyltransferase-like protein 1 (METTL1) induces oncogenesis of a variety of cancer types, including liposarcoma. Further, it was discovered that overexpression of METTL1 increases the expression of the oncogenic transfer RNA, ARG-TCT-4-1. Accordingly, provided herein are methods and compositions for inhibiting oncogenesis or treating cancer comprising methods and compositions that target ARG-TCT-4-1 activity and/or expression.

One aspect provided herein relates to a method for treating cancer, the method comprising administering a composition comprising an inhibitor of an oncogenic transfer RNA (tRNA) to a subject in need thereof, wherein the oncogenic tRNA comprises ARG-TCT-4-1, thereby treating cancer in the subject.

In one embodiment of this aspect and all other aspects provided herein, the inhibitor reduces expression and/or activity of the oncogenic tRNA.

In another embodiment of this aspect and all other aspects provided herein, the inhibitor sequesters the oncogenic tRNA, thereby reducing activity of the oncogenic tRNA.

In another embodiment of this aspect and all other aspects provided herein, the inhibitor comprises an inhibitory nucleic acid.

In another embodiment of this aspect and all other aspects provided herein, the inhibitory nucleic acid comprises a targeting sequence complementary to a sequence of ARG-TCT-4-1 of about 15-25 nucleotides in length.

In another embodiment of this aspect and all other aspects provided herein, the targeting sequence comprises SEQ ID NO: 1, or SEQ ID NO: 2.

In another embodiment of this aspect and all other aspects provided herein, the inhibitory nucleic acid is about 25-65 nucleotides in length.

In another embodiment of this aspect and all other aspects provided herein, the inhibitory nucleic acid comprises an siRNA, an shRNA, an miRNA, or an antisense oligonucleotide.

In another embodiment of this aspect and all other aspects provided herein, the inhibitory nucleic acid is an shRNA and comprises a sequence of SEQ ID NO: 3 or SEQ ID NO: 4.

In another embodiment of this aspect and all other aspects provided herein, the inhibitory nucleic acid is an antisense oligonucleotide and comprises a sequence of SEQ ID NOs: 5, 6 or 7.

In another embodiment of this aspect and all other aspects provided herein, the inhibitory nucleic acid comprises a nucleic acid modification.

In another embodiment of this aspect and all other aspects provided herein, the nucleic acid modification comprises a locked nucleic acid, a phosphorothioate modification, a 2′-O-methyl modification, a 2′-O-methoxyethyl modification, a 2′-fluoro modification, a phosphorodiamidate modification or a mesylphosphoramidate modification.

In another embodiment of this aspect and all other aspects provided herein, the inhibitory nucleic acid specifically binds ARG-TCT-4-1.

In another embodiment of this aspect and all other aspects provided herein, the inhibitory nucleic acid binds ARG-TCT-4-1 at a concentration that is at least 2-fold lower than the concentration of the inhibitory nucleic acid that binds an off-target tRNA

In another embodiment of this aspect and all other aspects provided herein, the inhibitory nucleic acid does not bind to off-target tRNA.

In another embodiment of this aspect and all other aspects provided herein, the cancer comprises increased expression of ARG-TCT-4-1 and/or methyltransferase-like 1 protein (METTL1).

In another embodiment of this aspect and all other aspects provided herein, the subject is diagnosed as having a cancer to be treated as described herein by detecting an increase in the levels of ARG-TCT-4-1 and/or METTL1 in a biological sample obtained from the subject (e.g., a biological sample comprising at least one cancer cell, such as a tumor or tissue biopsy or blood sample).

In another embodiment of this aspect and all other aspects provided herein, the cancer is a sarcoma, a glioblastoma, an adrenocortical carcinoma, a cholangiocarcinoma, a melanoma, a glioma, a diffuse glioma, a mature B cell neoplasm, a non-small cell lung cancer, an esophagogastric adenocarcinoma, a pheochromocytoma, a hepatocellular carcinoma, an endometrial carcinoma, a pancreatic adenocarcinoma, a breast carcinoma, an invasive breast carcinoma, a head and neck squamous cell carcinoma, a bladder urothelial carcinoma, a colorectal adenocarcinoma, an ovarian epithelial tumor, a prostate adenocarcinoma, a cervical squamous cell carcinoma, a renal non-clear cell carcinoma, or a renal clear cell carcinoma.

In another embodiment of this aspect and all other aspects provided herein, the sarcoma is a liposarcoma.

In another embodiment of this aspect and all other aspects provided herein, the composition further comprises a lipid composition or a lipid nanoparticle.

In another embodiment of this aspect and all other aspects provided herein, the inhibitory nucleic acid, the lipid composition or the lipid nanoparticle further comprises a targeting moiety.

In another embodiment of this aspect and all other aspects provided herein, the targeting moiety comprises an antigen or antigen-binding fragment thereof that binds to a cancer cell marker.

In another embodiment of this aspect and all other aspects provided herein, the targeting moiety comprises a ligand.

Another aspect provided herein relates to a composition comprising an inhibitor of ARG-TCT-4-1 and a pharmaceutically acceptable carrier.

In one embodiment of this aspect and all other aspects provided herein, the inhibitor of ARG-TCT-4-1 comprises an inhibitory nucleic acid.

In another embodiment of this aspect and all other aspects provided herein, the inhibitory nucleic acid comprises a targeting sequence complementary to a sequence of ARG-TCT-4-1 of about 15-25 nucleotides in length.

In another embodiment of this aspect and all other aspects provided herein, the targeting sequence comprises SEQ ID NO: 1, or SEQ ID NO: 2.

In another embodiment of this aspect and all other aspects provided herein, the inhibitory nucleic acid is about 25-65 nucleotides in length.

In another embodiment of this aspect and all other aspects provided herein, the inhibitory nucleic acid comprises an siRNA, an shRNA, an miRNA, or an antisense oligonucleotide.

In another embodiment of this aspect and all other aspects provided herein, the inhibitory nucleic acid is an shRNA and comprises a sequence of SEQ ID NO: 3 or SEQ ID NO: 4.

In another embodiment of this aspect and all other aspects provided herein, the inhibitory nucleic acid is an antisense oligonucleotide and comprises a sequence of SEQ ID NOs: 5, 6 or 7.

In another embodiment of this aspect and all other aspects provided herein, the inhibitory nucleic acid comprises a nucleic acid modification.

In another embodiment of this aspect and all other aspects provided herein, the nucleic acid modification comprises a locked nucleic acid, a phosphorothioate modification, a 2′-O-methyl modification, a 2′-O-methoxyethyl modification, a 2′-fluoro modification, a phosphorodiamidate modification or a mesylphosphoramidate modification.

In another embodiment of this aspect and all other aspects provided herein, the inhibitory nucleic acid specifically binds ARG-TCT-4-1.

In another embodiment of this aspect and all other aspects provided herein, the inhibitory nucleic acid binds ARG-TCT-4-1 at a concentration that is at least 2-fold lower than the concentration of the inhibitory nucleic acid that binds an off-target tRNA.

In another embodiment of this aspect and all other aspects provided herein, the inhibitory nucleic acid does not bind to off-target tRNA.

In another embodiment of this aspect and all other aspects provided herein, the composition further comprises a lipid composition or a lipid nanoparticle. Alternatively, the inhibitory nucleic acid is administered naked.

In another embodiment of this aspect and all other aspects provided herein, the inhibitory nucleic acid, the lipid composition or the lipid nanoparticle further comprises a targeting moiety.

In another embodiment of this aspect and all other aspects provided herein, the targeting moiety comprises an antigen or antigen-binding fragment thereof that binds to a cancer cell marker.

In another embodiment of this aspect and all other aspects provided herein, the targeting moiety comprises a ligand.

Another aspect provided herein relates to a method for sequestering ARG-TCT-4-1 in a cell, the method comprising contacting a cell expressing ARG-TCT-4-1 with an inhibitory nucleic acid, wherein the inhibitory nucleic acid binds to and sequesters the ARG-TCT-4-1 tRNA, thereby reducing activity of the ARG-TCT-4-1 tRNA.

In one embodiment of this aspect and all other aspects provided herein, the expression of the ARG-TCT-4-1 tRNA is not altered.

In another embodiment of this aspect and all other aspects provided herein, the inhibitory nucleic acid comprises a targeting sequence complementary to a sequence of ARG-TCT-4-1 of about 15-25 nucleotides in length.

In another embodiment of this aspect and all other aspects provided herein, the targeting sequence comprises SEQ ID NO: 1, or SEQ ID NO: 2.

In another embodiment of this aspect and all other aspects provided herein, the inhibitory nucleic acid is about 25-65 nucleotides in length.

In another embodiment of this aspect and all other aspects provided herein, the inhibitory nucleic acid comprises an siRNA, an shRNA, an miRNA, or an antisense oligonucleotide.

In another embodiment of this aspect and all other aspects provided herein, the inhibitory nucleic acid is an shRNA and comprises a sequence of SEQ ID NO: 3 or SEQ ID NO: 4.

In another embodiment of this aspect and all other aspects provided herein, the inhibitory nucleic acid is an antisense oligonucleotide and comprises a sequence of SEQ ID NOs: 5, 6 or 7.

In another embodiment of this aspect and all other aspects provided herein, the inhibitory nucleic acid comprises a nucleic acid modification.

In another embodiment of this aspect and all other aspects provided herein, the nucleic acid modification comprises a locked nucleic acid, a phosphorothioate modification, a 2′-O-methyl modification, a 2′-O-methoxyethyl modification, a 2′-fluoro modification, a phosphorodiamidate modification or a mesylphosphoramidate modification.

In another embodiment of this aspect and all other aspects provided herein, the inhibitory nucleic acid specifically binds ARG-TCT-4-1.

In another embodiment of this aspect and all other aspects provided herein, the inhibitory nucleic acid binds ARG-TCT-4-1 at a concentration that is at least 2-fold lower than the concentration of the inhibitory nucleic acid that binds an off-target tRNA.

In another embodiment of this aspect and all other aspects provided herein, the inhibitory nucleic acid does not bind to off-target tRNA.

In another embodiment of this aspect and all other aspects provided herein, the inhibitory nucleic acid is administered with a lipid composition. Alternatively, the inhibitory nucleic acid is administered naked (e.g., a naked antisense oligonucleotide (ASO)).

In another embodiment of this aspect and all other aspects provided herein, the inhibitory nucleic acid is in or on a lipid nanoparticle.

In another embodiment of this aspect and all other aspects provided herein, the inhibitory nucleic acid, the lipid composition or the lipid nanoparticle further comprises a targeting moiety.

In another embodiment of this aspect and all other aspects provided herein, the targeting moiety comprises an antigen or antigen-binding fragment thereof that binds to a cancer cell marker.

In another embodiment of this aspect and all other aspects provided herein, the targeting moiety comprises a ligand.

### BRIEF DESCRIPTION OF THE DRAWINGS

FIGS. 1A-1G. FIG. 1A demonstrates competitive co-culture of lentiviral METTL or WDR4 or empty gRNA-transfected (BFP positive) versus untransfected panel of human AML cell lines normalized to % BFP on day 4 (mean±SD, n=2). FIG. 1B shows bioluminescence imaging of mice transplanted with luciferase-expressing MOLM-13 cells, at the indicated time points, upon transduction with METTL1 or control gRNAs. Quantification of bioluminescence and Kaplan-Meier plot showing the mouse survival. A log rank test was performed (n=5 animals per groups). **p<0.01. FIG. 1C examines the BrdU staining and in vivo cell cycle analysis in gRNA transduced MOLM-13 cells at day 10 post-transplantation. FIG. 1D depicts a western blot showing METTL1 ectopic expression in stable METTL1-knockdown (KD) human GBM cells (LNZ308). FIG. 1E examines cell proliferation analysis of KD cells expressing wild-type or mutant METTL1. Error bars denote mean±SD. Each experiment corresponds to n=3; each experiment was repeated three times. ****p<0.0001; ns, not significant. Two-way analysis of variance (ANOVA) and Bonferroni correction. FIG. 1F depicts cell cycle analysis of LNZ308 cells comparing sh-METTL1 versus shGFP control. FIG. 1G examines in vivo tumor formation of LNZ308 (n=5; error bars denote mean±SEM). ****p<0.0001. Two-way analysis of variance (ANOVA) and Bonferroni correction.

FIGS. 2A-2F. FIG. 2A shows a subset of m⁷G-modified tRNAs identified in human GBM cells LNZ308. FIG. 2B demonstrates the changes in tRNA abundance upon METTL2 knockdown. FIG. 2C examines tRNA levels measured via northern blot. FIG. 2D depicts HPLC-MS/MS analysis of total RNA comparing sh-METTL1 samples versus shGFP control samples. Error bars denotes mean±SD. Each experiment corresponds to n=3 and the p value from paired Student's t test. FIG. 2E examines [35S]methionine incorporation measured via liquid scintillation where CPM is counts per minutes and n=3, measured by a paired Student's t test. **p<0.01. FIG. 2F exhibits changes in protein abundance between METTL1/WDR4 (heavy) overexpressing cells and empty vector (light) control cells measured by SILAC-based proteomics; n=3, moderated t test.

FIGS. 3A-3I. FIG. 3A depicts a proliferation assay using MOLM-13 and THP-1 cells after overexpression of METTL1 (WT and catalytic-dead) and compared with empty control (mean±SD, n=3). *p<0.05. FIG. 3B shows a competitive co-culture of human AML MOLM-13-dCas9 cells post transduction with either METTL1 or empty gRNA (BFP positive) versus untransfected panel, normalized to % BFP on day 4 (mean±SD, n=2). FIG. 3C examines a colony formation assay of primary non-leukemic Nras^{G12D/+} lineage-negative HSPCs, upon ectopic expression of WT and catalytic mutant METTL1 compared with empty control. CFU, colony forming units; ns, not significant (mean±SD, n=3). *p<0.01 (t test). FIG. 3D demonstrates representative cell proliferation analysis of METTL1-WT/WDR4-overexpressing cells compared with METTL1-Mut/WDR4 and empty vector control cells. Error bars denote mean±SD. Each experiment corresponds to n=3; each experiment was repeated three times. ****p<0.0001; ns, not significant. Two-way analysis of variance (ANOVA) and Bonferroni correction. FIG. 3E examines colony formation in soft agar which are representative figures. FIG. 3F analyzes the quantification of colony formation in soft agar. Error bars denote mean±SD. Each experiment corresponds to n=3; each experiment was repeated three times. ****p<0.0001; ns, not significant. One-way analysis of variance (ANOVA) and Bonferroni correction. FIG. 3G shows cell cycle analysis of METTL1/WDR4-overexpressing cells versus empty control. DNA content (2N, >2N, or 4N) was analyzed at different time points after BrdU labeling. Bars indicate the percentage of BrdU+ cells (0 h) that transitioned from 2N DNA content to 4N DNA (G2) and after undergoing mitosis transitioned to 2N DNA. FIG. 3H examines DNA content of BrdU+ cells of METTL1/WDR4-overexpressing cells versus empty vector control at 0 and 6 h post-labeling. FIG. 3I demonstrates in vivo tumor formation (n=5; error bars denote mean±SEM). Error bars denote mean±SD. *p<0.05; ns, not significant. One-way analysis of variance (ANOVA) and Bonferroni correction.

FIGS. 4A-4G. FIG. 4A depicts a subset of m⁷G-modified tRNA identified in mouse MEF-WT cells. FIG. 4B shows an overlap of m⁷G tRNAs among different conditions. FIG. 4C examines changes in tRNA abundance upon overexpression of METTL1-WtWDR4. On the right, Arg-TCT-4 levels measured via northern blot. FIG. 4D analyzes HPLC-MS/MS of total RNA comparing METTL1/WDR4-overexpressing samples versus empty vector control samples. Error bars denote mean±DS. Each experiment corresponds to n=3. P value from paired Student's t test. FIG. 4E examines a correlation between tRNA abundance change and change in m⁷G methylation status measured as a change in NaBH₄/aniline cleaved tRNA fragments in METTL1-WT/WDR4 versus empty vector and used the Pearson correlation. FIG. 4F analyzes HPLC-MS/MS of isolated Arg-TCT tRNA comparing METTL1/WDR4-overexpressing samples versus empty vector control samples. Oligo 1: Arg-TCT-1,2,3,5; oligo 2: Arg-TCT-4. Error bars denote mean±SD. Each experiment corresponds to n=2. P value from paired Student's t test. FIG. 4G shows the quantification of m⁷G levels in Arg-TCT-4-1 using time-dependent NaBH₄/aniline cleavage followed by northern blot in empty vector and METTL1/WDR4-overexpressing (OE) samples (ratio between 3′ fragment and full length).

FIGS. 5A-5H. FIG. 5A depicts a scatterplot of translation efficiency (TE) in METTL1-WT/WDR4-OE versus empty vector cells. TE was calculated by dividing the ribosome-protected fragment (RPF) signals by the input RNA-seq signals. FIG. 5B examines the ribosome occupancy at individual codons at A sites and A+1 sites. Plots represent the relative ribosome protected fragment signals from METTL1/WDR4 relative to empty vector control cells. The codons are separated into m⁷G (red) and not m⁷G-modified (black) groups. The codons in red correspond to the group of codons with corresponding tRNAs increased in abundance upon METTL1/WDR4 overexpression. Dots in pink represent codons decoded by m⁷G tRNAs by wobble effect due to the undetected levels of their corresponding tRNAs. FIG. 5C shows overall codon occupancy among the groups of codons with corresponding tRNAs increased in abundance (Up), other m⁷G decode codons whose tRNAs do not show changes in abundance (Non) and non-m⁷G-dependent codons (Other). P values from one-way ANOVA (mean±SD). *p<0.05; ns, not significant. FIG. 5D analyzes a Pearson correlation between A site occupancy and tRNA abundance changes. FIG. 5E examines a scatterplot of codon use changes in the differentially translated genes (up versus down and up versus all other) in METTL1/WDR4-OE cells. Dot in red indicate m⁷G-decoded codons. On the right, comparison of AGA codon use between TE-up genes versus all other genes. P value from Student's t test (mean±SD). FIG. 5F examines ribosome pausing in AGA codons between empty vector and METTL1/WDR4-OE cells. P value was calculated using a two-sided Mann-Whitney test (mean±SD). FIG. 5G shows a gene ontology analysis of Reactome pathway enrichment using the TE downregulated and upregulated genes upon METTL1-WT/WDR4 overexpression. FIG. 5H depicts a scatterplot of codon use changes in upregulated (FC>1.2) proteins in METTL1-WT/WDR4-OE cells (up versus down and up versus all other) in METTL1/WDR4-OE cells. Dots in red indicate m⁷G-decoded codons. On the right, comparison of AGA codon use between upregulated proteins versus down and non-change. P value from one-way ANOVA (mean±SD). *p<0.05 and ***p<0.001.

FIGS. 6A-6M. FIG. 6A shows altered tRNA (m⁷G subset) expression in human tumors compared with normal counterparts. Orange denotes upregulation and blue downregulation. Boxplot on right summarizes tumor types with up- or downregulation. FIG. 6B analyzes a Pearson correlation between METTL1 and Arg-TCT expression levels in 22 human tumors. FIG. 6C examines a Kaplan-Meier survival curve of SARC patients with low versus high Arg-TCT expression levels. Mean cut-off. Data are from TCGA. Wilcoxon test. FIG. 6D depicts a northern blot showing Arg-TCT-4-1 overexpression in MEF-WT cells. FIG. 6E is a schematic of Renilla sensor enriched with AGA codons. FIG. 6F examines Renilla reporter activity upon Arg-TCT overexpression. Renilla light units were normalized to firefly luciferase, and empty vector was set to 1. Error bars denote mean±DS. Each experiment corresponds to n=3; each experiment was repeated three times. One-way analysis of variance (ANOVA) with Bonferroni correction. **p<0.01; ns, not significant. FIG. 6G shows the quantification of colony formation in soft agar. Error bars denote mean±SD. Each experiment corresponds to n=3; each experiment was repeated three times. ****p<0.0001; ns, not significant. One-way analysis of variance (ANOVA) and Bonferroni correction. FIG. 6H shows representative pictures of colony formation in soft agar of MEF-WT cell overexpressing Arg-TCT-4-1 wild-type or Arg-TCT-4-1 T34>C mutant. FIG. 6I analyzes a western blot for METTL1 post-overexpression of WT or catalytic-dead METTL1 in primary non-leukemic Nras^{G12D/+} HSPCs. FIG. 6J examines a colony formation assay of primary non-leukemic Nras^{G2D/+} lineage-negative HSPCs, upon ectopic expression of either METTL1 (WT and catalytic-dead_or Arg-TCT-4-1 compared with empty control (mean±SD, n=3). CFU, colony-forming units; ns, not significant; *p<0.01 (t test). FIG. 6K shows bioluminescence imaging of mice transplanted with luciferase-expressing MOLM-13 cells upon overexpression of either METTL1 (WT and catalytic-dead) or Arg-TCT-4-1 compared with empty control at the indicated time point. FIG. 6L quantifies whole-body bioluminescence related to FIG. 6K (mean±SD, n=5). *p<0.01. FIG. 6M depicts a Kaplan-Meier plot showing the survival time of the mice related to FIG. 6K. A log rank test was performed (n=5 animals per group). **p<0.01.

FIGS. 7A-7H. FIG. 7A examines changes in protein abundance between Arg-TCT-4-1 (heavy) overexpressing cells and empty vector (light) control cells measured by SILAC-based proteomics; n=3, moderated t test. FIG. 7B analyzes a Pearson correlation between fold changes in METTL1/WDR4- and Arg-TCT-4-1-overexpressing cells (3,873 proteins that were detected in both groups were included in the analysis. FIG. 7C depicts a Gene Ontology analysis of differentially expressed proteins in METTL/WDR4 and Arg-TCT-4-1-overexpressing cells. FIG. 7D shows a Venn diagram of the overlap of METTL1/WDR4 and Arg-TCT-4-1 proteomic datasets (p<0.05 and FC>1.2). FIG. 7E shows a Gene Ontology analysis of the proteins in the overlap from FIG. 7D. FIG. 7F shows representative western blot analysis of a set of proteins found to be upregulated in METTL1/WDR4 and Arg-TCT-4-1 datasets in MEF-WT cells. FIG. 7G examines relative mRNA expression measured via qRT-PCR in MEF-WT cells. RPLP0 was used as a normalizer, and EV samples were set to 1. N=3 with here technical triplicates; values from unpaired Student's t test (mean±SD). *p<0.05, **p<0.01, and ***p<0.001. FIG. 7H analyzes mCherry/acGFP1 ratios measured by flow cytometry between mCherry-Hmga2-WT (5 of 12 AGA codons) and mCherry-Hmga2-MUT (0 of 12 AGA codons) reporters. Data are mean±SD. N=2. P values from two-way ANOVA with Sidik correction. *p<0.05 and **p<0.01.

FIG. 8 shows the effect of blocking Arg-TCT-4 on colony formation and tumor formation. When Arg-TCT-4 is knocked down, there are less colonies formed as compared to shGFP control and shows the quantification of colony formation in soft agar. Tumor formation is also reduced in shARG samples as compared to shGFP samples.

FIG. 9 depicts Arg-TCT-4 targeting using RNAi. A northern blot shows Arg-TCT-4 expression in human GBM cells (LNZ308). Knockdown using shARG shows accumulation of Arg-TCT-4-1.

FIG. 10 examines accumulation of charged Arg-TCT-4 in a northern blot after treatment with shARG and shGFP control.

FIG. 11 shows an effect of blocking Arg-TCT-4 on protein synthesis using the GBM model. After treatment of shArg, there is less expression of the Hmga2 sensor (enriched with cognate AGA codons) as compared to the Parental and shGFP controls. There is also a positive correlation at the proteome level between the knockdown of METTL1 (using shMETTL1) and the knockdown of Arg-TCT-4-1 (using shArg-TCT-4-1).

FIG. 12 examines how METTL1 overexpression in LPS cells associates with increased abundance of a tRNA Arg-TCT-4. The 93T449 sample has the largest copy number alteration of METTL1 as compared to the other LPS samples. A northern blot demonstrates the increased abundance of LPS in Arg-TCT-4 as compared to Arg-TCT-1,2,3 5.

FIG. 13 depicts how modulation of Arg-TCT-4-1 causes cell growth defects in liposarcoma cells with Arg-TCT-4-1 expression. Plaque assays and quantification shows that knockdown of Arg (using shArg) leads to reduced cell growth as compared to the Parental and shGFP controls in LPS853 but not in LPS141 cells that do not express ARG-TCT-4-1.

FIG. 14 concludes that shArg does not cause changes in aminoacylation in LPS cells.

FIG. 15 shows inhibiting Arg-TCT-4-1 using antisense oligonucleotides causes cell death in a dose-dependent manner.

FIG. 16 concludes that antisense oligonucleotide causes significant increase in apoptosis only in cells with high levels of Arg-TCT-4-1. In two different LPS cell samples (LPS853 and LPS141) and normal human fibroblasts (BJ), Arg-TCT-4-1 is expressed at different levels. Selecting three of these human cell samples, LPS853, LPS141, and BJ, antisense oligonucleotides (ASOs) treatment only in LPS853 cells show an increase in apoptotic cells %.

FIG. 17 shows antisense oligonucleotide causes accumulation of peptidyl-Arg-TCT-4-1.

FIG. 18 analyzes how ASO-1 treated cells show decrease protein synthesis of AGA rich transcript (mCherry-Hmga2).

FIG. 19 shows inhibition of Arg-TCT-4-1 causes translation defects. Inhibition of Arg-TCT-4-1 causes an increase in AGA pauses and a widespread reduction of translation efficiencies in multiple genes.

FIG. 20 shows inhibition of Arg-TCT-4-1 causes translation defects in genes involved in cell division. Gene ontology analyses of genes with differential tranlation efficiencies.

FIG. 21 shows inhibition of Arg-TCT-4-1 causes changes in the proteome. Volcano plots showing changes in protein abundance in a glioblastoma model (left) and a liposarcoma model (right).

FIG. 22 shows inhibition of Arg-TCT-4-1 suppresses tumor formation in vivo in a xenograft model of liposarcoma. Growth curves, tumor size and weight comparison after inhibition of Arg-TCT-4-1.

FIG. 23 shows the inhibition of Arg-TCT-4-1 does not affect AIM2 expression. Arg-TCT-4-1 is harbored within an intronic region of AIM2. Targeting of Arg-TCT-4-1 does not affect AIM2 expression

### DETAILED DESCRIPTION

Provided herein are methods and compositions for treating cancers, particularly cancers characterized by overexpression of an oncogenic tRNA, ARG-TCT-4-1 (e.g., cancers overexpressing methyltransferase-like protein 1 (METTL1)), by inhibiting the activity and/or expression of ARG-TCT-4-1.

### Definitions

As used herein, the term “inhibitory nucleic acid” refers to a nucleic acid molecule which can inhibit the expression of a target, e.g., double-stranded RNAs (dsRNAs), siRNAs, miRNA, antisense oligonucleotides and the like. The use of inhibitory nucleic acids (iNAs) permits the targeted degradation of mRNA transcripts, resulting in decreased expression and/or activity of the target or changes in mRNA processing (e.g., splicing changes). In addition, inhibitory nucleic acids can bind directly to RNA molecules, including transfer RNAs, and either sequester such tRNAs to reduce activity and/or induce breakdown of such tRNAs, effectively reducing both expression and activity of such tRNAs.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female and can be of any age (e.g., fetal, neonate, infant, toddler, child, teenager, adolescent, adult, geriatric etc.).

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a nucleic acid as described herein can be engineered. As used herein, “engineered” refers to the aspect of having been manipulated by the hand of man. For example, a polypeptide is considered to be “engineered” when at least one aspect of the polynucleotide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), reduction in hospitalization visits or length of stay, reduction in the need for medical interventions, improved quality of life and/or decreased mortality, whether detectable or undetectable. The term “treatment” of a disease also includes providing relief from the symptoms or side-effects of the disease (including palliative treatment).

As used herein, the term “pharmaceutical composition” refers to the inhibitor of ARG-TCT-4-1 (e.g., an inhibitory nucleic acid) in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments of any of the aspects, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments of any of the aspects, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and/or affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate anon-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

Inhibitory Nucleic Acids (iNA)

Inhibitory nucleic acids useful in the present methods and compositions include double-stranded RNA, short hairpin RNA (shRNA), small interfering RNA (siRNA), microRNA (miRNA), antisense oligonucleotides, ribozymes, external guide sequence (EGS) oligonucleotides, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid (e.g., an oncogenic tRNA) and modulate its function. In some embodiments of any of the aspects, the inhibitory nucleic acids include antisense RNA, antisense DNA, antisense oligonucleotides, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids), the contents of each of which are incorporated herein by reference in their entirety.

In some embodiments of any of the aspects, an iNA as described herein effects inhibition of the expression and/or activity of a target, e.g., an oncogenic transfer RNA (tRNA). In some embodiments of any of the aspects, contacting a cell with the inhibitor (e.g. an iNA) results in a decrease in the target tRNA activity and/or tRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iNA. In some embodiments of any of the aspects, administering an inhibitor (e.g. an iNA) to a subject results in a decrease in the target tRNA activity and/or expression level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iNA.

In some embodiments of any of the aspects, the iNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. In other embodiments, the target sequence is derived from the sequence of a target transfer RNA, particularly an oncogenic tRNA, such as ARG-TCT-4-1. In one embodiment, the target sequence of ARG-TCT-4-1 comprises SEQ ID NO: 1 or SEQ ID NO: 2. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 22 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage or sequestration will most often be part of a larger RNA molecule, such as a tRNA or mRNA molecule. Where relevant, a “part” of an mRNA or tRNA target is a contiguous sequence of an mRNA or tRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target RNA sequence will be at least 15 nucleotides in length, preferably 15-30 nucleotides in length.

In some embodiments of any of the aspects, the inhibitory nucleic acid domain specifically binds to an oncogenic tRNA, such as ARG-TCT-4-1. One of ordinary skill in the art is aware of how to design and produce inhibitory nucleic acids that inhibit e.g., ARG-TCT-4-1. Exemplary, non-limiting examples of inhibitory nucleic acid domain sequences are provided below herein.

In certain embodiments, the inhibitory nucleic acid described herein comprise a target sequence having a region of complementarity to ARG-TCT-4-1 as shown in Table 1. In other embodiments, the inhibitory nucleic acid comprises, consists of, or consists essentially a target sequence having at least 80%, at least 85%, at least 90%, at least 95%, at least 98% or greater sequence identity to a sequence selected from SEQ ID NO: 1 or 2.

TABLE 1
Exemplary targeting sequences

| SEQ | | |
|---|---|---|
| ID NO | Target | Exemplary targeting sequence |
| 1 | ARG-TCT-4-1 | GAGCGCGCTGGACTTCT |
| 2 | ARG-TCT-4-1 | GACTTCTAATCCAGAGG |

In other embodiments, an inhibitory nucleic acid (e.g., an shRNA) as described herein comprises, consists of or consists essentially of a sequence having at least 80%, at least 90%, at least 95%, at least 98% or greater sequence identity to a sequence selected from SEQ ID NO: 3 or 4.

TABLE 2
Exemplary shRNA sequences

| SEQ | | |
|---|---|---|
| ID NO | Target | Exemplary shRNA sequence |
| 3 | ARG-TCT- | CCGGAAGAGCGCGCTGGACTTCTAACTCGAGTT |
| | 4-1 | AGAAGTCCAGCGCGCTCTTTTTTTG |
| 4 | ARG-TCT- | CCGGAAGACTTCTAATCCAGAGGTTCTCGAGAA |
| | 4-1 | CCTCTGGATTAGAAGTCTTTTTTTG |

In other embodiments, the inhibitory nucleic acid targeting ARG-TCT-4-1 comprises an antisense oligonucleotide that comprises, consists of, or consists essentially of a sequence having at least 80%, at least 90%, at least 95%, at least 98% or greater sequence identity to a sequence selected from SEQ ID NO: 5, 6 or 7.

TABLE 3
Exemplary antisense oligonucleotide sequences

| SEQ | | Exemplary antisense |
|---|---|---|
| ID NO | Target | oligonucleotide sequence |
| 5 | ARG-TCT-4-1 | +A + G + CGCGCTCGTCC + A + |
| | | T + T |
| 6 | ARG-TCT-4-1 | +T + C + TCTGCCGGGAC + T + |
| | | C + G |
| 7 | ARG-TCT-4-1 | +A + A + GTCCAGCGCGC + T + |
| | | C + G |

“+” =locked nucleic acid

As one of skill in the art will recognize, not all transfer RNAs are oncogenic and it is not advisable to inhibit tRNA activity of non-oncogenic tRNAs. Thus, when designing an inhibitory nucleic acid against e.g., ARG-TCT-4-1, one should take care to reduce or eliminate binding to other tRNAs. This can be achieved by careful selection of the target sequence of the inhibitory nucleic acid such that the binding to ARG-TCT-4-1 is specific (e.g., little to no binding to other tRNAs) or selective (e.g., binding to ARG-TCT-4-1 occurs at concentrations that are at least 2-fold lower (e.g., at least 5-fold, at least 10 fold, at least 20-fold, at least 100-fold or more) than concentrations that bind to or inhibit other tRNAs).

A double-stranded inhibitory nucleic acid as described herein can further include one or more single-stranded nucleotide overhangs. The double-stranded inhibitory nucleic acid can be synthesized by standard methods known in the art as further discussed below, e.g., by use of an automated DNA synthesizer, such as are commercially available from, for example, Biosearch, Applied Biosystems, Inc. In some embodiments of any of the aspects, the antisense strand of a double-stranded inhibitory nucleic acid has a 1-10 nucleotide overhang at the 3′ end and/or the 5′ end. In some embodiments of any of the aspects, the sense strand of a double-stranded inhibitory nucleic acid has a 1-10 nucleotide overhang at the 3′ end and/or the 5′ end. In some embodiments of any of the aspects, at least one end of a double-stranded inhibitory nucleic acid has a single-stranded nucleotide overhang of 1 to 4, generally 1 or 2 nucleotides. Double-stranded inhibitory nucleic acids having at least one nucleotide overhang have unexpectedly superior inhibitory properties relative to their blunt-ended counterparts. In some embodiments of any of the aspects, one or more of the nucleotides in the overhang is replaced with a nucleoside thiophosphate.

As used herein, the term “nucleotide overhang” refers to at least one unpaired nucleotide that protrudes from the duplex structure of an inhibitory nucleic acid, e.g., a dsRNA. For example, when a 3′-end of one strand of a double-stranded inhibitory nucleic acid extends beyond the 5′-end of the other strand, or vice versa, there is a nucleotide overhang. A double-stranded inhibitory nucleic acid can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a double-stranded inhibitory nucleic acid.

The terms “blunt” or “blunt ended” as used herein in reference to a double-stranded inhibitory nucleic acid mean that there are no unpaired nucleotides or nucleotide analogs at a given terminal end of a dsRNA, i.e., no nucleotide overhang. One or both ends of a double-stranded inhibitory nucleic acid can be blunt. Where both ends of a double-stranded inhibitory nucleic acid are blunt, the double-stranded inhibitory nucleic acid is said to be blunt ended. To be clear, a “blunt ended” double-stranded inhibitory nucleic acid is a double-stranded inhibitory nucleic acid that is blunt at both ends, i.e., no nucleotide overhang at either end of the molecule. Most often such a molecule will be double-stranded over its entire length.

In this aspect, one of the two strands is complementary to the other of the two strands, with one of the strands being substantially complementary to a sequence of the target tRNA, mRNA or miRNA. As such, in this aspect, a double-stranded inhibitory nucleic acid will include two oligonucleotides, where one oligonucleotide is described as the sense strand and the second oligonucleotide is described as the corresponding antisense strand of the sense strand. As described elsewhere herein and as known in the art, the complementary sequences of a double-stranded inhibitory nucleic acid can also be contained as self-complementary regions of a single nucleic acid molecule, as opposed to being on separate oligonucleotides. In some embodiments, only a portion the molecule, e.g., the inhibitory nucleic acid domain is a double-stranded molecule.

The skilled person is well aware that inhibitory nucleic acid having a duplex structure of between 20 and 23, but specifically 21, base pairs have been hailed as particularly effective in inducing antisense-mediated inhibition (Elbashir et al., EMBO 2001, 20:6877-6888). However, others have found that shorter or longer inhibitory nucleic acids can be effective as well.

Further, it is contemplated that for any sequence identified, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of inhibitory nucleic acids based on those target sequences in an inhibition assay as known in the art can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An inhibitory nucleic acid as described herein can contain one or more mismatches to the target sequence. In some embodiments of any of the aspects, an inhibitory nucleic acid as described herein contains no more than 3 mismatches. If the antisense strand of the inhibitory nucleic acid contains mismatches to a target sequence, it is preferable that the area of mismatch not be located in the center of the region of complementarity. If the antisense strand of the inhibitory nucleic acid contains mismatches to the target sequence, it is preferable that the mismatch be restricted to be within the last 5 nucleotides from either the 5′ or 3′ end of the region of complementarity. For example, for a 23 nucleotide inhibitory nucleic acid agent strand which is complementary to a region of the target gene or a precursor thereof, the strand generally does not contain any mismatch within the central 13 nucleotides. The methods described herein or methods known in the art can be used to determine whether an inhibitory nucleic acid containing a mismatch to a target sequence is effective in inhibiting the expression of the target gene. Consideration of the efficacy of inhibitory nucleic acids with mismatches in inhibiting expression of the target gene is important, especially if the particular region of complementarity in the target gene is known to have polymorphic sequence variation within the population.

### Nucleic Acid Modifications

In some embodiments of any of the aspects described herein, the nucleic acid of an iNA, e.g., an shRNA, an siRNA, an miRNA, a dsRNA or an antisense oligonucleotide, is chemically modified to enhance stability or other beneficial characteristics. In some embodiments, the inhibitory nucleic acid is partially or fully modified. The nucleic acids described herein can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or non-natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-[wherein the native phosphodiester backbone is represented as —O—P—O—CH2-].

In other embodiments, RNA mimetics suitable or contemplated for use in iNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

The RNA of an iRNA can also be modified to include one more unlocked nucleic acids (UNA). UNAs are acyclic derivatives of RNA lacking the C2′-C3′ bond of the ribose ring. See, e.g., Langkjaer et al. Bioorganic & Medicinal Chemistry 2009 17:5420-5. An UNA at the 5′ end of a RNA molecule can improve iRNA targeting, see e.g., Snead et al. Molecular Therapy Nucleic Acids 2013 2:E103. In some embodiments, the 5′ position of the inhibitory nucleic acid is a UNA.

Modified RNAs can also contain one or more substituted sugar moieties. The iRNAs, e.g., dsRNAs, described herein can include one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)·nOCH3, O(CH2)nNH2, O(CH2)nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-23 Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, a ligand alters the distribution, targeting or lifetime of an inhibitory nucleic acid agent into which it is incorporated. In some embodiments of any of the aspects, a ligand provides an enhanced affinity for a selected target, e.g, molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide. In one embodiment, the ligand comprises folate or GalNac.

Ligands can also include targeting groups, e.g., a cell or tissue targeting agent, e.g., a lectin, glycoprotein, lipid or protein, e.g., an antibody, that binds to a specified cell type such as a hepatocyte or a macrophage, among others. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP

Ligands can be proteins, e.g., glycoproteins, or peptides, e.g., molecules having a specific affinity for a co-ligand, or antibodies e.g., an antibody, that binds to a specified cell type such as a hepatocyte or macrophage. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose.

The ligand can be a substance, e.g, a drug, which can increase the uptake of the inhibitory nucleic acid agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments of any of the aspects, a ligand attached to an inhibitory nucleic acid as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

For macromolecular drugs and hydrophilic drug molecules, which cannot easily cross bilayer membranes, entrapment in endosomal/lysosomal compartments of the cell is thought to be the biggest hurdle for effective delivery to their site of action. A number of approaches and strategies have been devised to address this problem. For liposomal formulations, the use of fusogenic lipids in the formulation have been the most common approach (Singh, R. S., Goncalves, C. et al. (2004). On the Gene Delivery Efficacies of pH-Sensitive Cationic Lipids via Endosomal Protonation. A Chemical Biology Investigation. Chem. Biol. 11, 713-723.). Other components, which exhibit pH-sensitive endosomolytic activity through protonation and/or pH-induced conformational changes, include charged polymers and peptides. Examples may be found in Hoffman, A. S., Stayton, P. S. et al. (2002). Design of “smart” polymers that can direct intracellular drug delivery. Polymers Adv. Technol. 13, 992-999; Kakudo, Chaki, T., S. et al. (2004). Transferrin-Modified Liposomes Equipped with apH-Sensitive Fusogenic Peptide: An Artificial Viral-like Delivery System. Biochemistry 436, 5618-5628; Yessine, M. A. and Leroux, J. C. (2004). Membrane-destabilizing polyanions: interaction with lipid bilayers and endosomal escape of biomacromolecules. Adv. Drug Deliv. Rev. 56, 999-1021; Oliveira, S., van Rooy, I. et al. (2007). Fusogenic peptides enhance endosomal escape improving inhibitory nucleic acid-induced silencing of oncogenes. Int. J. Pharm. 331, 211-4. They have generally been used in the context of drug delivery systems, such as liposomes or lipoplexes. For folate receptor-mediated delivery using liposomal formulations, for instance, a pH-sensitive fusogenic peptide has been incorporated into the liposomes and shown to enhance the activity through improving the unloading of drug during the uptake process (Turk, M. J., Reddy, J. A. et al. (2002). Characterization of a novel pH-sensitive peptide that enhances drug release from folate-targeted liposomes at endosomal pHs is described in Biochim. Biophys. Acta 1559, 56-68).

The inhibitory nucleic acids described herein can be conjugated or bound to macromolecules to extend their half-life. Suitable macromolecules include cholesterol, PEG, a liposome, or Fc.

In certain embodiments, the endosomolytic components can be polyanionic peptides or peptidomimetics which show pH-dependent membrane activity and/or fusogenicity. A peptidomimetic can be a small protein-like chain designed to mimic a peptide. A peptidomimetic can arise from modification of an existing peptide in order to alter the molecule's properties, or the synthesis of a peptide-like molecule using unnatural amino acids or their analogs. In certain embodiments, they have improved stability and/or biological activity when compared to a peptide. In certain embodiments, the endosomolytic component assumes its active conformation at endosomal pH (e.g., pH 5-6). The “active” conformation is that conformation in which the endosomolytic component promotes lysis of the endosome and/or transport of the inhibitory nucleic acid from the endosome to the cytoplasm of the cell.

Exemplary endosomolytic components include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic component can contain a chemical group (e.g., an amino acid) which will undergo a change in charge or protonation in response to a change in pH. The endosomolytic component may be linear or branched. Exemplary primary sequences of endosomolytic components include H2N-(AALEALAEALEALAEALEALAEAAAAGGC)—CO2H (SEQ ID NO: 8); H2N-(AALAEALAEALAEALAEALAEALAAAAGGC)—CO2H (SEQ ID NO: 9); and H2N-(ALEALAEALEALAEA)-CONH2 (SEQ ID NO: 10).

In certain embodiments, more than one endosomolytic component can be incorporated into the inhibitory nucleic acid agent of the invention. In some embodiments of any of the aspects, this will entail incorporating more than one of the same endosomolytic component into the inhibitory nucleic acid agent. In other embodiments, this will entail incorporating two or more different endosomolytic components into inhibitory nucleic acid agent.

These endosomolytic components can mediate endosomal escape by, for example, changing conformation at endosomal pH. In certain embodiments, the endosomolytic components can exist in a random coil conformation at neutral pH and rearrange to an amphipathic helix at endosomal pH. As a consequence of this conformational transition, these peptides can insert into the lipid membrane of the endosome, causing leakage of the endosomal contents into the cytoplasm. Because the conformational transition is pH-dependent, the endosomolytic components can display little or no fusogenic activity while circulating in the blood (pH ˜7.4). “Fusogenic activity,” as used herein, is defined as that activity which results in disruption of a lipid membrane by the endosomolytic component. One example of fusogenic activity is the disruption of the endosomal membrane by the endosomolytic component, leading to endosomal lysis or leakage and transport of one or more components of the modular composition of the invention (e.g., the nucleic acid) from the endosome into the cytoplasm.

Suitable endosomolytic components can be tested and identified by a skilled artisan. For example, the ability of a compound to respond to, e.g., change charge depending on, the pH environment can be tested by routine methods, e.g., in a cellular assay. In certain embodiments, a test compound is combined with or contacted with a cell, and the cell is allowed to internalize the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in the endosome population in the cells. The test compound and/or the endosomes can be labeled, e.g., to quantify endosomal leakage.

In another type of assay, an inhibitory nucleic acid agent described herein is constructed using one or more test or putative fusogenic agents. The inhibitory nucleic acid agent can be labeled for easy visualization. The ability of the endosomolytic component to promote endosomal escape, once the inhibitory nucleic acid agent is taken up by the cell, can be evaluated, e.g., by preparation of an endosome preparation, or by microscopy techniques, which enable visualization of the labeled inhibitory nucleic acid agent in the cytoplasm of the cell. In certain other embodiments, the inhibition of gene expression, or any other physiological parameter, may be used as a surrogate marker for endosomal escape.

In some embodiments of the aspects described herein, a ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

In another aspect, the ligand is a cell-permeation agent, preferably a helical cell-permeation agent. Preferably, such agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

Peptides suitable for modification of inhibitory nucleic acids can comprise a natural peptide, e.g., tat or antennopedia peptide, a synthetic peptide, or a peptidomimetic. Furthermore, the peptide can be a modified peptide, for example peptide can comprise non-peptide or pseudo-peptide linkages, and D-amino acids. A peptidomimetic (also referred to herein as an oligopeptidomimetic) is a molecule capable of folding into a defined three-dimensional structure similar to a natural peptide. The attachment of peptide and peptidomimetics to inhibitory nucleic acid agents can affect pharmacokinetic distribution of the inhibitory nucleic acid, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIWFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In some embodiments of any of the aspects, the inhibitory nucleic acid oligonucleotides described herein further comprise carbohydrate conjugates. The carbohydrate conjugates are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8). In some embodiments of any of the aspects, the carbohydrate conjugate further comprises other ligand such as, but not limited to, PK modulator, endosomolytic ligand, and cell permeation peptide.

In some embodiments, the inhibitory nucleic acid is fused to another moiety as described herein, optionally through the use of a linker. The term “linker” or “linking group” means a moiety (e.g., an organic moiety) that connects two parts of a compound. In some embodiments of any of the aspects, a linker can be a polypeptide or a nucleic acid that functions to attach two domains or moieties. A linker can comprise, for example, 1 to 1000 nucleotides or more. In some embodiments of any of the aspects, the linker comprises 1-100, 10-100, 100-900, 200-800, 300-700, 500-1000, or 700-1000 nucleotides. In some embodiments of any of the aspects, a linker can be 1-10 nucleotides in length, e.g., 1-5 nucleotides or 3 nucleotides in length. The length of the linker can be optimized for one or more desired properties (e.g., separation of the domains, prevention of self-hybridization, etc.).

In some embodiments of any of the aspects, linkers can comprise a direct bond or an atom such as carbon, oxygen, or sulfur, a unit such as NR8, C(O), C(O)NH, SO, SO2, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO2, N(R8), C(O), substituted or unsubstituted aryl, substituted or unsubstituted heteroaryl, substituted or unsubstituted heterocyclic; where R8 is hydrogen, acyl, aliphatic or substituted aliphatic. In some embodiments of any of the aspects, the linker is between 1-24 atoms, preferably 4-24 atoms, preferably 6-18 atoms, more preferably 8-18 atoms, and most preferably 8-16 atoms.

A cleavable linking group is one which is sufficiently stable outside the cell, but which upon entry into a target cell is cleaved to release the two parts the linker is holding together. In some embodiments of any of the aspects, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. Further examples of cleavable linking groups include but are not limited to, redox-cleavable linking groups (e.g. a disulphide linking group (—S—S—)), phosphate-based cleavable linkage groups, ester-based cleavable linking groups, and peptide-based cleavable linking groups. Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments of any of the aspects, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

It is not necessary for all positions in a given compound to be uniformly modified, and in fact more than one of the aforementioned modifications can be incorporated in a single compound or even at a single nucleoside within an inhibitory nucleic acid.

In some embodiments of any of the aspects, the inhibitory nucleic acids described herein can comprise at least one region wherein the nucleic acid is modified so as to confer upon the inhibitory nucleic acid increased resistance to nuclease degradation, increased cellular uptake, and/or increased binding affinity for the target nucleic acid. An additional region of the inhibitory nucleic acid can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibitory nucleic acid inhibition of gene expression. In some embodiments, fully modified molecules that do not posses RNAse H activity may be desirable to clock the tRNA. Consequently, comparable results can often be obtained with shorter inhibitory nucleic acids when chimeric inhibitory nucleic acids are used, compared to, e.g., phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the nucleic acid of an inhibitory nucleic acid can be modified by a non-ligand group. A number of non-ligand molecules have been conjugated to inhibitory nucleic acids in order to enhance the activity, cellular distribution or cellular uptake of the inhibitory nucleic acid, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or apolyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such nucleic acid conjugates have been listed above. Typical conjugation protocols involve the synthesis of a nucleic acid bearing an aminolinker at one or more positions of the sequence. The amino group is then reacted with the molecule being conjugated using appropriate coupling or activating reagents. The conjugation reaction may be performed either with the nucleic acid still bound to the solid support or following cleavage of the nucleic acid, in solution phase. Purification of the nucleic acid conjugate by HPLC typically affords the pure conjugate.

### Lipid Compositions and Nanoparticles

Exemplary lipid compositions and lipid nanoparticle for delivering inhibitory nucleic acids to cells or subjects are known to those of skill in the art and are not described in detail herein. Briefly, methods for the introduction of vectors or constructs into cells include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

In some embodiments, the inhibitory nucleic acids described herein can be administered using a micelle or liposome. Amphiphilic polymers form a micelle structure in an aqueous solution since the water solubility of their hydrophilic moiety greatly differs from that of their hydrophobic moiety. In the aqueous solution, the micelle has a unique core-shell structure wherein the hydrophobic moieties form an inner core and the hydrophilic moieties form an outer shell. The inner cores of such micelles can be filled with an inhibitory nucleic acid, thereafter which would show a greatly-enhanced water solubility and an extended duration of a therapeutic effect. Furthermore, it is possible to control drug distribution in a body depending on the size of the micelle and to deliver a drug onto a target depending on the surface properties thereof.

The term “liposome” refers to a synthetic entity or vesicle, formed of at least one lipid bilayer membrane (or matrix) enclosing an aqueous compartment. Liposomes can be unilamellar (a single bilayer membrane) or multilamellar (several membranes layered like an onion). The lipids constituting the bilayer membrane comprise a nonpolar region which, typically, is made of chain(s) of fatty acids or of cholesterol and a polar region (e.g., lipid A molecules described herein and the like), typically made of a phosphate group and/or of tertiary or quaternary ammonium salts. Depending on its composition, the polar region may, in particular at physiological pH (pH≈7) carry either a negative (anionic lipid) or positive (cationic lipid) net (overall) surface charge, or not carry a net charge (neutral lipid).

Any type of liposome can be used to encapsulate or permit inhibitory nucleic acid binding to the shell for delivery and can be constituted of any lipid known to be of use in the production of liposomes. The lipid(s) that go(es) to make up the composition of the liposomes can be neutral, anionic or cationic lipid(s); the latter being preferred. These lipids can be of natural origin (plant or egg extraction products, for example) or synthetic origin. The liposomes can also be constituted of a mixture of these lipids; for example, of a cationic or anionic lipid or a neutral lipid, as a mixture. When a mixture of lipids is used in a drug delivery particle, the neutral lipid is often referred to as a colipid. In one embodiment, the charged (cationic or anionic) lipid: neutral lipid molar ratio is between 10:1 and 1:10, advantageously between 4:1 and 1:4, preferably between 3:1 and 1:3, limits included.

Non-limiting examples of neutral lipids include: (i) cholesterol; (ii) phosphatidylcholines such as, for example, 1,2-diacyl-sn-glycero-3-phosphocholines, e.g. 1,2-dioleoyl-sn-glycero-3-phosphocholine (DOPC), and also 1-acyl-2-acyl-sn-glycero-3-phosphocholines of which the acyl chains are different than one another (mixed acyl chains); and (iii) phosphatidylethanolamines such as, for example, 1,2-diacyl-sn-glycero-3-phosphoethanolamines, e.g. 1,2-dioleoyl-sn-glycero-3-phosphoethanolamine (DOPE), and also 1-acyl-2-acyl-sn-glycero-3-phosphoethanolamines bearing mixed acyl chains.

Exemplary anionic lipids include, but are not limited to: (i) cholesteryl hemisuccinate (CHEMS); (ii) phosphatidylserines such as 1,2-diacyl-sn-glycero-3-[phospho-L-serine]s, e.g. 1,2-dioleoyl-sn-glycero-3-[phospho-L-serine](DOPS), and 1-acyl-2-acyl-sn-glycero-3-[phospho-L-serine]s bearing mixed acyl chains; (iii) phosphatidylglycerols such as 1,2-diacyl-sn-glycero-3-[phospho-rac-(1-glycerol)]s, e.g. 1,2-dioleoyl-sn-glycero-3-[phospho-rac-(1-glycerol)](DOPG), and 1-acyl-2-acyl-sn-glycero-3-[phospho-rac-(1-glycerol)]s bearing mixed acyl chains; (iv) phosphatidic acids such as 1,2-diacyl-sn-glycero-3-phosphates, e.g. 1,2-dioleoyl-sn-glycero-3-phosphate (DOPA), and 1-acyl-2-acyl-sn-glycero-3-phosphates bearing mixed acyl chains; and (v) phosphatidylinositols such as 1,2-diacyl-sn-glycero-3-(phosphoinositol)s, e.g. 1,2-dioleoyl-sn-glycero-3-(phosphoinositol) (DOPI), and 1-acyl-2-acyl-sn-glycero-3-(phosphoinositol)s bearing mixed acyl chains.

Non-limiting examples of cationic lipids include but are not limited to: (i) lipophilic amines or alkylamines such as, for example, dimethyldioctadecylammonium (DDA), trimethyldioctadecylammonium (DTA) or structural homologs of DDA and of DTA [these alkylamines are advantageously used in the form of a salt; for example, of dimethyldioctadecylammonium bromide (DDAB)]; (ii) octadecenoyloxy(ethyl-2-heptadecenyl-3-hydroxyethyl)imidazolinium (DOTIM) and structural homologs thereof; (iii) lipospermines such as N-palmitoyl-D-erythrosphingosyl-1-O-carbamoylspermine (CCS) and dioctadecylamidoglycylspermine (DOGS, transfectam); (iv) lipids incorporating an ethylphosphocholine structure, such as cationic derivatives of phospholipids, in particular phosphoric ester derivatives of phosphatidylcholine, for example those described in patent application WO 05/049080 and including, in particular:
1,2-dimyristoyl-sn-glycero-3-ethylphosphocholine,
1,2-dipalmitoyl-sn-glycero-3-ethylphosphocholine,
1,2-palmitoyloleoyl-sn-glycero-3-ethylphosphocholine,
1,2-distearoyl-sn-glycero-3-ethylphosphocholine (DSPC),
1,2-dioleyl-sn-glycero-3-ethylphosphocholine (DOEPC or EDOPC or ethyl-DOPC or ethyl PC),
and also structural homologs thereof;
   (v) lipids incorporating a trimethylammonium structure, such as N-(1-[2,3-dioleyloxy]propyl)-N,N,N-trimethylammonium (DOTMA) and structural homologs thereof and those incorporating a trimethylammonium propane structure, such as 1,2-dioleyl-3-trimethylammonium propane (DOTAP) and structural homologs thereof; and also lipids incorporating a dimethylammonium structure, such as 1,2-dioleyl-3-dimethylammonium propane (DODAP) and structural homologs thereof; and (vi) cationic derivatives of cholesterol, such as 3β-[N—(N′,N′-dimethylaminoethane)-carbamoyl]cholesterol (DC-Chol) or other cationic derivatives of cholesterol, such as those described in U.S. Pat. No. 5,283,185, and in particular cholesteryl-3β-carboxamidoethylenetrimethylammonium iodide, cholesteryl-3β-carboxyamidoethylene-amine, cholesteryl-3β-oxysuccinamidoethylenetrimethylammonium iodide and 3β-[N-(polyethyleneimine)carbamoyl]cholesterol.

The term “structural homologs” signifies lipids which have the characteristic structure of the reference lipid while at the same time differing therefrom by virtue of secondary modifications, especially in the nonpolar region, in particular of the number of carbon atoms and of double bonds in the fatty acid chains. These fatty acids, which are also found in the neutral and anionic phospholipids, are, for example, dodecanoic or lauric acid (C12:0), tetradecanoic or myristic acid (C14:0), hexadecanoic or palmitic acid (C16:0), cis-9-hexadecanoic or palmitoleic acid (C16:1), octadecanoic or stearic acid (C18:0), cis-9-octadecanoic or oleic acid (C18:1), cis,cis-9,12-octadecadienoic or linoleic acid (C18:2), cis-cis-6,9-octadecadienoic acid (C18:2), all-cis-9,12,15-octadecatrienoic or cA-linolenic acid (C18:3), all-cis-6,9,12-octadecatrienoic or γ-linolenic acid (C18:3), eicosanoic or arachidic acid (C20:0), cis-9-eicosenoic or gadoleic acid (C20:1), all-cis-8,11,14-eicosatrienoic acid (C20:3), all-cis-5,8,11,14-eicosatetraenoic or arachidonic acid (C20:4), all-cis-5,8,11,14,17-eicosapentaneoic acid (C20:5), docosanoic or behenic acid (C22:0), all-cis-7,10,13,16,19-docosapentaenoic acid (C22:5), all-cis-4,7,10,13,16,19-docosahexaenoic acid (C22:6) and tetracosanoic or lignoceric acid (C24:0).

Microparticles and Nanoparticles: In some embodiments, the inhibitory nucleic acids as described herein are incorporated into or on a “targeting particle,” which are substantially spherical bodies or membranous bodies from 500 mn-999 m in size, such as e.g., liposomes, micelles, exosomes, microbubbles, or unilamellar vesicles. In some embodiments, the particle is less than 900 μm, less than 800 μm, less than 700 μm, less than 600 μm, less than 500 μm, less than 400 μm, less than 300 μm, less than 200 μm, less than 100 μm, less than 90 μm, less than 80 μm, less than 75 μm, less than 70 μm, less than 60 μm, less than 50 μm, less than 40 μm, less than 30 μm, less than 25 μm, less than 20 μm, less than 15 μm, less than 10 μm, less than 5 μm, less than 2 μm, less than 1 μm, less than 750 nm, less than 500 nm or smaller. As will be readily understood by those of skill in the art, a targeting particle that is of nanometer size (e.g., 10 to 1000 nm) is also referred to herein as a “nanoparticle.”

Nanoparticles can be solid, colloidal particles consisting of macromolecular substances that vary in size from 10-1000 nanometers. An inhibitory nucleic acid can be entrapped, suspended, adsorbed, attached or encapsulated into the nanoparticle matrix for delivery (including targeted delivery) for therapeutic treatment of a given disease (e.g., cancer).

Targeted delivery of nanoparticles can be achieved by either passive or active targeting. Active targeting of an inhibitory nucleic acid is achieved by including a moiety that recognizes and binds to a tissue or cell-specific ligand (Lamprecht et al., Biodegradable nanoparticles for targeted drug delivery in treatment of inflammatory bowel disease, J Pharmacol Exp Ther. 299:775-81, 2002). Passive targeting is achieved by coupling the therapeutic agent to a macromolecule that passively reaches the target organ or cell type (Monsky W L et al., Augmentation of transvascular transport of macromolecules and nanoparticles in tumors using vascular endothelial growth factor, Cancer Res. 59:4129-35, 1999).

Exemplary nanoparticles for use in delivering the inhibitory nucleic acids described herein can be prepared preferably using biodegradable materials, however any suitable material can be used in the preparation of drug-delivery nanoparticles including, but not limited to, polymers, lipids (e.g., hydrogenated soy phosphatidylcholine (HSPC), egg phosphatidylcholine (EPC), phosphatidyl ethanolamine (PE), phosphatidyl glycerol (PG), phosphatidyl inositol (PI), monosialogangolioside, sphingomyelin (SPM), distearoylphosphatidylcholine (DSPC), dimyristoylphosphatidylcholine (DMPC), or dimyristoylphosphatidylglycerol (DMPG)), metals (e.g., gold, silver, or a magnetic nanoparticle), etc. Representative, non-limiting examples of biodegradable polymers include synthetic polymers such as polymers of lactic acid and glycolic acid, polyanhydrides, poly(ortho)esters, polyurethanes, poly(hydroxybutiric acid), poly(valeric acid), and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin, and other hydrophilic proteins. The compositions described herein can also comprise bioerodible hydrogels which are prepared from materials and combinations of materials such as polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Preferred biodegradable polymers are polyglycolic acid, polylactic acid, copolymers of glycolic acid and L- or D,L-lactic acid, and copolymers of glycolide and L- or D,L-lactide.

The compositions described herein for delivering an inhibitory nucleic acid can also include a conjugate of a lipid and a hydrophilic polymer, referred to as a ‘lipopolymer.’ Lipopolymers can be obtained commercially or can be synthesized using known procedures. For example, lipopolymers comprised of methoxy(polyethylene glycol) (mPEG) and a phosphatidylethanolamine (e.g., dimyristoyl phosphatidylethanolamine, dipalmitoyl phosphatidylethanolamine, 1,2-distearoyl-3-sn-glycerophosphoethanolamine (distearoyl phosphatidylethanolamine (DSPE)), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates are known in the art and are not described in detail herein.

The hydrophobic component of the lipopolymer can be virtually any hydrophobic compound having or modified to have a chemical group suitable for covalent attachment of a hydrophilic polymer chain. Exemplary chemical groups are, for example, an amine group, a hydroxyl group, an aldehyde group, and a carboxylic acid group. Preferred hydrophobic components are lipids, such as cholesterol, cholesterol derivatives, sphingomyelin, and phospholipids, such as phosphatidylcholine (PC), phosphatidylethanolamine (PE), phosphatidylglycerol (PG), phosphatidic acid (PA), phosphatidylinositol (PI), where the two hydrocarbon chains are typically between about 8-24 carbon atoms in length, and have varying degrees of unsaturation. These lipids are exemplary and are not intended to be limiting, as those of skill can readily identify other lipids that can be covalently modified with a hydrophilic polymer and incorporated into the particles described herein. In some embodiments, the lipopolymer is formed of polyethylene-glycol and a lipid, such as distearoyl phosphatidylethanolamine (DSPE), PEG-DSPE. PEG-DSPE has some degree of biodegradability in vivo, by virtue of the hydrolysable bonds between the fatty acids and the glycerol moiety.

### Targeting of Inhibitors Using Antibodies or Antigen-Binding Fragments Thereof

Although targeted delivery can enhance efficiency of delivery to a given site, it is important to note that it is not necessary because the oncogenic tRNA, ARG-TCT-4-1, is present only in cancer cells and is not expressed in normal cells. By way of this specific pattern of expression, the incidence of off-target effects or side effects is expected to be negligible. As such, the inhibitory nucleic acids described herein are expected to have an acceptable safety profile.

In some embodiments, the inhibitory nucleic acids described herein can be targeted to a cancer cell upon administration to a subject by way of a targeting moiety. As used herein, the term “targeting moiety” refers to a functional group which acts to target or direct an inhibitory nucleic acid or a nanoparticle (e.g., lipid nanoparticle) to a particular location, cell type, diseased tissue, or association, and permits concentration or accumulation at a given site. In general, the “targeting moiety” is directed against a target molecule and allows concentration of the inhibitory nucleic acids in a particular site within a subject. In certain embodiments, the targeting moiety can comprise a binding pair, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; and other targeting moieties include for example, aptamers, receptors, ligands, and fusion proteins.

In certain embodiments, the targeting moiety can be attached directly to the inhibitory nucleic acid or through the use of a linker. In other embodiments, the targeting moiety is attached (e.g., either directly or via a linker) to a nanoparticle comprising the inhibitory nucleic acids. In some embodiments, the targeting moiety comprises an antibody or antigen binding fragment thereof, or an antibody reagent (e.g., a nanobody, an scFv etc.) that recognizes a cancer cell marker.

The following definitions serve to define terms related to antibody or antibody-related targeting moieties.

As used herein, the term “antibody reagent” refers to a polypeptide that includes at least one immunoglobulin variable domain or immunoglobulin variable domain sequence and which specifically binds a given antigen (e.g., a cancer cell marker). An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety.

### Compositions, Formulations and Packaging

Also provided herein are compositions, including pharmaceutical compositions, comprising an inhibitory nucleic acid as described herein. In one embodiment, the compositions are pharmaceutical compositions. Pharmaceutical compositions for use with the methods described herein can be formulated in conventional manner using one or more physiologically acceptable carriers or excipients. Thus, the compounds and their physiologically acceptable salts and solvates can be formulated for administration by, for example, by aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

In some embodiments of any of the aspects, the technology described herein relates to a pharmaceutical composition comprising at least one inhibitory nucleic acid that binds and reduces activity of ARG-TCT-4-1 as described herein, and optionally a pharmaceutically acceptable carrier. In some embodiments of any of the aspects, the active ingredients of the pharmaceutical composition comprise at least one inhibitory nucleic acid that binds and reduces activity of ARG-TCT-4-1 as described herein. In some embodiments of any of the aspects, the active ingredients of the pharmaceutical composition consist essentially of at least one inhibitory nucleic acid that binds and reduces activity of ARG-TCT-4-1 as described herein. In some embodiments of any of the aspects, the active ingredients of the pharmaceutical composition consist of at least one inhibitory nucleic acid that binds and reduces activity of ARG-TCT-4-1 as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C2 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent, e.g. at least one inhibitory nucleic acid that binds and reduces activity of ARG-TCT-4-1 as described herein.

In some embodiments of any of the aspects, the pharmaceutical composition comprising at least one inhibitor of ARG-TCT-4-1 as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of at least one inhibitory nucleic acid that binds to and inhibits ARG-TCT-4-1 are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of the active ingredient as disclosed herein can also be incorporated into the parenteral dosage forms of the disclosure, including conventional and controlled-release parenteral dosage forms.

Pharmaceutical compositions comprising at least inhibitory nucleic acid that binds to and inhibits ARG-TCT-4-1 can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the at least one inhibitory nucleic acid that binds to and inhibits ARG-TCT-4-1 can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In embodiments where the inhibitor of ARG-TCT-4-1 comprises an inhibitory nucleic acid, the nucleic acid can be mixed with a delivery system, such as a liposome system, and optionally can include an acceptable excipient. In a preferred embodiment, the composition is formulated for injection.

Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the inhibitory nucleic acids can be formulated in liquid solutions, preferably in physiologically compatible buffers such as Hank's solution or Ringer's solution. In addition, the compounds can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, tale or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator may be formulated containing a powder mix of the compound and a suitable powder base such as lactose or starch.

The targeted inhibitory nucleic acids can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compositions can also be formulated as rectal compositions such as suppositories or retention enemas, e.g., containing conventional suppository bases such as cocoa butter or other glycerides.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

### Dosage and Administration

The method and compositions provided herein can be used to sequester and/or inhibit activity of ARG-TCT-4-1 and treat cancer in a subject by administering a therapeutically effective amount of an inhibitory nucleic acid as described herein. In one embodiment, the subject can be a mammal. In another embodiment, the mammal can be a human, although the approach is effective with respect to all mammals.

The appropriate dosage range for an inhibitory nucleic acid depends upon the potency of the particular inhibitory nucleic acid and includes amounts large enough to produce the desired effect, e.g., reduced activity of ARG-TCT-4-1, reduced expression of ARG-TCT-4-1, or treatment of cancer. Although adverse side effects are often associated with anti-cancer agents, the dosage should not be so large as to cause unacceptable or life-threatening adverse side effects. Generally, the dosage will vary with the type of inhibitor, and with the age, condition, and sex of the patient. The dosage can be determined by one of skill in the art and can also be adjusted by the individual physician in the event of any complication.

The effective amount may be based upon, among other things, the size of composition, the biodegradability of the composition, the bioactivity of the composition and the bioavailability of the composition. For example, if the composition does not degrade quickly, is bioavailable and highly active, a smaller amount will be required to be effective. One of skill in the art could routinely perform empirical activity tests for a compound to determine the bioactivity in bioassays and thus determine the effective amount of a given composition or formulation.

Typically, the dosage ranges for a given therapeutic composition is in the range of 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 μg/mL and 30 μg/mL.

As for when the inhibitory nucleic acids, compositions and/or agent is to be administered, one skilled in the art can determine when to administer the inhibitory nucleic acid or composition thereof. The administration can be constant for a certain period of time or periodic and at specific intervals. The compound can be delivered hourly, daily, weekly, monthly, yearly (e.g. in a time release form) or as a one-time delivery. The delivery can be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

As one of skill in the art will appreciate, the dosage of given therapeutic agent (e.g., an inhibitory nucleic acid or composition or formulation thereof) can vary depending upon the dosage form employed and the route of administration utilized. Compositions, methods, and uses that exhibit large therapeutic indices (i.e., the dose ration between toxic and therapeutic effects) are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. A therapeutically effective amount is an amount of an agent that is sufficient to produce a statistically significant, measurable change of a given symptom of a cancer (see “Efficacy Measurement” below). Such effective amounts can also be gauged in clinical trials as well as animal studies for a given agent.

An appropriate therapeutic amount or dose for treating a human subject can be informed by data collected in cell cultures or animal models. In some embodiments, the therapeutic efficacy can be estimated by the ED50 in an animal model (the dose therapeutically effective in 50% of the population) or in a cell cytotoxicity assay (where at least 50% of the cancer cells are killed).

Therapeutic compositions can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

The agents described herein can be administered to a subject in need thereof by any appropriate route which results in an effective treatment in the subject. For example, agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired. Generally, for anti-cancer therapy, the inhibitory nucleic acids or compositions thereof are delivered intravenously or by injection (e.g., into a tumor site, intramuscular, subcutaneous etc.).

“Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the agents described herein, other than directly into a target site, tissue, or organ, such that it enters the subject's circulatory system and, thus, is subject to metabolism and other like processes.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

Combination Therapies: In some embodiments, an inhibitory nucleic acid or composition thereof as described herein is used in combination with at least one additional anti-cancer therapy, such as an anti-cancer agent or chemotherapeutic, X-rays, gamma rays or other sources of radiation to destroy cancer stem cells and/or cancer cells.

Combination therapy using an inhibitor of ARG-TCT-4-1 activity and/or expression and a second anti-cancer treatment (e.g., can comprise administration of the therapeutics to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the combination therapies can be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the combination therapies are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof.

When administered in combination, the anti-cancer agent or drug used in combination with an inhibitory nucleic acid as described herein can be administered in an amount or dose that is lower (e.g., at least 20%, at least 30%, at least 40%, or at least 50% lower) or the same as the amount or dosage of the agent used individually, e.g., as a monotherapy.

Currently available anti-cancer therapies and their dosages, routes of administration and recommended usage are known in the art and have been described in such literature as the Physician's Desk Reference (60th ed., 2017).

### Kits

In one aspect of any of the embodiments, described herein is a pharmaceutical composition, kit, or combination comprising at least one inhibitory nucleic acid as described herein and, optionally, a pharmaceutically acceptable carrier.

A kit is an assemblage of materials or components, including at least one of inhibitory nucleic acids as described herein or a composition thereof. The exact nature of the components configured in the kit depends on its intended purpose. In some embodiments of any of the aspects, the kit is configured particularly for human subjects. In further embodiments, the kit is configured for veterinary applications, treating subjects such as, but not limited to, farm animals, domestic animals, and laboratory animals.

In some embodiments of any of the aspects, a kit includes instructions for use. “Instructions for use” typically include a tangible expression describing the technique to be employed in using the components of the kit to affect a desired outcome in a subject. Still in accordance with the present methods and compositions, “instructions for use” may include a tangible expression describing the preparation of an inhibitory nucleic acid and/or at least one method parameter, such as dosage requirements and administration instructions, and the like, typically for an intended purpose. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of a composition as described herein. The packaging material generally has an external label which indicates the contents and/or purpose of the kit and/or its components.

### Treatment of Cancer

In some embodiments of any of the aspects, the methods described herein relate to treating a subject having or diagnosed as having cancer with a composition as described herein. In certain embodiments, the cancer is characterized by an overexpression of METTL1 and/or ARG-TCT-4-1. In some embodiments, the level of ARG-TCT-4-1 or METTL1 is determined prior to treatment of a cancer using the methods and compositions described herein. Subjects having cancer can be identified by a physician using current methods of diagnosing cancer. Symptoms and/or complications of cancer which characterize these conditions and aid in diagnosis are well known in the art and include but are not limited to, for example, a lump or mass, swelling, skin irritation, pain, redness, fatigue, fever, malaise, limb weakness, decreased range of motion of a limb, weight gain or loss, blood in stool, abdominal pain, abdominal swelling and the like. Tests that may aid in a diagnosis of cancer include, but are not limited to, mammograms, x-rays, MRI, ultrasound, CT-scan, a biopsy, and genetic evaluations. A family history of cancer or exposure to risk factors for cancer (e.g. smoke, radiation, pollutants, BRCA1 mutation, etc.) can also aid in diagnosis of risk of cancer or the presence of cancer.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments, the cancer is one known to be associated with high METTL1 expression. In other embodiments, the cancer can be a sarcoma (e.g., a liposarcoma), a glioblastoma, an adrenocortical carcinoma, a cholangiocarcinoma, a melanoma, a glioma, a diffuse glioma, a mature B cell neoplasm, a non-small cell lung cancer, an esophagogastric adenocarcinoma, a pheochromocytoma, a hepatocellular carcinoma, an endometrial carcinoma, a pancreatic adenocarcinoma, a breast carcinoma, an invasive breast carcinoma, a head and neck squamous cell carcinoma, a bladder urothelial carcinoma, a colorectal adenocarcinoma, an ovarian epithelial tumor, a prostate adenocarcinoma, a cervical squamous cell carcinoma, a renal non-clear cell carcinoma, or a renal clear cell carcinoma.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

The compositions and methods described herein can be administered to a subject having or diagnosed as having cancer. In some embodiments of any of the aspects, the methods described herein comprise administering an effective amount of compositions described herein to a subject in order to alleviate a symptom of a cancer. As used herein, “alleviating a symptom” of a cancer is ameliorating any, or all, conditions or symptoms associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “therapeutically effective amount” or “effective amount” as used herein refers to the amount of at least one inhibitor of ARG-TCT-4-1 needed to alleviate at least one or more symptom of the disease or disorder, and relates to a sufficient amount of pharmacological composition to provide the desired effect. The term “therapeutically effective amount” therefore refers to an amount of at least one inhibitory nucleic acid as described herein that is sufficient to provide a particular anti-cancer effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for tumor growth, among others. The dosage can be determined by a physician and adjusted, as necessary, to suit observed effects of the treatment.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In some embodiments of any of the aspects, the at least one inhibitor of ARG-TCT-4-1 described herein is administered as a monotherapy, e.g., another treatment for the cancer is not administered to the subject.

In some embodiments of any of the aspects, the methods described herein can further comprise administering a second agent and/or treatment to the subject, e.g. as part of a combinatorial therapy.

Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. tumor size or growth rate by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the at least one inhibitory nucleic acid as described herein. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising at least one inhibitory nucleic acid as described herein can be administered over a period of time, such as over a 5 minute, 10 minute, 15 minute, 20 minute, or 25 minute period.

The dosage ranges for the administration of at least one inhibitor of ARG-TCT-4-1, according to the methods described herein depend upon, for example, the form of the at least one inhibitory nucleic acid as described herein, its potency, and the extent to which symptoms, markers, or indicators of a condition described herein are desired to be reduced, for example the percentage reduction desired for tumor size or growth rate. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of the at least one inhibitor of ARG-TCT-4-1 in, e.g. the treatment of a condition described herein, or to induce a response as described herein (e.g. reduction in tumor size and/or growth rate) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. cancer cell survival. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed, e.g. the targeted gene in cancer cells.

In vitro and animal model assays are provided herein which allow the assessment of a given dose of the at least one inhibitory nucleic acid as described herein. By way of non-limiting example, the effects of a dose of the at least inhibitory nucleic acid as described herein can be assessed by cancer cell expression analysis or survival rates. The efficacy of a given dosage combination can also be assessed in an animal model, e.g. a mouse model of cancer.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

### Example 1: METTL1-Mediated m⁷G Modification of Arg-TCT tRNA Drives Oncogenic Transformation

Recent studies of the “epitranscriptome” reveal important roles of different RNA modifications in cancer (Saletore et al., 2012; Torres et al., 2014). For example, METTL3, a N6-methyladenosine (m⁶A) writer that modifies a large subset of mRNAs, is oncogenic when overexpressed (Barbieri et al., 2017; Chen et al., 2018; Choe et al., 2018; Lin et al., 2016; Vu et al., 2017). tRNAs are subject to numerous modifications, including methylation, which controls tRNA folding, stability, and function (Alexandrov et al., 2002; Chou et al., 2017; de Cr6cy-Lagard et al., 2019), and dysregulation is linked to developmental disorders and cancers (Delaunay and Frye, 2019; Kirchner and Ignatova, 2015; Torres et al., 2014). For example, deposition of N5-methylcytosine (m⁵C) by NSUN2 (Blanco et al., 2016; Frye and Watt, 2006) and mcm⁵s2U modification at tRNA nucleotide position 34 play important roles in cancer (Delaunay and Frye, 2019), including resistance to therapy (Rapino et al., 2018). m⁵C and N7-methylguanosine (m⁷G) tRNA modification by NSUN2 and METTL1 (methyltransferase like-1 protein), respectively, has been implicated in 5-fluorouracil (5-FU) sensitivity in HeLa cells (Okamoto et al., 2014). Together, these studies reveal that aberrant RNA modifications can influence tumor initiation and growth. There is also evidence linking dysregulation of individual tRNAs with disease. Overexpression of tRNAi-Met leads to increased metabolic and cell growth rates in immortalized human breast cells (Pavon-Eternod et al., 2009), promotes melanoma metastasis (Birch et al., 2016), and increases tumor growth and vascularization in mice (Clarke et al., 2016). Overexpression of tRNA-Glu(UCC) or tRNA-Arg(CCG) promotes a pro-metastatic state in breast cancer (Goodarzi et al., 2016). Furthermore, deficiency of the Arg-TCT-4-1 isodecoder that is highly expressed in the central nervous system (CNS) causes neurodegeneration and death in mice (Ishimura et al., 2014).

m⁷G at tRNA nucleotide position 46 (m⁷G46) is one of the most prevalent tRNA modifications (Alexandrov et al., 2002, 2005). m⁷G46 is found in the variable loop region of a subset of tRNAs, and the tRNA-Phe structure shows a C13-G22-m⁷G46 base triple interaction that helps stabilize the tertiary structure (Jovine et al., 2000). Mutation of the yeast m⁷G methyltransferase causes rapid tRNA decay (RTD) of hypomodified tRNAs and growth defects under heat stress (Alexandrov et al., 2006). m⁷G tRNA modification is catalyzed by a heterodimeric protein complex (Leulliot et al., 2008) that in mammals comprises the METTL1 and the WD repeat domain 4 (WDR4) and is required for mouse embryonic stem cell self-renewal and differentiation (Lin et al., 2018). METTL1 may also deposit internal m⁷G marks in mRNAs (Chu et al., 2018; Zhang et al., 2019) and microRNA (miRNA) precursors (Pandolfini et al., 2019). METTL1 has not yet been functionally implicated in oncogenesis but is recurrently overexpressed and amplified and was recently found to be upregulated in hepatocellular carcinoma (HCC) and associated with poor outcomes (Tian et al., 2019). Conversely, METTL1 was suggested as a potential tumor suppressor in colon cancer (Liu et al., 2020b), while the overall relevance of METTL1 in cancer remains largely unknown.

Herein, it is shown that the methyltransferase complex METTL1/WDR4 is oncogenic. METTL1 deficiency leads to reduced m⁷G tRNA methylation and expression, global translation and cell cycle defects, and suppression of tumor growth in various xeno-graft models including glioblastoma multiforme (GBM), liposarcoma (LPS), melanoma, and acute myeloid leukemia (AML). Overexpression of METTL1/WDR4 leads to malignant transformation and tumorigenesis.

Mechanistically, it was found that elevated m⁷G tRNA modification upon METTL1 gain of function leads to increased abundance of a tRNA subset, including Arg-TCT-4-1, one of five isodecoder tRNAs (six in human) responsible for decoding AGA codons, and the corresponding increased translation of mRNAs that are enriched in AGA codons, including those related to the cell cycle. Reporter assays show that overexpression of METTL1 or Arg-TCT-4-1 promotes optimal expression of transcripts enriched with AGA codons. The inventors show that tRNA-Arg-TCT-4-1 upregulation phenocopies the METTL1/WDR4 overexpression phenotype and causes malignant transformation and oncogenesis. Accordingly, the inventors identified specific alterations in the proteome upon METTL1 or tRNA-Arg-TCT-4-1 overexpression. This study reveals the functional role and underlying molecular and cellular mechanism of METTL1/WDR4 and m⁷G RNA modification in malignant transformation and highlights its potential as a therapeutic target.

### Results

METTL1 is Amplified and Overexpressed in Human Cancers and is Associated with Poor Patient Survival

METTL1 is located on a region of chromosome 12 (12 q13-14) that is frequently amplified in cancers (Bahr et al., 1999; Wikman et al., 2005). Examination of The Cancer Genome Atlas (TCGA) revealed that METTL1 is amplified in ˜13% of GBM and ˜17% of sarcoma (SARC) patients and commonly amplified in other tumor types (data not shown; see e.g., Figure S1A of Orellana, E, et al. Molecular Cell (2021) 81:3323-333). Within the SARC group, METTL1 amplification is especially common (˜70%) in LPS. There is a positive correlation between METTL1 mRNA expression and amplification of the locus (data not shown, see e.g., Figure S1B of Orellana, E, et al. Molecular Cell (2021) 81:3323-333), and high levels of METTL1 mRNA expression are associated with poor patient survival (data not shown; see e.g., Figures S1C and S1D of Orellana, supra).

Immunohistochemical staining revealed increased METTL 1 and WDR4 protein expression in GBM tumors compared with normal cerebral tissue (data not shown; see e.g., Figure S1E of Orellana, supra). Furthermore, even in cancers without frequent amplification of the locus, METTL1 expression is significantly elevated in most types of cancer compared with normal cells and tissues, including AML (data not shown; see eg., Figures S1F-S1H and S2A of Orellana, supra). Western blot confirmed increased METTL1 protein expression in patient-derived AML samples compared with normal human CD34+ cord blood cells (data not shown, see e.g., Figure S1G of Orellana, supra), and METTL1 is highly expressed in primary murine AML cells compared with their isogenic normal or non-leukemic hematopoietic stem and progenitor cells (HSPCs) (data not shown; see e.g., Figure S1H of Orellana, supra). METTL1 mRNA expression is also positively correlated with elevated expression of WDR4 mRNA (data not shown, see e.g., Figure S2B of Orellana, supra), and METTL1 mRNA and protein levels are positively correlated in various cancer types (data not shown, see e.g., Figure S2C of Orellana, supra). Considering this, the inventors explored METTL1 amplification in human GBM cell lines using copy number data from the Cancer Cell Line Encyclopedia (CCLE) (data not shown; see e.g., Figure S2D of Orellana, supra) and identified and validated LNZ308 (data not shown, see e.g., Figure S2E of Orellana, supra) as a GBM cell line with METTL1 amplification. The inventors also performed the same analysis on a panel of human LPS cell lines and identified LP6, LPS853, and 93T449 with METTL1 amplification (data not shown; see e.g., Figure S2F of Orellana, supra). Western blots show that the level of METTL1 and WDR4 are elevated in these LPS cell lines compared with normal human pre-adipocytes (data not shown; Figure S2G of Orellana, supra). Taken together, the cancer genetics data and expression analyses implicate METTL1 in various cancers.

### METTL1 is Required for Cancer Cell Growth and Tumorigenicity

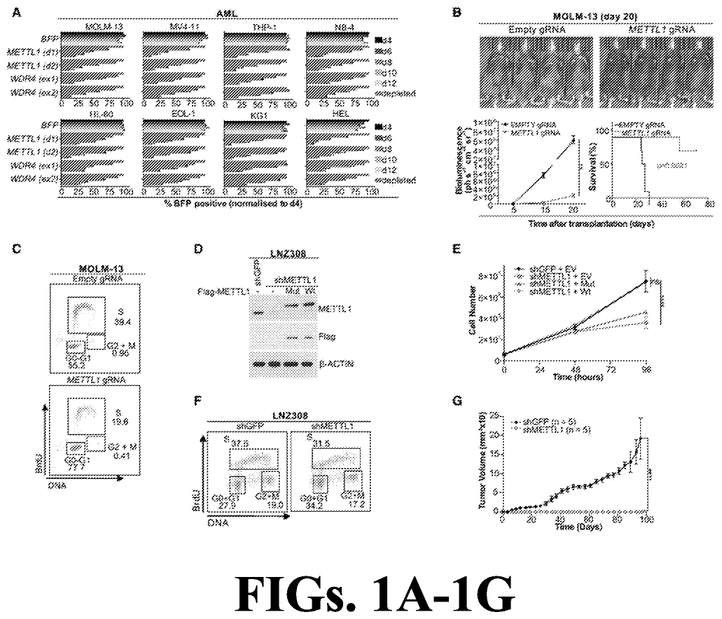

The inventors explored the function of METTL1 in the regulation of cancer cell survival using CRISPR-Cas9-mediated METTL1 and WDR4 knockout (KO). Using a large panel of human cancer cell lines (FIG. 1A; and data not shown; see e.g., Figure S3A of Orellana, supra), it was found that loss of METTL1 or WDR4 is detrimental for overall cancer cell growth. METTL1 deletion also resulted in strong inhibition of cell growth in primary murine AML cells, but not in their isogenic non-leukemic HSPCs (data not shown; see e.g., Figures S3B and S3C of Orellana, supra), and significantly suppressed colony formation of leukemic stem cells but had a negligible effect on the clonogenic potential of normal murine HSPCs (data now shown; see e.g., Figure S3D of Orellana, supra). In line with this observation, normal human CD34+ cord blood cells did not show decreased colony formation efficiency upon METTL1 knockdown (KD) (data not shown; see e.g., Figure S3E of Orellana, supra). To evaluate the effect of Mettl KO in normal hematopoiesis, the inventors performed competitive transplantation experiments, observing no significant difference in the chimerism and the hemopoietic reconstitution between normal HSPCs harboring an empty or a Mettl1 guide RNA (gRNA), while Mettl1 KO was importantly confirmed 8 weeks post-transplantation (data not shown; see e.g., Figure S3F of Orellana, supra). The results indicate that Mettl1 KO has limited or no effect in normal mouse hematopoiesis. The inventors next tested the role of METTL1 in AML progression in vivo by performing mouse xenograft experiments using human MOLM-13-Cas9 AML cells stably expressing a luciferase re-porter. METTL1-KO and control cells (data not shown; see e.g., Figure S4A of Orellana, supra) were transplanted into immunocompromised Rail (Rag2−/−, IL2RG−/−) mice, and in vivo AML expansion was monitored by whole-body measurement of bioluminescence. METTL1 KO results in ablation of cancer progression in vivo and increase of overall mouse survival (FIG. 1B). The inventors next tested whether altered cell cycle could explain these cell growth phenotypes in cell culture (data not shown; see e.g., Figure S4B Orellana, supra) and in vivo (FIG. 1C). Cell cycle analysis revealed that METTL1-KO human AML MOLM-13 and OCI-AML-3 cells have an increased percentage of cells in G1 phase and decreased percentage of cells in S phase, indicating that deletion of METTL1 results in impaired G1/S transition (FIG. 1C; see also e.g., Figure S4B of Orellana, supra). Similar results were obtained in a mouse melanoma model using B16F10 cells with a doxycycline (Dox)-inducible short hairpin RNA (shRNA) against METTL1 (data not shown; see e.g., Figures S4C-S4F of Orellana, supra). The inventors further generated shRNA-mediated stable METTL1 KD in the LNZ308 human GBM cell line (data not shown; see e.g., Figure S5A of Orellana, supra). METTL1 KD led to decreased cell proliferation (data not shown; see e.g., Figure S5B of Orellana, supra), a phenotype that could be rescued by reintroduction of the wild-type (WT) METTL1 cDNA but not of a catalytically inactive mutant METTL1 (FIGS. 1D-1F), indicating that rapid cancer cell proliferation is dependent on m⁷G RNA modification. Biochemical reconstitution and m⁷G activity assays with WT or L160A, D163A version of the METTL1/WDR4 complex confirmed that the activity of the mutant is severely compromised (data not shown; see e.g., Figure S5C of Orellana, supra). Cell cycle analysis revealed that METTL1-deficient LNZ308 cells have an increased percentage of cells in G1 phase (FIG. 1F) without an obvious effect on apoptosis (data not shown; see e.g., Figure S5D of Orellana, supra). METTL1 depletion also resulted in decreased anchorage independent growth of LNZ308 cells (data not shown; see e.g., Figure S5E of Orellana, supra). The inventors next tested the requirement of METTL1 for tumor formation in vivo by performing mouse xeno-graft experiments and measuring tumor formation after subcutaneous transplantation into nude mice. It was found that METTL 1 KD completely suppressed tumorigenesis in vivo (FIG. 1G and data not shown (see e.g., Figure S5F of Orellana, supra)). Similar results were also obtained for the T98G human GBM cell line (see e.g., Figures S5G and S5H of Orellana, supra) and a panel of human LPS cell lines (see e.g., Figures S6A-S6E of Orellana, supra). Overall, these data indicate an important role of METTL1 in controlling cancer cell growth, cell cycle and oncogenicity.

METTL1 Depletion Leads to Decreased Levels of m⁷G-Modified tRNAs and Global Translation Defects

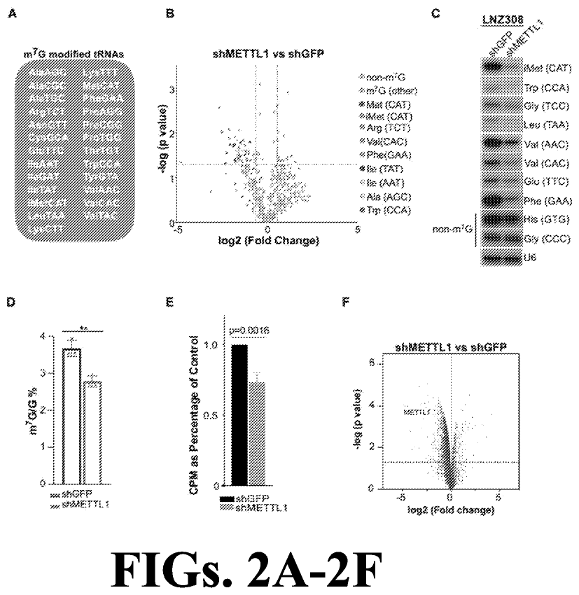

In order to understand the molecular mechanism behind the cell growth defects seen in METTL1-KO cells, the inventors used a recently developed sequencing tool, TRAC-Seq (Lin et al., 2018, 2019) to explore the m⁷G methylome. They identified a subset of 25 tRNAs that are m⁷G modified in LNZ308 cells (FIG. 2A; data not shown (see e.g., Figure S7A of Orellana, supra) and share the RAGGU motif (see e.g., Figure S7B of Orellana, supra). The inventors validated several of these m⁷G tRNAs using NaBH₄/aniline cleavage (data not shown; see e.g., Figure S7C of Orellana, supra) and m⁷G methylated RNA immunoprecipitation (mcRIP) (data not shown; see e.g., Figure S7D of Orellana, supra) followed by Northern blot analyses. The inventors observed a decrease in the abundance of the m⁷G-modified tRNA subset in the METTL1-KD cells compared with the control cells (FIG. 2B; see also e.g., Table S1 of Orellana, supra), further confirmed by Northern blot (FIG. 2C). The inventors next measured m⁷G levels in METTL1-KD RNA samples compared with shGFP control samples using high-performance liquid chromatography with tandem mass spectrometry (HPLC-MS/MS) and observed a decrease in the overall levels of m⁷G/G (FIG. 2D). In line with this observation, there is an overall decrease in the TRAC-seq cleavage scores in shMETTL1 samples compared with the shGFP control that is correlated with the decrease in tRNA abundance in METTL1-KD cells (data not shown; see e.g., Figure S7E of Orellana, supra). Interestingly, the inventors observed a strong decrease in the levels of tRNAi-Met (m⁷G modified), indicating that there could be a global defect in translation initiation. Metabolic labeling using [35S]methionine further supported compromised protein synthesis in METTL1-KD cells (FIG. 2E; data not shown (see e.g., Figure S7F of Orellana, supra). Indeed, SILAC (stable isotope labeling using amino acids in cell culture) proteomics also revealed a widespread decrease in protein synthesis (1,177 significantly down-regulated proteins among 3,047 proteins detected) in METTL1-KD cells compared with shGFP control cells (FIG. 2F; see also Table S2 of Orellana, supra). Taken together, these data indicate that METTL1 deficiency causes loss of m⁷G tRNA modification, decreased stability of hypomodified tRNAs, and global translation defects.

### METTL1/WDR4 Overexpression Drives Oncogenic Transformation and Tumorigenesis

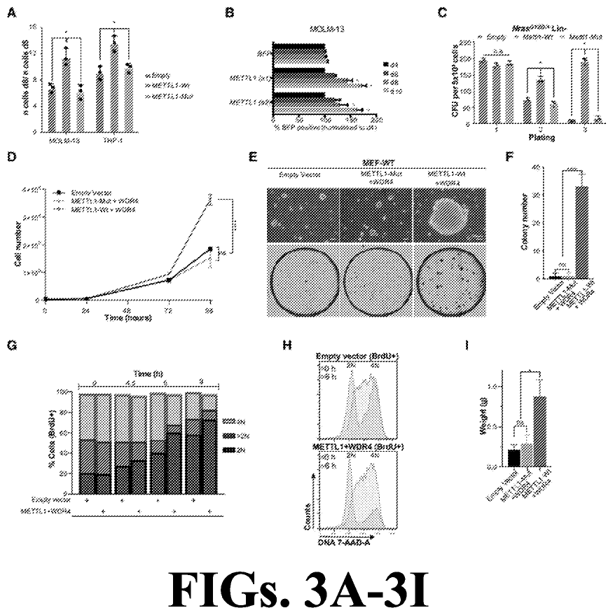

To explore whether METTL1 can act as an oncogene, the inventors next performed gain-of-function experiments in different cellular contexts. It was found that overexpression of METTL1-WT, but not the catalytic mutant version (L160A, D163A: Mut), resulted in increased proliferation of human AML cell lines (FIG. 3A; see also Figure S8A of Orellana, supra). Moreover, CRISPR activation of endogenous METTL1 also results in increased AML cell proliferation (FIG. 3B, see also Figure S8B of Orellana, supra). Ectopic expression of METTL1 (but not the catalytic mutant) is also highly oncogenic in primary murine non-leukemic HSPCs (FIG. 3C). Next, the inventors expressed METTL1 in non-transformed mouse embryonic fibroblasts (MEF-WT cell line with SV40 T antigen). For that purpose, stable clones that overexpress METTL1 and WDR4 were generated (see e.g., Figure S8C of Orellana, supra). The inventors observed increased cell proliferation and ability to form colonies in soft agar in cells overexpressing the WT, but not the catalytic mutant, METTL1 complex (FIG. 3F). Cell cycle analyses of METTL1/WDR4-overexpressing cells revealed that over time, S-phase BrdU-labeled cells (0 h) transitioned from 2N DNA to 4N DNA (G2) content and after undergoing mitosis transitioned to 2N DNA (GI) faster than empty vector (EV) control cells, indicating that METTL1/WDR4 overexpression accelerates cell cycle progression (FIGS. 3G and 3H; see also Figure S8D of Orellana, supra). The inventors also found that METTL1-WT cells are able to form large tumors in vivo within 18 days after transplantation into nude mice (FIG. 3I; see also Figure S8E of Orellana, supra). Taken together, these data indicate that METTL1 is an oncogene.

METTL1/WDR4 Overexpression Leads to Increased Abundance of m7G-Modified tRNAs

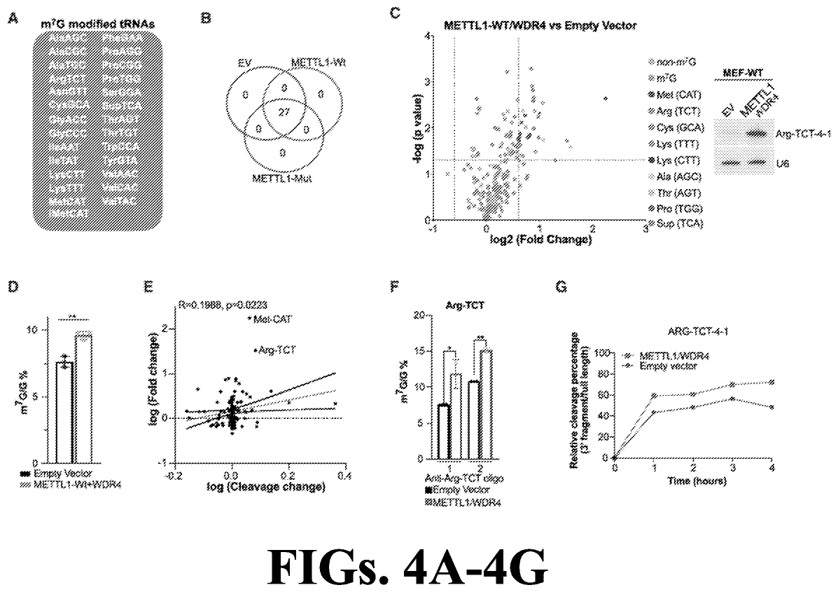

The inventors used TRAC-seq to gain a better understanding of the molecular mechanism that drives the malignant transformation seen in METTL1/WDR4-overexpressing cells. This approach identified a subset of 27 m⁷G-modified tRNAs in MEF-WT cells (FIG. 4A; see also Figure S9A of Orellana, supra). The same tRNA subset was found to be m⁷G modified in negative control (EV), METTL1-WT, and METTL1-Mut (FIG. 4B), and the same RAGGU motif was also enriched among samples (data not shown; see e.g., Figure S9B of Orellana, supra). The inventors validated several of these m⁷G tRNAs using NaBH₄/aniline cleavage (see e.g., Figure S9C of Orellana, supra) and m⁷G mcRIP (data not shown; see e.g., Figure S9D of Orellana, supra) followed by northern blot. The inventors observed increased levels of a subset of m7G-modified tRNAs in the METTL1-WT-overex-pressing samples compared with control, including Arg(TCT), Lys(CTT), Lys(TTT), Pro(TGG), Ala(AGC), and Met(CAT) (FIG. 4C; see also Table S3 of Orellana, supra). The inventors next measured m7G levels in METTL1-WT/WDR4-overexpressing cells compared with EV control samples and observed an increase in the relative m⁷G/G levels (FIG. 4D). Taken together, these results show that ectopic METTL1 expression leads to increased total levels of m7G modification, but according to TRAC-seq, this did not occur at new positions within modified tRNAs or extend to additional tRNAs that are not normally modified. Instead, the inventors find evidence that for particular tRNAs, the proportion that is m⁷G modified is increased upon METTL1 over-expression. This is evidenced by increased NaBH₄/aniline cleavage seen by northern blots (see e.g., Figures S9C and S9G of Orellana, supra), which also manifests as increased stability of tRNAs within the same set (FIG. 4C). Furthermore, for certain tRNAs including Arg-TCT-4-1 and Met-CAT, the increased tRNA abundance is positively correlated with increased methylation (as measured by a change in the NaBH₄/aniline cleavage) in the METTL1-expressing cells (FIG. 4E). To validate this observation, the inventors first measured m⁷G levels in isolated Arg-TCT-4-1 tRNAs (see e.g., Figure S9E of Orellana, supra) from METTL1-WT/WDR4-overexpressing cells compared with EV control samples using HPLC-MS/MS and observed an increase in the relative m⁷G/G levels (FIG. 4F). The inventors also measured m⁷G levels in Arg-TCT using NaBH₄/aniline cleavage in a time-dependent manner to reach saturating conditions and observed that METTL1/WDR4 overexpression leads to increased tRNA cleavage (FIG. 4G; see also Figure S9F of Orellana, supra) and elevated 3′ fragment/full-length ratio (EV=56%, METTL1/WDR4=72%) compared with control. To test whether METTL1 is the enzyme responsible for Arg-TCT m⁷G methylation using an antibody-independent method, the inventors used the meCLICK approach (Mikutis et al., 2020) followed by qRT-PCR in METTL1-WT or METTL1-KD MOLM-13 cells and observed that the level of Arg-TCT was restored in cells with METTL1 downregulation, indicating that the methylation signal of the relevant tRNA was mediated mainly by the catalytic activity of METTL 1 (see e.g., Figure S9G of Orellana, supra). This effect was not observed when meCLICK was applied to the non-m⁷G-modified tRNA His-GTG. Taken together, these data indicate that METTL1/WDR4 overexpression leads to increased m⁷G methylation of a subset of tRNA substrates, particularly Arg-TCT.

METTL1/WDR4 Overexpression Leads to Altered mRNA Translation

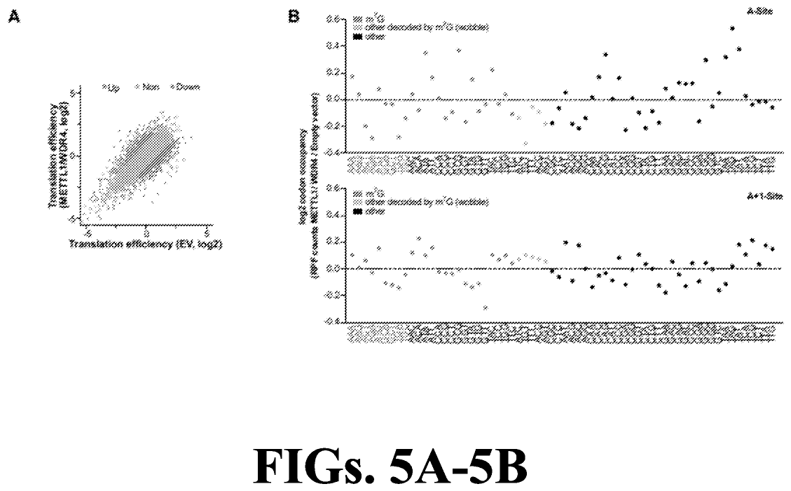

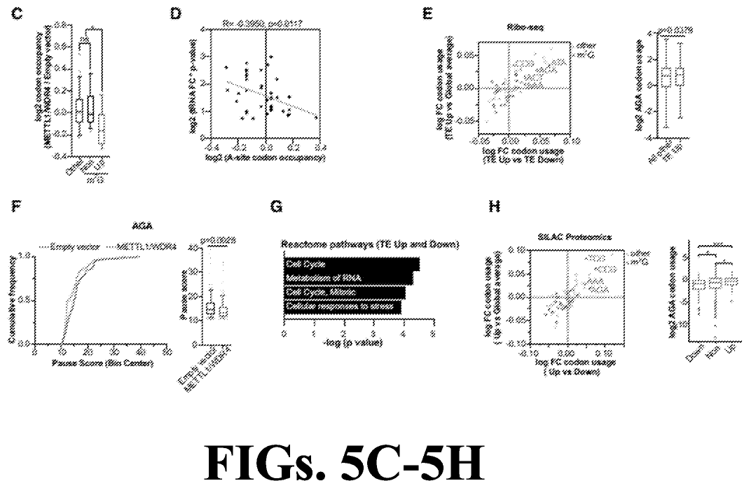

Considering the changes in tRNA abundance upon METTL1/WDR4 overexpression, the inventors reasoned that this could affect mRNA translation. As metabolic labeling revealed no significant global change in translation between samples (see e.g., Figures S10A and S10B of Orellana, supra), the inventors next used ribosome footprinting (Ribo-seq) to evaluate codon use and relative translation efficiencies (TEs) in cells overexpressing METTL1/WDR4 and control cells. This revealed>2-fold changes in the TEs of 864 mRNAs, with similar numbers of mRNAs with increased or decreased TEs (FIG. 5A; see also Table S4 of Orellana, supra). Next, it was asked if there are differences in the overall codon use between METTL1/WDR4-expressing and control cells using the CONCUR pipeline (Frye and Bornelov, 2021). Increased tRNA abundance and function is expected to lead to reduced ribosome dwell time at the cognate codon (Nedialkova and Leidel, 2015; Wu et al., 2019; Zinshteyn and Gilbert, 2013). Comparison of codon occupancy revealed that METTL1/WDR4 over-expression results in decreased ribosome interaction at m7G-tRNA-dependent codons in the charged tRNA binding site (A site), including AGA, ACT, TGC, and other m7G-modified tRNAs, and as a control there is little change in the A+1 site (FIG. 5B). This effect was more pronounced in the codon subset that is recognized by tRNAs with increased abundance upon METTL1 overexpression (FIG. 4C). Increased tRNA abundance is significantly associated with decreased A site occupancy (FIGS. 5C and 5D). Ribo-seq data can also be interpreted as a proxy for overall TE for any particular gene when the ribosome-protected fragment (RPF) coverage across the entire mRNA coding sequence (CDS) (open reading frame) relative to the mRNA expression is analyzed. Considering this, the inventors compared the codon use in the CDS (number of observed codons/number of expected codons [genome average]—normalized by length) between genes with increased TEs and both genes with decreased TEs and the global average (entire dataset) and found that several m⁷G-decoded codons are enriched in TE-up compared with both TE-down and the global average (FIG. 5E). This analysis revealed a significant enrichment of AGA, ACT, and AAA codons in the mRNAs with increased TE due to METTL1/WDR4 expression (FIG. 3G). These codons also show decreased A site occupancy (FIG. 5E), from which AGA shows the most pronounced decrease. As AGA codons showed decreased ribosome occupancy, and genes enriched with AGA codons have higher TEs in METTL1-overexpressing cells compared with control, it was next tested if AGA codons could lead to ribosome pausing. To test this, the inventors measured the ribosome pauses (RPF density at a particular codon relative to background density) at AGA codons and found that control cells have more pauses (170 versus 99) and significantly higher AGA pause scores compared with METTL1-expressing cells (FIG. 5F). This result supports the hypothesis that changes in TE are related to an enrichment of AGA m⁷G tRNA-decoded codons in mRNAs that are decoded with more efficiency in METTL1-overexpressing cells. Gene Ontology analysis revealed that mRNAs with altered TEs in the METTL1/WDR4-expressing cells function in cell cycle progression, providing further support for the involvement of METTL1-mediated m⁷G tRNA modification and corresponding changes in TE in the regulation of the cell growth phenotypes observed (FIG. 5G). Furthermore, AGA was the highest enriched codon in the list of cell cycle mRNAs with increased TEs versus both decreased TEs and global average (see e.g., Figure S10C of Orellana, supra), indicating that the increased abundance of the corresponding Arg-TCT tRNA can play a role in the cell cycle and proliferation changes caused by METTL1/WDR4 expression (FIG. 5G). Moreover, when the inventors examined AGA codon use and ranked all (19,859) mRNAs in the human genome on the basis of the number of AGA codons they contain, the inventors found that (1) AGA is not a rare codon among the six possible codons for arginine (see e.g., Figure S10D of Orellana, supra), (2) there is a small number of genes with many AGA codons (see e.g., Figure S10D of Orellana, supra), and (3) Gene Ontology of the top 1% of mRNAs (170) containing the most AGA codons (ranging in number from 47 to 717 AGA codons within the open reading frame) is significantly enriched for cell cycle mRNAs (see e.g., Figure S10E of Orellana, supra), thereby providing additional evidence that Arg-TCT, particularly the Arg-TCT-4-1 isodecoder, is especially important for the mRNA translation of certain genes involved in cell cycle control. This connection is specific to AGA, as the same analysis for other Arg codons did not identify an enrichment for cell cycle genes (data not shown). Overall, these results identify a critical role of the METTL1 and the m⁷G tRNA methylome in regulating the translation of several cell-cycle-related genes and identify a small subset of tRNAs including Arg-TCT-4-1 that can be especially important for altered TE and METTL1-mediated oncogenesis.

METTL1/WDR4 Overexpression Leads to Upregulation of Proteins Enriched with AGA Codons

SILAC-based quantitative proteomics was used to assess global protein expression in METTL1/WDR4-overexpressing cells compared with control. Using this method, 4,305 proteins were identified, with a false discovery rate of <1%. Of these, 518 proteins that were significantly upregulated and 744 downregulated in METTL1/WDR4-overexpressing cells compared with control (p<0.05, moderated t test, and fold change [FC]>1.2) (see e.g., Figure S10F; Table S5 of Orellana et al., supra). It was next examined if the changes in protein expression are due to enhanced use of any particular codon. For this purpose, the inventors compared the codon use between upregulated proteins and both downregulated proteins and the global average (entire dataset) and found that several m⁷G-decoded codons are enriched in FC-up compared to both FC-down and the global average (FIG. 5H). This analysis revealed a significant enrichment of AGA and AAA codons in the upregulated proteins due to METTL1/WDR4 expression (FIG. 5H). In line with this, it was found that mRNAs encoding the upregulated proteins show higher AGA use compared with unchanged and downregulated proteins (FIG. 5H). These results support previous findings that genes enriched with AGA codons are preferentially upregulated in METTL1/WDR4-overexpressing cells. Taken together, these results indicate that Arg-TCT, an m⁷G-modified upregulated tRNA that decodes AGA, is responsible for some of the changes in protein synthesis upon METTL1/WDR4 overexpression (FIG. 4C).

Altered tRNA Expression in Human Tumors

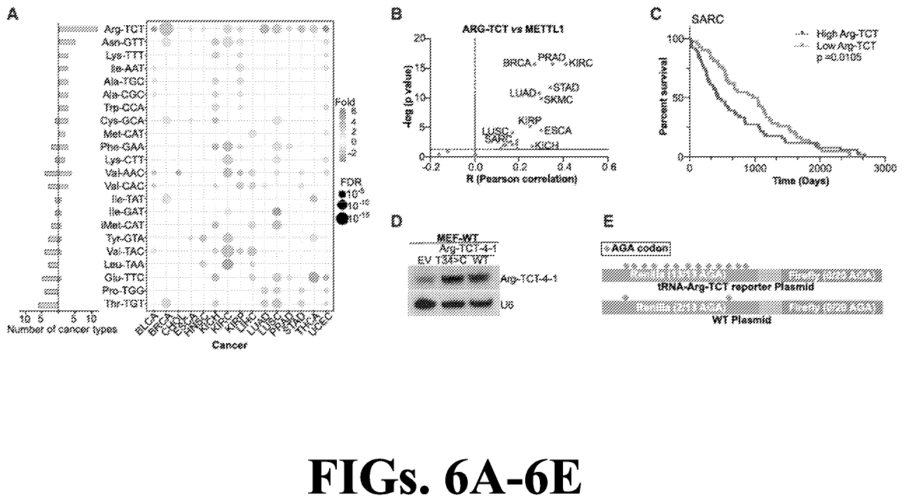

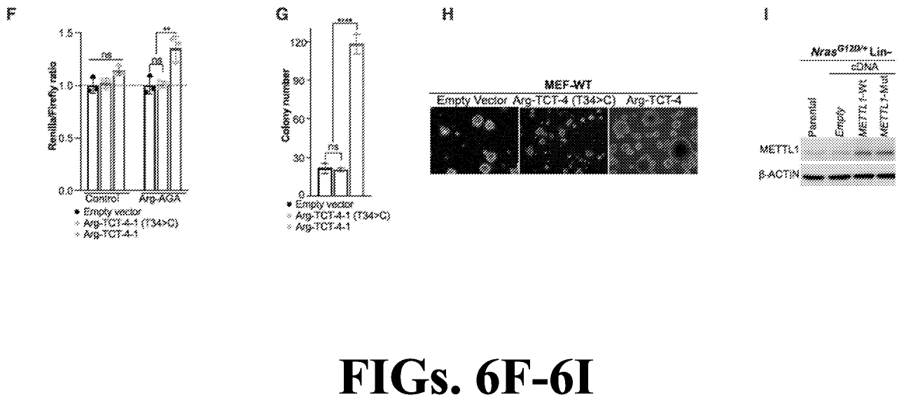

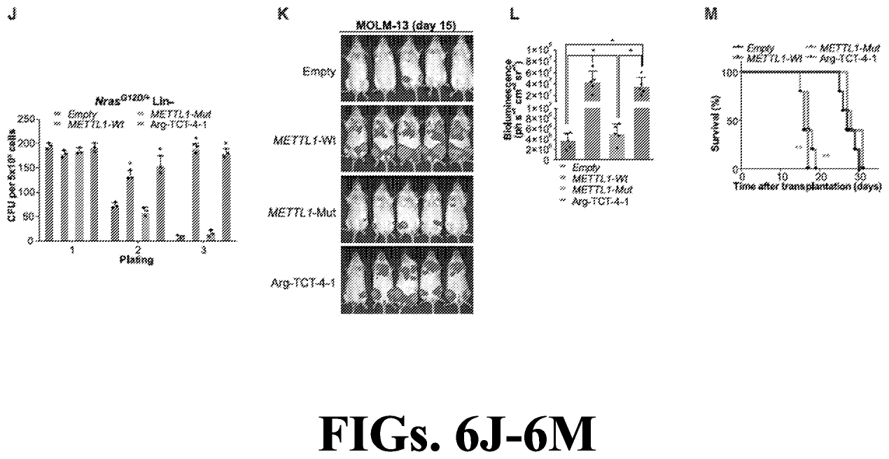

The inventors next comprehensively analyzed the expression profiles of tRNAs from TCGA by re-analyzing miRNA sequencing (miRNA-seq) data across 15 cancer types, including all samples with small RNA sequencing data for both tumor and normal tissues (Zhang et al., 2018). This analysis identified that 22 of the subset of 25 m⁷G-modified tRNAs are dysregulated across different tumor types (FIG. 6A; see also Table S6 of Orellana, et al., supra). Most strikingly, Arg-TCT was the tRNA that is most highly upregulated in tumors compared with normal tissue, and this observation is true for almost all of the 15 cancer types analyzed (FIG. 6A; see also Table S6 of Orellana, et al., supra). Furthermore, this elevated Arg-TCT level in tumors correlates with METTL1 expression in the majority (17 of 22) of cancer types analyzed (FIG. 6B; see also Figure S11A of Orellana, et al., supra), whereas the expression of other tRNAs, including Thr-TGT (which did not increase in METTL1-overexpressing fibroblasts) was found not to correlate with METTL1 expression in SARC (data not shown; see Figure S 11B of Orellana, et al., supra) or other tumor samples (data not shown). Next, the inventors analyzed the individual isodecoder expression profiles of tRNA-Arg-TCT from TCGA and identified that Arg-TCT-4-1 is one of the most dysregulated Arg-TCT isodecoders across multiple different tumor types (see e.g., Figure S11C of Orellana, et al., supra). Moreover, the inventors found evidence of increased Arg-TCT-4-1 abundance (see e.g., Figure S11D of Orellana, et al., supra) in LPS cell lines with METTL1 amplification (see e.g., Figure S2F of Orellana, et al., supra) compared with cells with no METTL1 amplification. This elevated expression of Arg-TCT was associated with poor patient survival in SARC and several other types of cancer (FIG. 6C and data not shown). Taken together, these data indicate that increased METTL1 expression in tumors correlates well with increased abundance of certain m⁷G-modified tRNAs and that many of the same changes in tRNA expression observed in METTL1-expressing fibroblasts are recapitulated in clinical samples, including increased Arg-TCT, Lys-TTT, Ile-AAT, Cys-GCA, and Met-CAT expression (FIG. 4C and FIG. 6A). This analysis also highlights Arg-TCT-4-1 as a tRNA that can mediate some of the oncogenic effects of METTL1 in several cancers.

Oncogenic Role of Arg-TCT-4-1 tRNA

Considering that Arg-TCT-4-1 is (1) one of the most upregulated tRNAs in METTL1-overexpressing cells, (2) the most differentially expressed m7G-modified tRNA in tumors compared with normal tissue, and (3) correlated with METTL1 expression in tumors and with poor patient survival, as well as a previous finding that the corresponding codon (AGA) is highly enriched in the mRNAs with increased TEs in METTL1/WDR4-overexpressing cells, the inventors next explored the effect of overexpression of this tRNA in translation and its possible role in oncogenic transformation. To test this, tRNA expression vectors were engineered by subcloning genomic sequence spanning the tRNA including 300 nt upstream and 100 nt downstream sequence (containing the endogenous PolIII promoter, upstream leader, tRNA, and downstream trailer sequences) to a lentivirus vector without a promoter. Ectopic expression of Arg-TCT-4-1 (FIG. 6D) and its aminoacylation status (see e.g., Figure S11E of Orellana, et al., supra) was confirmed by northern blotting. The inventors next tested the functionality of the overexpressed tRNA and explored whether levels of Arg-TCT-4-1 is limiting for maximal TE in untransformed fibroblasts. To this end, a dual luciferase vector was used in which the inventors converted all of the 13 arginine codons in the Renilla luciferase into AGA codons to generate an Arg-TCT reporter. Because only 2 of 13 arginine codons are AGA in the unmodified Renilla luciferase (WT), it was expected that overexpression of Arg-TCT-4-1 tRNA to have a greater effect on expression of the Renilla luciferase from the Arg-TCT reporter than for the WT reporter (FIG. 6E). In both cases, the Renilla luciferase was normalized to firefly, as none of the 20 arginine codons in firefly is AGA. It was found that expression of Arg-TCT-4-1 tRNA specifically enhanced expression of the Arg-TCT reporter and this effect was dependent on overexpression of Arg-TCT-4-1, as expression of a control Arg-TCT-4-1 T34>C mutant tRNA (CCT anticodon) had no effect (FIG. 6F). Next, it was tested whether upregulation of Arg-TCT-4-1 can promote oncogenic transformation of MEFs as measured by anchorage independent growth. The inventors found that overexpression of Arg-TCT-4-1 was able to enhance the number colonies in this assay (FIG. 6G and FIG. 6H), but not the control Arg-TCT-4-1 T34>C tRNA, indicating that at least some of the oncogenic effects of METTL 1 are mediated through its increased expression of Arg-TCT-4-1 and its AGA decoding function. Furthermore, it was found that Arg-TCT-4-1 overexpression is able to phenocopy METTL1 overexpression and is also highly oncogenic in primary murine non-leukemic Nras^{G1ZD+} HSPCs (FIG. 6I and FIG. 6J). Interestingly, the inventors found that Arg-TCT is highly expressed in two different primary murine AML models compared with their normal or isogenic non-leukemic HSPCs (see e.g., Figure S11F of Orellana, et al., supra). Notably, ectopic overexpression of Arg-TCT-4-1 in human AML MOLM-13 cells resulted in increased cancer progression in vivo (FIG. 6K and FIG. 6L) and phenocopied METTL1-WT overexpression in overall survival (FIG. 6M). In summary, the inventors identify Arg-TCT-4-1 as a key mediator of oncogenic transformation. Arg-TCT-4-1 overexpression recapitulates METTL1/WDR4-mediated proteome changes.

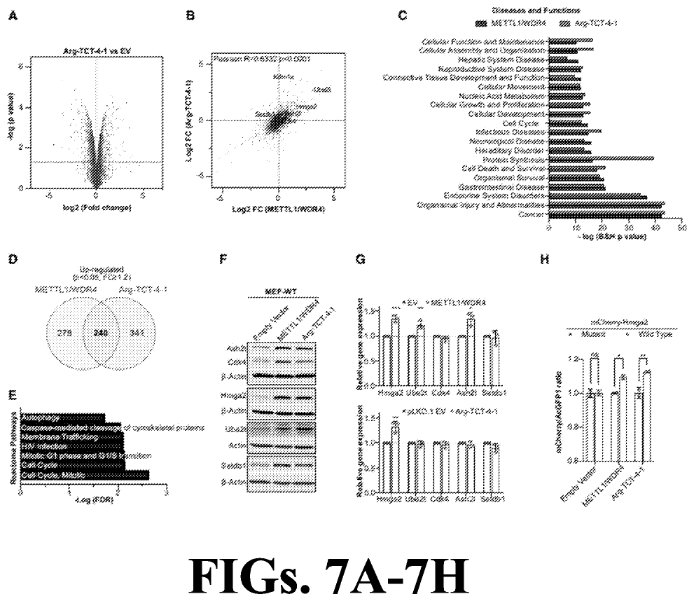

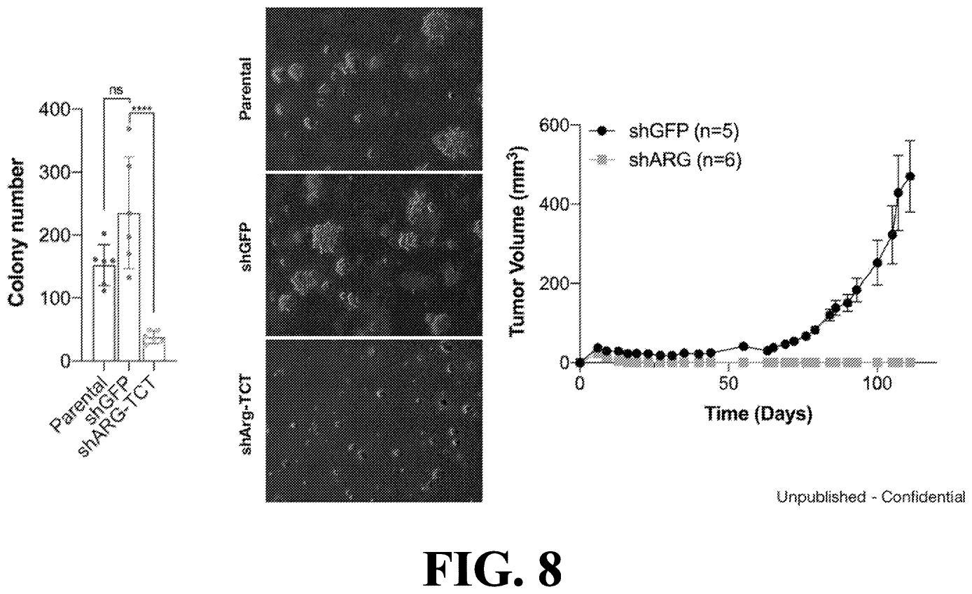

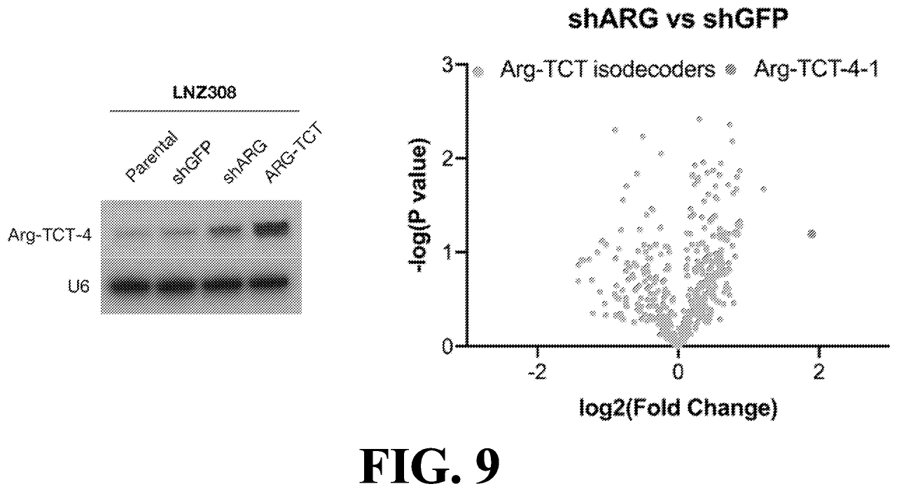

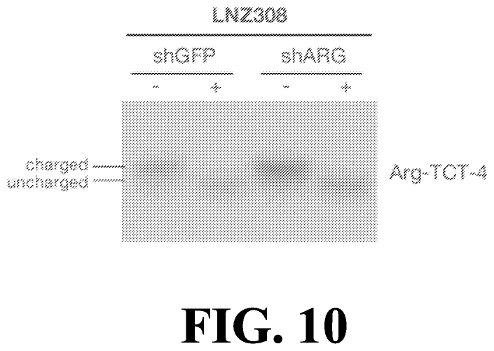

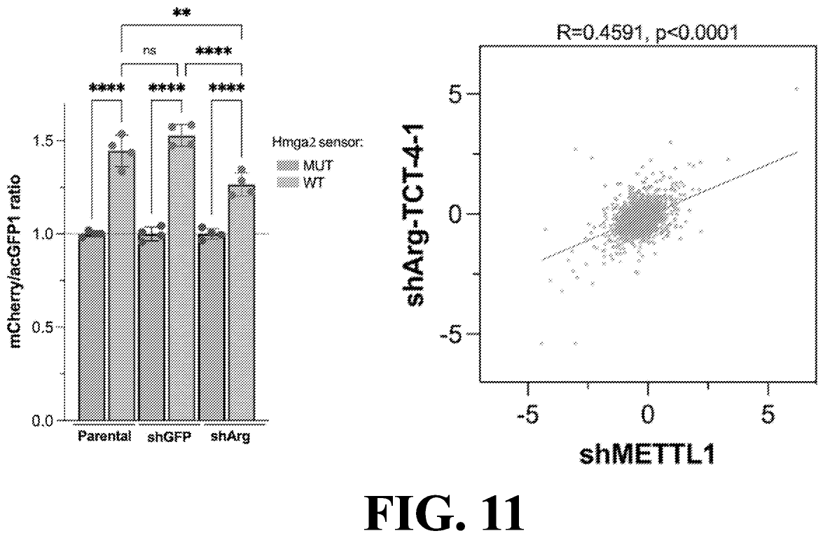

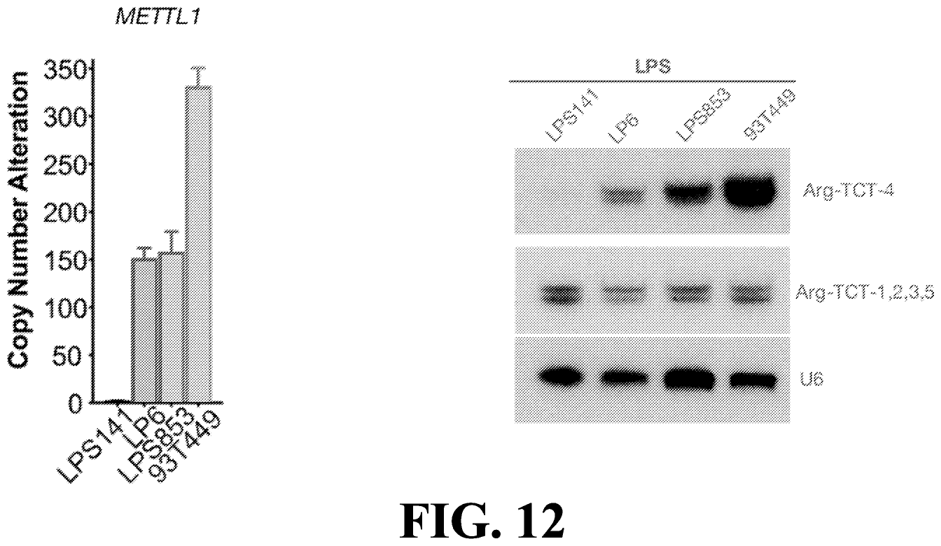

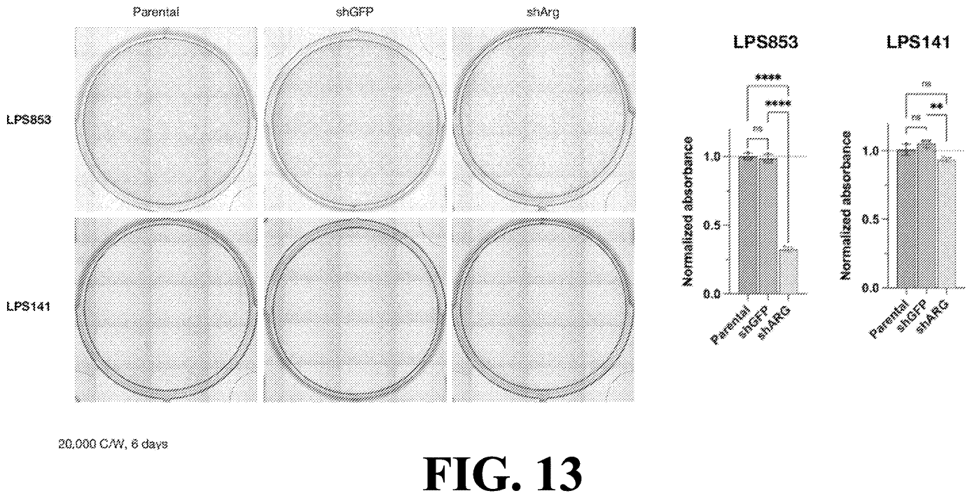

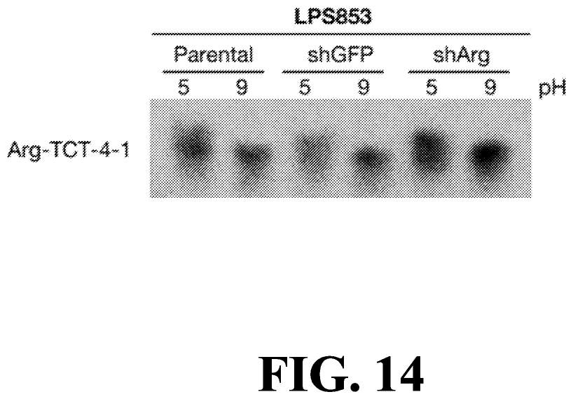

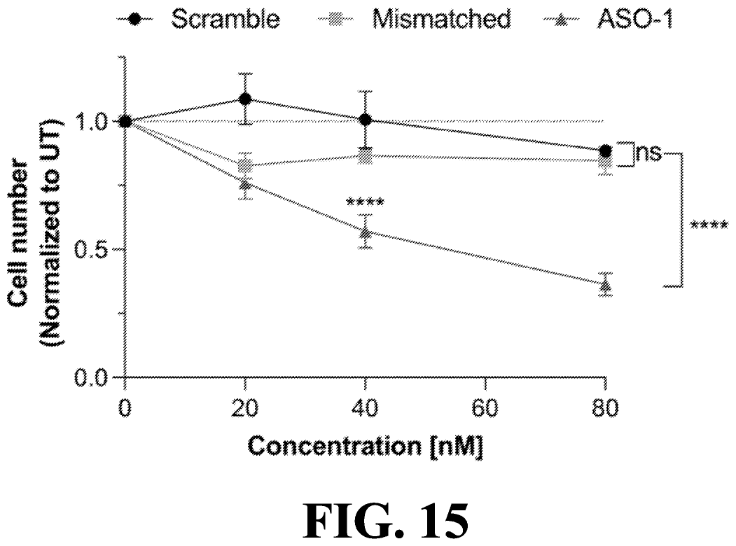

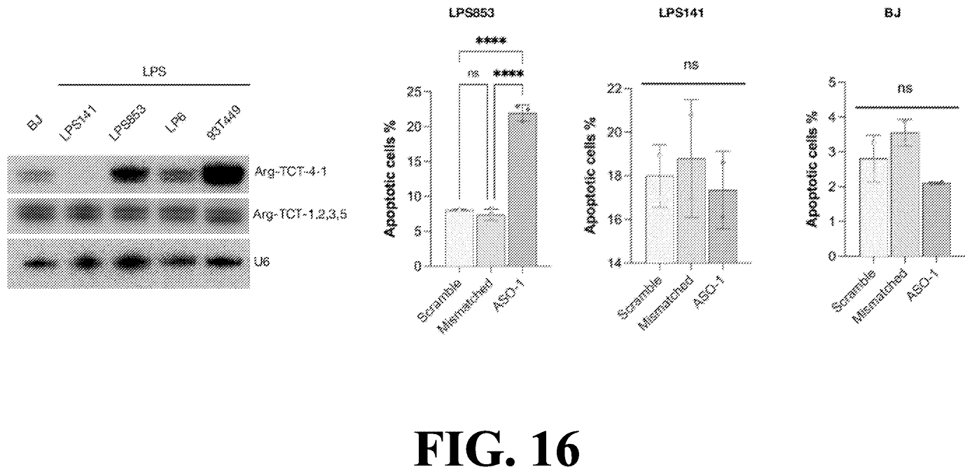

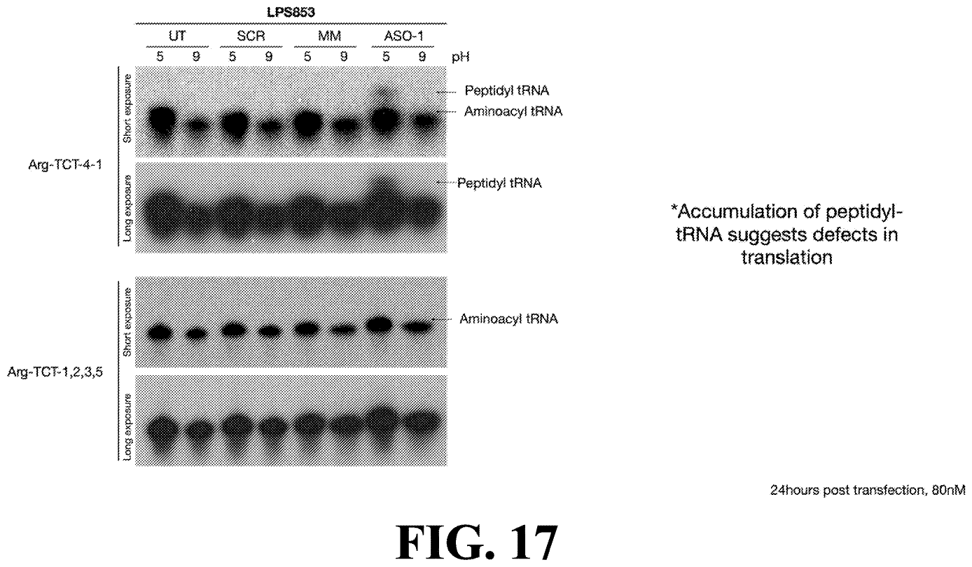

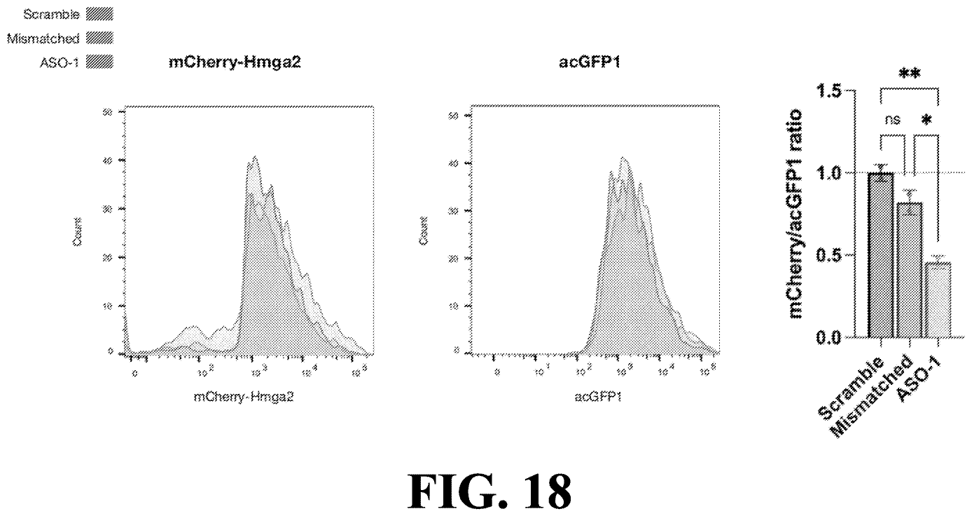

SILAC-based quantitative proteomic analysis was used to assess global protein expression in Arg-TCT-4-1-overexpressing cells compared with control. A total of 4,142 proteins were identified, with a false discovery rate of <1%. The inventors identified 581 significantly upregulated and 709 significantly downregulated proteins in Arg-TCT-4-1-overexpressing cells compared with control (p<0.05, moderated t test, and FC>1.2) (FIG. 7A; see also Table S7 of Orellana, et al., supra). Comparing the changes in protein expression observed in METTL1/WDR4-expressing cells with Arg-TCT-4-1-overexpressing cells revealed a highly significant positive correlation (R=0.6332, p<0.0001) between the two proteomic sets (FIG. 7B). Furthermore, Gene Ontology analysis of both sets showed similar enrichment in diseases and biological functions, with “cancer” identified as the most mutually significant enriched term (FIG. 7C). Next, the inventors looked at the overlap of the upregulated proteins (p<0.05, FC>1.2) in both datasets and found 240 proteins in common, representing 46% and 41% of the pool of proteins upregulated in METTL1/WDR4- and Arg-TCT-4-1-expressing cells, respectively (FIG. 7D). Gene Ontology analysis revealed that commonly upregulated proteins function in the regulation of cell cycle progression (FIG. 7E), similar to what was found by Ribo-seq analysis (FIG. 5G). The inventors validated the expression changes of several proteins that were upregulated in both datasets and have been implicated in oncogenesis in various cancers. This analysis included genes with different levels of enrichment of AGA codons such as Cdk4 (AGA per 1K: mouse, 9.87; human, 13.16), Hmga2 (AGA per 1K: mouse, 38.46; human, 40.54), Ash2l (AGA per 1K: mouse, 12.82; human, 7.95), Setdb1 (AGA per 1K: mouse, 9.93; human, 7.74), and Ube2t (AGA per 1K: mouse, 19.51; human, 30.30). The inventors observed increased protein levels at the western blot level (FIG. 7F) and minimal or no change at the transcript level using qRT-PCR (FIG. 7G). Moreover, it was also found that HMGA2 and KDM1a (AGA per 1K: mouse, 6.86; human, 7.98) proteins are strongly upregulated in human AML MOLM-13 cells expressing WT METTL1 and Arg-TCT-4-1, but not in EV control or mutant METTL1 (see e.g., Figure S12A of Orellana, et al., supra), while no significant change at the transcript level was detected (see e.g., Figure S12B of Orellana, et al., supra). The inventors then asked if the differences in the content of AGA codons could be responsible for the changes in protein abundance mediated by METTL1/WDR4 or Arg-TCT-4-1 overexpression. For this purpose, a fluorescent reporter was generated on the basis of fusion proteins of Hmga2-WT and codon modified Hmga2-MUT (all AGA codons changed to CGC) to mCherry in a bidirectional promoter vector that also expresses acGFP1 as an internal control (0 AGA codons) (see e.g., Figure S12C of Orellana, et al., supra). The reason the inventors chose this endogenous gene is that Hmga2 shows a large number of AGA codons (AGA per 1K: mouse, 38.46; human, 40.54), it is one of the most dysregulated proteins in the METTL1/WDR4 and Arg-TCT-4-1 proteomics dataset, and HMGA2 has been frequently involved in cancer. This fluorescent reporter shows that METTL1/WDR4 and Arg-TCT-4-1 overexpression leads to high mCherry/acGFP1 levels when it is fused to Hmga2 WT compared with the mutant version of Hmga2 that lacks AGA codons (AGA were mutagenized to CGC) (FIG. 7H; see also Figure S12D of Orellana, et al., supra). Taken together, these data show that the changes in protein synthesis observed in METTL1/WDR4-expressing cells can be recapitulated by overexpression of Arg-TCT-4-1.

Here the inventors identify METTL1 as a potent new oncogene that is frequently amplified and/or overexpressed in many human cancers. Remarkably, it was also found that an individual m⁷G-modified tRNA, Arg-TCT-4-1, is largely responsible for METTL1 oncogenicity. METTL1 KD or deletion strongly suppresses cancer cell proliferation and cell cycle progression, blocks tumor growth in a plethora of cancer models, leads to a substantial decrease in the levels of tRNAs that harbor the m⁷G modification, and globally decreases mRNA translation, thereby highlighting METTL1 as a possible therapeutic target in multiple cancer types. To understand the role of METTL1-mediated m⁷G RNA methylation in the control of cell growth and to recapitulate METTL1 amplification and upregulation in human cancers, the inventors performed METTL1 gain-of-function experiments in different cellular contexts. Overexpression of the METTL1/WDR4 complex in MEFs leads to malignant transformation, including increased cell proliferation, accelerated cell cycle progression, enhanced colony formation, and in vivo tumor formation. Overexpression of WT methyltransferase complex, but not catalytic dead mutant, leads to increased abundance of a small subset of tRNAs that are m⁷G modified. METTL1 overexpression did not result in a global change in translation but rather affected a relatively small subset of mRNAs. Ribo-seq showed that overexpression of the WT methyltransferase complex causes changes in translation of genes involved in cell cycle that are enriched in AGA codons. Ectopic Arg-TCT-4-1 expression enhances MEF colony formation in soft agar and phenocopies the effect of METTL1 expression in non-leukemic mouse HSPCs and in human AML cells. Moreover, SILAC-based proteomics further corroborated the involvement of METTL1 and Arg-TCT-4-1 in malignant transformation and the selective upregulation of genes enriched with AGA codons that are involved in cell cycle. Overall, these results reveal the underlying molecular and cellular mechanism of METTL1 oncogenicity that involves increased mRNA translation of a subset of cell cycle regulatory genes that are enriched in AGA codons.

Altered tRNA expression has been generally regarded as a consequence of the high proliferative and metabolic state of cancer cells (Goodarzi et al., 2016; Santos et al., 2019); however, it is becoming increasingly evident that tRNA dysregulation can play more active roles in tumorigenesis (Goodarzi et al., 2016). Moreover, it is emerging that the relative expression of individual tRNAs is highly variable between different normal human (Dittmar et al., 2006) or mouse (Pinkard et al., 2020) tissues, and altered tRNA expression is associated with cell proliferative states (Gingold et al., 2014). The inventors found that increased expression of a single tRNA, Arg-TCT-4-1, can promote cancer initiation and that this relies on its AGA decoding function. Furthermore, work performed in yeast has shown that impairment of wobble modifications mediated by Trm9 in tRNA-Arg-TCT leads to changes in protein expression in genes enriched in AGA codons that are involved in cell cycle and DNA damage control; thus providing further support that Arg-TCT is a key mediator of cell cycle regulation (Begley et al., 2007; Deng et al., 2015). The inventors conclude that METTL1-mediated malignant transformation is due to altered m⁷G modification and abundance of certain tRNAs, in particular Arg-TCT-4-1, which leads to a remodeling of the mRNA “translatome” with increased translation of mRNAs enriched in the respective AGA codon, including a group of cell cycle regulators. This study highlights the potential of METTL1 as a druggable target against cancer.

### Example 2: Methods and Materials

### Cell Lines

Primary mouse embryonic fibroblast with SV40 T antigen (MEF-WT, CRL2991) human T98G (male) (CRL1690) and human glioblastoma LNZ308 cells (male) (CRL11543) were purchased from ATCC. LP6 (sex unspecified) (Snyder et al., 2009) was a gift from Eric Snyder, LPS141 (sex unspecified) (Snyder et al., 2009) and LPS853 (sex unspecified) (Ou et al., 2015) were gifts from Jonathan Fletcher, and 93T449 (female) (Pedeutour et al., 1999) was a gift from Florence Pedeutour. Human white pre-adipocyte cells (sex unspecified) (C-12735) were purchased from Promocell and cultured in preadipocyte growth media according to manufacturer's instructions. Passage 2 was used for experiments. MEF-WT, T98G and LNZ308 were cultured in DMEM supplemented with 10% FBS and 1× penicillin/streptomycin. LPS141 and 93T449 cells were cultured in RPMI 1640 medium supplemented with 15% FBS and 1× penicillin/streptomycin. LPS853 cells were cultured in IMDM medium supplemented with 15% FBS and 1× penicillin/streptomycin. LP6 cells were cultured in DMEM/F12 medium supplemented with 10% FBS, 1% Glutamax and 1× penicillin/streptomycin. All cell lines were cultured in the presence of 5% CO2 at 37° C. MOLM-13 (male), MV4-11 (male), THP-1 (male), NOMO-1 (female), EOL-1 (male), HEL (male), HL-60 (female), MEC-1 (male), MEC-2 (male), JURKAT (male), SU-DHL-5 (female), BxPC3 (female) and SU86.86 (female) were cultured in RPMI1640 (Invitrogen, 21875091) supplemented with 10% FBS (Invitrogen, 16000044) and 1% penicillin/streptomycin/glutamine. Peer was cultured in RPMI1640 (Invitrogen, 21875091) supplemented with 20% FBS (Invitrogen, 16000044) and 1% penicillin/streptomycin/glutamine. NB-4 (female) and KG-1 (male) were cultured in IMDM (Invitrogen, 12440061) supplemented with 10% FBS (Invitrogen, 16000044) and 1% penicillin/streptomycin/glutamine. 293T (female), B16F10 (mouse, female), PANC-1 (male) and PA-TU-8988T (female) cells were cultured in DMEM (Invitrogen, 31600083) supplemented with 10% FBS (Invitrogen, 16000044) and 1% penicillin/streptomycin/glutamine. The following cancer cell lines were obtained from the Sanger Institute Cancer Cell Collection (available on the world wide web at cellmodelpassports.sanger.ac.uk) and negative for mycoplasma contamination: MOLM-13, MV4-11, THP-1, NOMO-1, EOL-1, HEL, HL-60, MEC-1, MEC-2, JURKAT, SU-DHL-5, BxPC3, SU86.86, 293T, B16F10, PANC-1 and PA-TU-8988T. AML patient and cord-blood-derived CD34+ cell samples (independent of sex) were obtained with informed consent under local ethical approval (REC 07-MRE05-44). Human cell lines employed are not listed in the cross-contaminated or misidentified cell lines database curated by the International Cell Line Authentication Committee (ICLAC).

### Animal Subjects

4-6 weeks old female NU/J (Nude) immunodeficient mice (Jackson Laboratory #002019) were used for subcutaneous injections. Female 6- to 10-week-old NSGS mice or Rag2/IL2RG/mice were used for tail-vein or subcutaneous injections. Flt3^{ITD/+} mice² were kindly provided by Gary Gilliland and crossed with Rosa26^{cas9/+} mice. 6- to 10-week-old female Rosa26^{cas9/+},Nras^{G12D+}, Flt3^{TD/+}, Rosa26^{cas9/+}, or moribund Npm1^{flux-ct/+}, Flt3^{ITD/+}, Npm1^{flux-ct/+}, Nras^{G12D+} mice were used for bone marrow isolation of hematopoietic progenitors.

### Plasmid Construction

For WDR4 overexpression, the full-length WDR4 cDNA was first PCR amplified from the 293T cDNA with primers that contain the XhoI (forward primer) and BgIII (reverse primer) sites. A Kozak sequence and 6×His tag were incorporated into the forward primer. Then the PCR product was gel purified and digested with XhoI and BgIII at 37° C. for 1 hour. After that the digested PCR product was purified again and ligated into the XhoI/BgIII cut pBabe-Puro vector. WDR4 was cut and subcloned into a pBabe-Neo vector using EcoRI and SalI restriction sites. For METTL1, overexpression previously generated plasmids expressing METTL1 wild-type and catalytic dead mutant (aa160-163, LFPD to AFPA) were used (Lin et al., 2018). For METTL1 rescue experiments, the inventors generated a plasmid with a mutant shRNA binding site using site directed mutagenesis. Methyltransferase plasmids for recombinant protein expression were generated using the pETDuet-1 expression plasmid. 6×His-WDR4 was cloned into pETDuet-1 using BamHI and SalI RE sites. Next, wild-type and catalytic dead mutant Flag-METTL1 (L160A, D163A) proteins were cloned into pETDuet-1-WDR4 plasmid using NdeI and XhoI RE sites. To overexpress individual tRNAs, the inventors first removed the U6 promoter from a pLKO.1 lentivirus vector using site directed mutagenesis and at the same time introduced a SalI RE site in the multiple cloning site (pLKO.1-puro-AU6). The inventors PCR amplified the genomic sequence spanning the tRNA including 300 nt upstream and 100 nt downstream sequence (containing the PolIII promoter, upstream leader, tRNA, and downstream trailer sequences) and inserted MluI and SalI RE sites in the flanking regions of the amplicon. Next, the inventors subcloned the tRNA sequences into the lentivirus vector without a promoter using MluI and SalI RE sites. Site directed mutagenesis was used to generate mutant Arg-TCT-4-1 by substituting T in position 34 to C (T34>C); thus changing the anticodon from TCT into CCT. Renilla luciferase reporter plasmid was generated by de novo gene synthesis (GeneWiz) modifying all the arginine codons (16 total) to AGA in the Renilla open reading frame. Codon modified Renilla was then subcloned into a psiCheck2 plasmid (Promega) using NheI and XhoI RE sites (psiCheck2-RLuc-AGA-sensor). Hmga2-WT and codon modified Hmga2-MUT (All AGA codons changed to CGC) were generated by de novo gene synthesis (GeneWiz). mCherry was inserted into the multiple cloning site of the bidirectional promoter vector pBi-CMV2 expressing a acGFP1 reporter (Clontech) using BamHI and NheI RE sites. Hmga2-WT and Hmga2-MUT open reading frames were then inserted into the C terminus of mCherry using NheI and HindIII RE sites generating mCherry-Hmga2-WT and mCherry-Hmga2-MUT respectively. Primer sequences can be found in Table S8 of Orellana, et al., supra.

### Recombinant Protein Purification

Recombinant wild-type and catalytic dead mutant Flag-METTL1 (L160A, D163A) proteins were co-expressed with wild-type 6×His-WDR4 and purified using Ni-NTA Agarose (QIAGEN). pETDuet-1 METTL1-Wt/WDR4 and METTL1-Mut/WDR4 were transformed into BL21 bacteria for induced expression of recombinant proteins. Bacteria were inoculated and cultured in LB medium at 37° C. Recombinant protein expression was induced (OD 0.4-0.6) using 0.5 mM IPTG at 20° C. overnight. Next, the bacteria were collected and lyzed by sonication, centrifuged at 15,000 rpm at 4° C. for 60 min. The cleared supernatant was collected and recombinant methyltransferase complexes were purified using Ni-NTA Agarose (QIAGEN) to capture 6×His-WDR4 following the manufacturer's instructions.

### In Vitro Methylation Assay

Full length wild-type tRNA-Val(TAC) or mutant (G46>C) RNA probes (200 pmol, Dharmacon) were incubated with recombinant METTL1-Wt/WDR4 or METTL1-Mut/WDR4 (500 ng) in the presence of H³—S-adenosylmethyonine (1 pM) for one hour at 37° C. in 1× reaction buffer (Tris pH 7.4 20 mM, DTT 1 mM, NaCl 50 mM, KCl 50 mM, MgCl₂ 1 mM, Glycerol 4%, RNAsin 0.2 U/pL). A no-protein treatment was used to measure background radiation. Next, RNA was purified using Oligo Clean and Concentrator™ kit (Zymo) following the manufacturer's instructions. Eluted RNA (20 pL) was then mixed with 5 mL of Ultima Gold™ scintillation buffer (Perkin Elmer) and radiation levels were measured using a Tri-Carb 2910 TR instrument (Perkin Elmer).

### Copy Number Analysis

Genomic DNA was isolated using Quick-DNA™ microprep kit (Zymo) following the manufacturer's instructions. METTL1 copy number alteration was evaluated using gene specific TaqMan Copy Number Assay (Thermo Fisher Scientific) according to the manufacturer's instructions. RNase P was used as a reference gene and T98G human GBM cell line was used as the calibrator sample (CCLE).

Cellular RNA Degradation Reactions (meCLICK)

MOLM13 cells were transduced with an Empty or a METTL1 gRNA as described above. On day 5 post-transduction the cells were suspended in methionine-free RPMI-1640 media (GIBCO) supplemented with 10% v/v FBS and 1% v/v penicillin/streptomycin/1-glutamine at a density of 1 000 000 cells mL-1. The cells were incubated for 30 min at 37° C. followed by addition of PropSeMet at a final concentration of 150 pM. Treated cells were incubated for a further 16 h at 37° C. Aqueous solutions of premixed CuSO4 and THPTA were added at final concentrations of 100 and 300 pM, respectively, followed by the click-degrader at 400 pM and NaAsc at 5 mM. Treated cells were incubated for 10 min at 37° C. and resuspended in complete RPMI-1640 medium. Afterward, the cells were again incubated at 37° C. and harvested after 5 h for RNA extraction. Total RNA was extracted from pelleted cells using micro-RNAeasy™ Kit (QIAGEN) according to the manufacturer's instructions. One microgram of total RNA was retrotranscribed using Super-Script™ Vilo Master Mix (Invitrogen) according the to manufacturer's instructions. Levels of specific RNAs were measured using fast mode of StepOnePlus™ Real-Time PCR System (Applied Biosystems) and Fast SYBR Green Master Mix (Applied Biosystems) according to the manufacturer's instructions. RNA levels were normalized to 18S subunit of the ribosome. Primer sequences are listed in Table S8 of Orellana, et al., supra.

### Quantitative RT-PCR

Total RNA was isolated from cancer cells using the RNeasy™ Mini (QIAGEN, 74104) or miRNeasy Kit (QIAGEN, 217004). For cDNA synthesis, total RNA was reverse-transcribed with the SuperScript VILO™ cDNA Synthesis kit (Life Technologies, 11754050). The levels of specific RNAs were measured using the ABI 7900 or StepOne™ real-time PCR machines and the Fast SybrGreen™ PCR master-mix (ThermoFisher, 4385612) according to the manufacturer's instructions. All samples, including the template controls, were assayed in triplicate. The relative number of target transcripts was normalized to 18S subunit of the ribosome or RPLP0. The relative quantification of target gene expression was performed with the standard curve or comparative cycle threshold (C_{T}) method. The primer sequences are listed in Table S8 of Orellana, et al., supra.

### Immunohistochemistry Staining

The brain glioblastoma cancer tumor array was purchased from Biomax (GL805e). The slide was baked for 60 minutes in an oven set to 60° C. Following deparaffinization and rehydration, antigen retrieval was performed using antigen unmasking reagent (citrate based) in a pressure cooker for 2.5 minutes and let to cool down for 30 minutes. Blocking was performed by incubating the slide in BloxAll™ (Vector Labs, SP-6000) for 10 minutes followed by incubation in 2.5% horse serum solution for 20 minutes. Then the slide was incubated with METTL1 antibody (Protein Group, 14994-1) at 1:200 or WDR4 antibody (Abcam, EPR11052) at 1:6000 for 60 minutes at room temperature. Primary antibody was detected using Impress Excell™ staining kit (Vector Labs, MP-7601). Slides were developed in DAB, then dehydrated and coverslipped. Each sample was scored by the percentage of positively stained cells and the staining intensity (intensity score: 0-3). Then the sample staining score was calculated by multiplying the percentage score and the intensity score.

### Virus Production and Generation of Stable Knockdown and Overexpression Cells

Generation of stable knockdown and overexpression cells via virus transduction was performed described previously (Lin et al., 2016). In brief, shRNA containing pLKO.1 vector was co-transfected with pLP1, pLP2, and VSVG into 293T cells. For overexpression, pBabe vectors containing the wild-type METTL1 (METTL1-Wt), METTL1 catalytic dead mutant (L160A-D163A, METTL1-Mut), and WDR4 were co-transfected with Gag-Pol and VSVG plasmids into 293T cells. Viruses were collected at 48 h and 72 h after transfection and then used to infect cells; 48 h after infection, puromycin (2.5 ug/mL) or G418 (400 ug/mL) was added to the culture medium to select the infected cells. MEF-WT cells overexpressing METTL1-Wt, METTL1-Mut, or empty vector were maintained in medium supplemented with puromycin (2.5 ug/mL) and G418 (400 μg/mL). LNZ308, LP6, LPS853, and 93T449 cells infected with shMETTL1, or shGFP were maintained in medium with puromycin (2.5 ug/ml). For CRISPR studies, viruses were prepared as follows: 293T cells were transfected with the appropriate lentiviral vector together with the packaging plasmids PAX2 and VSVg at a 1:1.5:0.5 ratio. Supernatant was harvested 48 and 72 h after transfection. 5×10⁵ cells and viral supernatant were mixed in 2 ml culture medium supplemented with 8 μg ml⁻¹ (human) or 4 μg ml⁻¹ (mouse) polybrene (Merck, H9268), followed by spinfection (60 min, 900 g, 32° C.) and further incubated overnight at 37° C. The medium was refreshed on the following day and the transduced cells were cultured further.

### Generation of Cas9-Expressing Cancer Cell Line

All Cas9-expressing cancer cell lines for screening were transduced with a virus produced from pKLV2-EF1aBsd2ACas9-W as previously reported (Tzelepis et al., 2016). Briefly, blasticidin selection was initiated 3 days after transduction at 10 μg ml⁻¹ for all cell lines. After stable cell lines were established, the transduced cells were single-cell sorted into 96-well plates (MoFlo XDP). Cas9 activity in individual subclones was tested using a lentiviral reporter pKLV2-U6gRNA(gGFP)-PGKBFP2AGFP-W. For CRISPRa assays, MOLM-13 cells were electroporated in Buffer R (Invitrogen) with 200 ng of plasmid encoding PiggyBac transposase together with 1 μg dCas9:SAM to facilitate stable integration as previously reported (Yang et al., 2019). Electroporation was performed using the Neon Transfection System (Thermo Fisher Scientific). Electroporation conditions used for MOLM-13 cells were based on manufacturer's instructions (1350V, 35 ms, 1 pulse). 2 days after electroporation, cells were then selected by 10 μg/ml Blasticidine (GIBCO, A1113903) for 10 days before further experiments performed. Post-selection, dCas9:SAM expressing MOLM-13 cells were expanded to 100×10⁶ cells for lentiviral transduction.

### Generation of Conditional Knock-Down Cells

B16F10 cells (3×10⁵) were infected as described above using PLKO-TETon-Puro lentiviral vectors expressing shRNAs against the coding sequence of mouse METTL1 or a were replated in fresh medium containing 1 μg ml⁻¹ of puromycin and kept in selection medium for 7 days. shRNA was induced by treatment with 500 ng ml⁻¹ doxycycline (Merck, D9891) for the indicated times. The shRNA sequences are listed in Table S8 of Orellana, et al., supra.

gRNA Competition Assays

gRNA competition assays were performed using single gRNA vectors as described previously (Tzelepis et al., 2016). For the validation of individual target genes, gRNAs were designed using software available on the world wide web at sanger.ac.uk/htgt/wge/. Viral supernatants were collected 48 h after transfection. All transfections and viral collections were performed in 24-well plates and transduction was performed as mentioned above. For gRNA/BFP competition assays, flow cytometry analysis was performed on 96-well plates using a LSRFortessa instrument (BD). Gating was performed on live cells using forward and side scatter, before measuring of BFP cells. The gRNA sequences are listed in Table S8 of Orellana, et al., supra.

### Cell Proliferation, Apoptosis, and Cell Cycle Analyses

In brief, for cell proliferation, 8×10⁴ cells were seeded in a 12-well plate on day 0. MEF-WT and LNZ308 Cells were trypsinized and counted on day 2 and day 4 to measure proliferation using a TC20 cell counter (Bio-Rad). B16F10 control or METTL1-KD cells (1×10⁵; 4 days after doxycycline induction) were seeded in 2 ml complete DMEM medium and counted 4 days after plating using the Countess II cell counter. For METTL1 overexpression experiments, MOLM-13 and THP-1 cells transduced with the indicated lentiviral cDNA vectors, then 1×10⁵ cells were seeded in 2 ml complete RPMI medium and counted 5 and 8 days after plating using the Countess II cell counter. For apoptosis and cell cycle analyses, 6×10⁶ cells were seeded in a 6-well plate and cells were collected 24 hours later. The numbers of apoptotic cells were quantified by flow cytometric assays using Annexin V-FITC Apoptosis Detection Kit (BioVision) according to the manufacturer's instructions. Cell cycle analyses were performed using flow cytometry after labeling cells with bromodeoxyuridine (BrdU) using the FITC BrdU Flow Kit (BD PharMingen, 559619) or the APC BrdU Flow Kit (BD Pharmigen, 51-9000019AK) following the manufacturer's instructions. Briefly, 1×10⁶ cells were incubated with 10 M at 37° C. with 5% CO₂ in air for 1 hour. After 1 hour of pulse, cells were washed three times to remove unincorporated BrdU, and fresh medium was added. For pulse-chase time course experiments cell were incubated for different amounts of time following BrdU labeling. Cells were fixed and DNA stained using 7-aminoactinomycin D (7-AAD). Cells were analyzed using the BDFortessa LSRII Cell Analyzer (BD Pharmigen). For in vivo assays using MOLM-13 cells, bone marrow cells were collected from mice transplanted with human AML cells 10 days before. Then human AML cells were enriched using human CD45 microbeads (Miltenyi, 130-118-780) and 1×10⁶ human cells were used for cell cycle analysis using the APC BrdU Flow Kit according to manufacturer's protocol (BD Pharmigen, 51-9000019AK). Data were analyzed by using LSRFortessa (BD) instruments. For in vivo assays using B16F10 cells, subcutaneous tumors were collected from mice transplanted with mouse melanoma cells 10 days before. Then 1×10⁶ mouse melanoma cells were used for cell cycle analysis using the APC BrdU Flow Kit according to manufacturer's protocol (BD Pharmigen, 51-9000019AK). Data were analyzed by using LSRFortessa (BD) instruments.

### Soft Agar Colony Formation Assays

Fifty thousand single live MEFs cells were mixed with 0.35% top-agar (SeaPlaque, Lonza) and were plated onto 0.7% base-agar (SeaPlaque, Lonza) in six-well plates. Twenty-eight days after plating the cells into soft agar, colony numbers were counted. The plates were imaged using a EVOS FL auto plate imager (Thermo Fisher Scientific) under continuous scan. Images were stitched and the colony numbers were counted using ImageJ.

### Isolation of Hematopoietic Progenitors

Flt3^{ITD/+} mice² were kindly provided by Gary Gilliland and crossed with Rosa26^{Cas9/+} mice. Freshly isolated bone marrow from 6- to 10-week-old female Rosa26^{Cas9/+}, NraS^{G12/+}, Flt3^{ITD/+}; Rosa26^{Cas9/+} or moribund Npm1^{flux-ct/+}; Flt3^{ITD/+}, Npm1^{flux-ct/+}; Nras^{G12D/+} mice were used. Bone marrow cells were exposed to erythrocyte lysis using BD PharmLyse (BD Bioscience, 555899), followed by magnetic bead selection of Lin-cells using the Lineage Cell Depletion Kit (Miltenyi Biotec, 130-090-858) according to the manufacturer's instructions. Lin were cultured in X-VIVO™ 20 (04-448Q, Lonza) supplemented with 5% BIT serum (09500, Stem Cell Technologies) 10 ng ml⁻¹ IL3 (Peprotech, 213-13), 10 ng ml⁻¹ IL6 (216-16, Peprotech) and 50 ng ml⁻¹ of SCF (Peprotech, 250-03). Retrovirus constructs pMSCV-MLL-AF9-IRES-YFP and pMSCV-MLL-ENL-IRES-Neo were used with package plasmid psi-Eco to produce retrovirus. 293T cells were cultured and prepared for transduction in 10 cm plates as described above. For virus production, 5 μg of the above plasmids and 5 μg psi-Eco packaging vector were transfected drop wise into the 293T cells using 47.5 μL TransIT LT1 (Mirus, MIR 2304) and 600 μL Opti-MEM (Invitrogen, 31985062). The resulting viral supernatant was harvested as previously described. Transduction of primary mouse cells was performed in 6-well plates as mentioned above. After transduction, transduced cells were sorted for YFP (for MLL-AF9) or selected with neomycin (for MLL-ENL). For re-plating assays using gRNA or cDNA constructs, 5,000 lineage negative cells and primary murine AML cells were plated in three wells of 6-well-plate of M3434 methylcellulose (Stem Cell Technologies, 03434) after selection with 1.0 μg ml⁻¹ puromycin for 3 to 5 days starting from day 2 post transduction. The colonies were counted 7 days later and further 5,000 cells re-seeded and re-counted after a week until the 3rd re-plating.

### Adult Primary Leukemia and Cord Blood Sample Analysis

5×10⁵ human AML patient and cord-blood-derived CD34+ cells were pelleted and resuspended in whole cell lysis buffer (50 mM Tris-HCl pH=8, 450 mM NaCl, 0.1% NP-40, 1 mM EDTA), supplemented with 1 mM DTT, protease inhibitors (Sigma, S8820), and phosphatase inhibitors (Sigma, P5726-1ML). Protein concentrations were assessed by Bradford assay (Bio-Rad, 5000006) and an equal amount of protein was loaded per track. Prior to loading, the samples were supplemented with SDS-PAGE sample buffer and DTT was added to each sample. 10 μg of protein was separated on SDS-PAGE gels, and blotted onto polyvinylidene difluoride membranes (Millipore, IPVH00010). Cord-blood-derived CD34+ cells were tested for colony-forming efficiency in StemMACS HSC-CFU semi-solid medium (Miltenyi, 130-091-280) in the presence of the indicated shRNA vector for either scramble or METTL1. Colonies were counted by microscopy 12-14 days (CD34+ cells) after plating. All human primary and cord blood samples were obtained with informed consent under local ethical approval (REC 07-MRE05-44).

### Competitive Bone Marrow Transplantation

Freshly isolated bone marrow from 6- to 10-week-old female Rosa26Cas9/+ mice were used. Bone marrow cells were exposed to erythrocyte lysis using BD PharmLyse (BD Bioscience, 555899), followed by magnetic bead selection of Lin-cells using the Lineage Cell Depletion Kit (Miltenyi Biotec, 130-090-858) according to the manufacturer's instructions. Lin-cells were then cultured in X-VIVO™ 20 (04-448Q, Lonza) supplemented with 5% BIT serum (09500, Stem Cell Technologies) 10 ng ml⁻¹ IL3 (Peprotech, 213-13), 10 ng ml⁻¹ IL6 (216-16, Peprotech) and 50 ng ml⁻¹ of SCF (Peprotech, 250-03) overnight. Lin-cells were then transduced with a blank (no vector), an Empty-BFP or Mettl1-BFP gRNA and cultured for another 48 hours. On day 4 post bone marrow extraction, transduced Lin-cells with the BFP vectors were purified using cell sorting and mixed equally (50-50) with the Lin-cells from the blank cohort. Of those 2 individual pooled cohorts, 1×10⁶ cells were then transplanted to lethally irradiated (12 Gy) female C57BL/6J mice, with an addition of extra 5×10⁵ whole BM cells for additional support (final 1.5×10⁶ cells). The competitive chimerism was then monitored over the following 16 weeks by flow cytometry of the peripheral blood using a LSRFortessa instrument (BD). For evaluation of the METTL1 levels in both cohorts, bone marrow cells were isolated from mice 8 weeks post-transplantation and protein from 5×10⁶ cells extracted as described above.

High Performance Liquid Chromatography with Tandem Mass Spectrometry (HPLC-MS/MS) Analysis of RNA

250 ng to 500 ng RNA was digested with 0.5 U P1 nuclease at 37° C. for 2 hours and dephosphorylated with 1U rSAP at 37° C. for 1 hour. Then 100 μL samples were filtered with Millex-GV 0.22u filters. The RNA samples were not de-capped, hence the m⁷G measurements reflect internal m⁷G modification and do not include the m⁷G cap. 5 to 10 μL from each sample was injected into Agilent 6470 Triple Quad LC/MS instrument. The samples were run in mobile phase buffer A (water with 0.1% Formic Acid) and 2 to 20% gradient of buffer B (Methanol with 0.1% Formic Acid). MRM transitions were measured for guanosine (284.1 to 152.1), 7-methylguanosine (m⁷G) (298.1 to 166.1). For LC/MS-MS data collection and analysis the following software was used: Agilent Mass Hunter LC/MS Data Acquisition Version B.08.00 and Quantitative Analysis Version B.07.01.

Isolation of tRNAs for HPLC-MS/MS Analysis

To isolate Arg-TCT tRNA, 300 pmol of synthetic DNA oligos complementary to Arg-TCT isodecoders 1,2,3,5 (probe 1) or Arg-TCT isodecoder 4 (probe 2) were incubated with 300 μg of total RNA in 50 μL of annealing buffer (10 mM Tris pH8.0, 1 mM EDTA, 50 mM NaCl) and incubated at 95° C. for 5 minutes and slowly cooled to room temperature (25° C.) over 3.5 hours to allow hybridization to occur. The hybridized products were incubated with streptavidin C1 beads (Invitrogen) in IP buffer (1M NaCl, 5 mM Tris pH7.5, 0.5 mM EDTA) at 4° C. for 1 hour. After extensive washing, beads were heated at 95° C. for 5 min and supernatant (containing tRNAs) was collected. Next, RNA in supernatant was size-selected (<200nt) and concentrated using Zymo RNA clean and purification kit (Zymo). Biotinylated oligo sequences are listed in Table S8 of Orellana, et al., supra.

m⁷G tRNA mcRIP

m⁷G tRNA mcRIP was performed as previously described (Lin et al., 2018). Briefly, small RNAs (<200nt) were purified using the mir-Vana miRNA Isolation Kit (Thermo Fisher Scientific). Then anti-m⁷G mcRIP was performed on the small RNA by incubating 10 ug small RNAs with 10 ug anti-m⁷G antibody (MBL International, #RN017M) or normal rabbit IgG control (Cell Signaling, #2229) for 2 hr at 4 C. Next, 50 ul pre-washed Protein A/G Magnetic Beads (Thermo Fisher Scientific, #88802) were added to purify the m⁷G modified small RNAs and incubated for 2 hours at 4 C. The beads were washed extensively and the bead bound RNAs were dissociated by boiling the beads in 1× urea loading buffer (Invitrogen) for 5 minutes.

### m⁷G TRAC-Seq

m7G TRAC-Seq, mcRIP was performed as previously described (Lin et al., 2018, 2019). First, isolated small RNAs were first treated with recombinant wild-type and D135S AlkB proteins to remove the dominant methylations on RNAs as previously described. Briefly, 10 ug small RNAs were treated with 80 pmol wt AlkB and 160 pmol D135S AlkB mutant for 2 hours in a 100 ul demethylation reaction [300 mM KCl, 2 mM MgCl₂, 50 mM of (NH₄)₂Fe(SO₄)2·6H2O, 300 mM 2-ketoglutarate (2-KG), 2 mM L-ascorbic acid, 50 mg/mL BSA, 50 mM MES buffer (pH 5.0)] at room temperature. After incubation, the reaction was quenched with at final concentration of 5 mM EDTA and the RNAs were purified by phenol-chloroform extraction followed by ethanol precipitation. Alkb-treated RNAs (2.5 ug) were then treated with 0.1M NaBH₄ for 30 min on ice at dark in the presence of 1 mM free m⁷GTP as methylation carrier. Then the RNAs were precipitated with sodium acetate (300 mM final concentration, PH5.2) and 2.5 volumes of cold ethanol at −20C overnight. After precipitation, the NaBH₄-treated RNAs were subsequently treated with aniline-acetate solution (H2O:glacial acetate acid:aniline, 7:3:1) at room temperature at dark for 2 hours to induce the site-specific cleavage. After cleavage, the RNA samples were purified by ethanol precipitation and used for cDNA library construction using NEBNext Small RNA Library Prep Set (New England Biolabs) followed by sequencing with Illumina Nextseq™ 500.

m⁷G Site Calling from TRAC-Seg

Adaptor sequences were trimmed and low-quality sequences (Q20) were discarded using trim_galore (available on the world wide web at bioinformatics. babraham.ac.uk/projects/trim_galore). For tRNA chemical sequencing data, clean reads were mapped to the mature tRNA sequences downloaded from GtRNAdb using Bowtie™ with a maximum of two mismatches allowed (Langmead et al., 2009). The alignments were then processed to record the read depth of each site on tRNAs using Bedtools (Quinlan, 2014). Based on tRNA mapping bam files and read depth information, cleavage score and cleavage score ratio between input and chemically-treated sample using the program cleavage_score.R (available on the world wide web at github.com/rnabioinfor/TRAC-Seq)(Lin et al., 2018, 2019). The positions with a cleavage score>3 and the cleavage ratio>0.2 in both samples were considered as the candidate m⁷G sites. Based on 21 bp sequences around m⁷G sites, the enriched motifs were analyzed by MEME with a maximum 7 bp width. To conduct tRNA expression analysis for Input samples, the inventors used the programs ARM-Seq data analysis pipeline (Cozen et al., 2015). Differential expression analysis can be then performed based on the output count file by using DESeq (available on the world wide web at bioconductor.org/packages/release/bioc/html/DESeq2.html).

### Northern Blot, Northwestern Blot, and Western Blot

For Northern blotting of tRNAs or U6 snoRNA, 2 ug total RNA samples were mixed with 2× TBE loading buffer (Bio-Rad) and incubated at 95° C. for 5 min. The samples were then loaded into 15% TBE-UREA (Bio-Rad) gels to separate the RNAs by molecular weight. Next, the RNAs were transferred onto a positive charged nylon membrane and crosslinked with UV. For Northern blots, the membrane was blotted with radioactive labeled probes against tRNAs or U6 snRNA. Acid urea PAGE (10%) was used to evaluate amino-acylation levels in presence of 10 mM CuSO₄. The probe sequences are listed in Table S8 of Orellana, et al., supra. Bolted membranes were then exposed to autoradiography films. For Northwestern blot, membranes were immunoblotted with mouse monoclonal anti 7-methylguanosine (m⁷G) (MBL International, RN017M). For western blotting, cells were transduced with lentiviral gRNA, shRNA or cDNA vectors and selected with 1.0 mg ml⁻¹ puromycin for 3 days starting from day 2 post transduction. The transduced cells were further cultured for 5 days before lysis. Cell pellets resuspended in whole cell lysis buffer (50 mM Tris-HCl pH=8, 450 mM NaCl, 0.1% NP-40, 1 mM EDTA), supplemented with 1 mM DTT, protease inhibitors (S8820, Sigma), and phosphatase inhibitors (P5726-1ML, Sigma). Protein concentrations were assessed by Bradford assay (5000006, Bio-Rad) and an equal amount of protein was loaded per track. Prior to loading, the samples were supplemented with SDS-PAGE sample buffer and DTT was added to each sample. 10-40 mg of protein was separated on SDS-PAGE gels, and blotted onto polyvinylidene difluoride membranes (IPVH00010, Millipore). Membranes were then immunoblotted with the following antibodies: B-Actin (Abcam, ab8229 or ab8227), METTL1 (Protein Group, 14994-1 or Abcam, ab157097), WDR4 (Abcam, EPR11052), HMGA2 (Abcam, ab97276; or Cell Signaling, 8179), KDM1/LSD1 antibody (Abcam, ab17721), ASH2L (Proteintech, 12331-1-AP), SETDB1 (Proteintech, 11231-1-AP), CDK4 (Proteintech, 11026-1-AP).

### RNA-Seq Analysis of Patient Samples

RNA-seq data of healthy and AML patients from the Leucegene dataset were downloaded from four individual studies (Lavallée et al., 2016; Macrae et al., 2013; Pabst et al., 2016; Simon et al., 2012). Mapping to the human genome assembly GRCh38 and read counts were performed by STAR v2.7 (Dobin et al., 2013). Reads were normalized to effective exon lengths and then to the upper quartile of each sample. Log10 values of gene expression were shown as box-and-whiskers and p values were computed using two-tailed t tests.

### Ribosome Footprinting (Ribo-Seq)

Immortalized MEFs stably transduced with either empty vector (EV), wild-type METTL1 and WDR4, or mutant METTL1 and WDR4 were grown to 80%-90% confluence in DMEM supplemented with 10% FBS in 15-cm dishes. Ribosome footprinting was performed according to TruSeq® Ribo Profile system (Illumina) with modifications. Briefly, cells were treated with 0.1 mg/mL cycloheximide (CHX) for 1 minute to inhibit translation elongation and washed with ice-cold PBS containing 0.1 mg/mL CHX. Next, cells were lyzed in 800 ml 1× Mammalian Polysome Buffer (Illumina) supplemented with 1% Triton X-100, 1 mM DTT, 10 units DNase I, 0.1 mg/mL CHX, and 0.1% NP-40. Lysates were cleared by centrifugation at 12,000 μg for 10 min at 4° C., and supernatants were flash-frozen in liquid nitrogen and stored at −80° C. until processing. RNA concentration of the lysates was measured according to their absorbance at A₂₆₀ and an equivalent of 12×A₂₆₀/ml was treated with 5 U/A₂₆₀ TruSeq™ Ribo Profile RNase Nuclease (Illumina) for 45 minutes at room temperature. RNase activity was inhibited by adding 15 μl SUPERase to the mixture. Ribosome protected fragments (RPFs) were isolated using MicroSpin S-400 columns (GE Healthcare). RPF RNA samples (5 g) were subjected to ribosomal RNA depletion using RiboMinus™ Eukaryote Kit v2 (Thermo Fisher Scientific). Ribo-depleted RNA samples were separated on 15% polyacrylamide/TBE/Urea gels (Thermo Fisher Scientific) and the RNA fragments corresponding to ˜25-35 nt were excised. RNA was gel-extracted and precipitated overnight at 4° C. using 0.5 M ammonium acetate. In parallel, total RNA input samples were isolated, and fragmented at 94° C. for 25 minutes. Input total RNA and RPFs were subjected to end repair by TruSeq™ Ribo Profile PNK (Illumina), cleaned using RNA Clean & Concentrator-5 kit (ZYMO Research) and ligated to 2.5 pM Universal miRNA Cloning Linker (NEB) by using 100 units T4 RNA Ligase 2, truncated KQ (NEB) for 3 hours at 22° C. After ligation, RNA samples from both total RNA and RPFs were reverse transcribed using SuperScript™ III Reverse Transcriptase (Thermo Fisher Scientific) and 0.25 pM RT primer (IDT). cDNA samples were then gel-extracted on 10% polyacrylamide/TBE/Urea gels (Thermo Fisher Scientific) and circularized by CircLigase ssDNA Ligase (Lucigen) for 2 hours at 60° C. cDNA libraries for total RNA and RPFs were amplified for 9 and 12 PCR cycles, respectively, using Phusion High-Fidelity PCR Master Mix (NEB), Illumina index primers and 10 M forward primer (IDT). Amplified libraries were cleaned using AMPure™ XP Beads (Beckman Coulter), followed by gel-extraction on 8% native TBE gels (Thermo Fisher Scientific). Libraries were sequenced with Illumina NextSeq™ 500. The primer sequences are listed in Table S8 of Orellana, et al., supra.

### Ribo-Seq Data Analysis

The sequences of input and Ribo-seq samples were first processed to get the clean reads by trimming the adapters and filtering the low-quality sequences. Then, for Ribo-seq input data, the clean reads were aligned to reference genome sequences using STAR (Dobin et al., 2013). The resulted BAM mapping files were used as inputs of HTSeq (Anders et al., 2015) to calculate the read counts for each gene from GENCODE gene mode. For the cleaned Ribo-seq data, the clean ribosome-protected fragments (RPFs) were first collapsed into FASTA format by fg2collapedFa. RiboToolkit (available on the internet atrnabioinfor.tch.harvard.edu/RiboToolkit/) was used to perform codon occupancy analysis and translation efficiency analysis by uploading the collapsed RPF tags and gene read counts from input samples (Liu et al., 2020a). In brief, rRNA and tRNA sequences were filtered from RPF containing files by alignment to rRNA sequences (Ensembl non-coding, release 91) (Zerbino et al., 2018) and tRNA sequences from GtRNAdb databases (Chan and Lowe, 2016). The resulting ribosome-protected fragments (RPFs) were aligned to the mouse reference genome (mm10) using STAR [10.1093/bioinformatics/bts635] and only unique mapped reads were kept. The genome unique mapping reads were then mapped to transcript sequences using Bowtie™ with a maximum of one mismatch allowed (Langmead et al., 2009). and all the transcript mappings were kept. CONCUR tool (https://github.com/susbo/concur) was used for calculating codon usage based on the reference genome mapping based on STAR (Frye and Bornelov, 2021). The translation efficiency (TE) was calculated by dividing RPF abundance on CDS by its mRNA abundance of input sample. A threshold of two-fold change and FDR<0.05 was used to define the differential translation genes (Zinshteyn and Gilbert, 2013). PausePred (available on the world wide web at pausepred.ucc.ie/), was used to infer ribosome pauses from Ribo-seq data. Peaks of ribosome footprint density are scored based on their magnitude relative to the background density within the surrounding area (Kumari et al., 2018).

### Proteomic Analysis by Stable Isotope Labeling Using Amino Acids in Cell Culture (SILAC)

LNZ308 human glioblastoma cells or MEF-WT immortalized cells were grown in media supplemented with isotopic-labeled ¹³C₆¹⁵N₂ 1-lysine and ¹³C₆¹⁵N₄ 1-arginine (heavy) or normal amino acids (light) for 15 to 21 days until a labeling efficiency >95% was achieved following the instructions of the SILAC Protein Quantitation Kit (Trypsin)—DMEM (A33972, Thermo Scientific). Light SILAC-labeled shGFP LNZ308 and Empty-vector MEF-WT cells as well as heavy-labeled shMETTL1 LNZ308, METTL1/WDR4 and Arg-TCT-4-1 overexpressing MEF-WT cells were lysed in 1× passive cell lysis buffer (Promega) supplemented with cOmplete protease inhibitor (11873580001, Roche). Protein lysates were cleared by centrifugation at 14,000×g for 5 minutes at 4° C. Equal amounts of heavy and light protein (1:1) amounts were mixed with 2× reducing sample buffer and 100 μg of clarified sample was separated by SDS-PAGE (4%-20%). Three technical replicates were performed for each sample. Gels were stained using Novex Colloidal blue staining (Invitrogen) and each lane was cut into 12 slices of equal size. Excised gel bands were cut into approximately 1 mm³ pieces. The samples were reduced with 1 mM DTT for 30 minutes at 60° C. and then alkylated with 5 mM iodoacetamide for 15 minutes in the dark at room temperature. Gel pieces were then subjected to a modified in-gel trypsin digestion procedure (Shevchenko et al., 1996). Gel pieces were washed and dehydrated with acetonitrile for 10 min. followed by removal of acetonitrile. Pieces were then completely dried in a speed-vac. Rehydration of the gel pieces was with 50 mM ammonium bicarbonate solution containing 12.5 ng/pl modified sequencing-grade trypsin (Promega, Madison, WI) at 4° C. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (A1 hr). The samples were then stored at 4° C. until analysis. On the day of analysis, the samples were reconstituted in 5-10 μl of HPLC solvent A (2.5% acetonitrile, 0.1% formic acid). A nano-scale reverse-phase HPLC capillary column was created by packing 2.6 pm C18 spherical silica beads into a fused silica capillary (100 pm inner diameter×˜30 cm length) with a flame-drawn tip (Peng and Gygi J). After equilibrating the column each sample was loaded via a Famos auto sampler (LC Packings, San Francisco CA) onto the column. A gradient was formed and peptides were eluted with increasing concentrations of solvent B (97.5% aceto-nitrile, 0.1% formic acid). As each peptide was eluted they were subjected to electrospray ionization and then they entered into an LTQ Orbitrap Velos Pro ion-trap mass spectrometer (Thermo Fisher Scientific, San Jose, CA). Eluting peptides were detected, isolated, and fragmented to produce a tandem mass spectrum of specific fragment ions for each peptide. Peptide sequences (and hence protein identity) were determined by matching protein or translated nucleotide databases with the acquired fragmentation pattern by the software program, Sequest (ThermoFinnigan, San Jose, CA) (Eng. et al., 1994 J. Am. Soc. Mass. Spectrom). The differential modification of 8.0142 and 10.0083 mass units for lysine and arginine, respectively were included in the database searches to find SILAC labeled peptides. All databases include a reversed version of all the sequences and the data was filtered to between a one percent or lower peptide false discovery rate. SILAC protein ratios (H/L) were determined as the average of all peptide ratios assigned to a protein between heavy and light samples. Differential protein expression was determined using a moderated t test, testing for the null-hypothesis being no change in H/L ratio. Multiple tests were corrected using false discovery rate (<1%). Codon usage was estimated by dividing the number of specific codons in the coding sequence (CDS, observed) of an mRNA divided by the genome average number of each codon (expected) followed by a normalization to the CDS length (Supek and Vlahovicek, 2005).

### TCGA Data Analysis

RNA-Seq expression data and small RNA-Seq data for 33 TCGA tumor types were downloaded from the Genomic Data Commons Data Portal (GDC) of TCGA (available on the world-wide web at cancer.gov/about-nci/organization/ceg/research/structural-genomics/tega). The gene expression matrix was then constructed by merging the TPM (Transcripts Per Million) values of all RNA-seq samples. The tumor types with normal tissues were used to draw the gene expression boxplots and the statistic differences are then calculated using Wilcoxon rank-sum test (with asterisks indicating statistical significance). The expression correlations between METTL1 and WDR4 among TCGA tumors were conducted by using Pearson correlation coefficient. The tRNA expressions from TCGA small RNA-seq data were analyzed using ARM-Seq data analysis pipeline (Cozen et al., 2015). We used data generated by the Clinical Proteomic Tumor Analysis Consortium (NCI/NIH) to analyze METTL1 protein levels versus RNA transcripts.

### Luciferase Reporter Assay

Dual luciferase assays were performed according to the manufacturer's protocol (Promega). RLuc activity was normalized to the Renilla luciferase (FLuc) activity and the ratio was normalized to protein concentration. The normalized RLuc activity (translation efficiency) in the presence of empty vector was set to 1.

### Fluorescent Reporter Assay

In brief, 8×10⁵ MEF-WT cells overexpressing METT1/WDR4, Arg.TCT-4-1 or empty vector control were seeded in a 60 mm plate on day 0. On day 1, cells were transfected with 2 μg of mCherry-Hmga2-WT or mCherry-Hmga2-MUT using Lipofectamine 2000 (Life Technologies) and 48 hours later cells were trypsinized and resuspended in fresh medium to a density of 1×10⁶ cells/mL. Cells were analyzed using the BDFortessa LSRII Cell Analyzer (BD Pharmigen) and acGFP1 and mCherry expression was monitored. Data were analyzed using FlowJo V10.7 (Beckton Dickinson and Co.) and presented as a ratio between mCherry and acGFP1 intensity levels.

### Animal Studies

Research involving animals complied with protocols (BIDMC 102-2014) approved by the Beth Israel Deaconess Medical Center Institutional Animal Care and Use Committee. 4-6 weeks old female NU/J (Nude) immunodeficient mice (Jackson Laboratory #002019) were used for subcutaneous injections. To study the oncogenic transformation ability of METTL1/WDR4, MEF-WT cells (5×10⁶ cells) overexpressing METTL1-Wt/WDR4, catalytic dead mutant METTL1-L160A, D163A/WDR4 (METTL1-Mut/WDR4) or empty vector (EV) were transplanted into nude mice. To study the role of METTL1 in tumor formation, human LNZ308 glioblastoma cells or human LP6 liposarcoma cells (5×10⁵ cells) with stable METTL1 knockdown (shMETTL1) or negative control (shGFP) were transplanted. The indicated number of cells were mixed with serum-free medium and growth factor reduced Matrigel (Corning #354230) (1:1) and injected into the right flank of nude mice. Five or six mice were used for each group. Subcutaneous tumor formation was monitored by calipers twice a week. The tumor volume was calculated using the formula 1/2(length×width²). The recipient mice were monitored and euthanized when the tumors reached 1 cm in diameter. At end-point tumors were collected and weighted. For in vivo experiments with whole-body bioluminescent imaging the following protocol was used. MOLM-13 cells expressing Cas9 and B16F10 cells were first transduced with a firefly luciferase-expressing plasmid (System Biosciences, LL205PA-1). After propagation, the cells were transduced with the indicated lentiviral gRNA or doxycycline-inducible shRNA or cDNA vectors and selected with puromycin from day 2 to day 5. At day 5 post transduction, the cells were suspended in fresh medium without puromycin. At day 6, 5×10⁵ cells were transplanted into female 6- to 10-week-old NSGS mice or Rag2^{-/-} IL2RG^{-/-} mice by tail-vein (MOLM-13) or subcutaneous (B16F10) injections. For the in vivo B16F10 experiments, daily treatment of 2 mg/kg of doxycycline was administered via intraperitoneal injection (IP) on day 5 post-transplantation, for 10 consecutive days. Doxycycline (Sigma, D9891) were dissolved in 20% (w/v) 2-hydroxyproply beta-cyclodextrin vehicle (Sigma, H107). At day 5 post-transplant, the tumor burdens of the animals were detected using IVIS Lumina II (Caliper) with Living Image version 4.3.1 software (PerkinElmer). Briefly, 100 μL of 30 mg/ml D-luciferin (BioVision, 7903-1G) was injected into the animals intraperitoneally. Ten min after injection, the animals were maintained in general anesthesia by isoflurane and put into the IVIS chamber for imaging. The detected tumor burdens were measured and quantified by the same software. Diseased mice were assessed blindly by qualified animal technicians from the Sanger mouse facility. All animal studies were carried out in accordance with the Animals (Scientific Procedures) Act 1986, UK and approved by the Ethics Committee at the Sanger Institute. Randomization and blinding were not applied.

### Quantification and Statistical Analysis

Quantification and statistical analysis methods were described in individual method sections and Figure legends. Center is represented by mean and dispersion is represented by standard deviation. The n is reported in each figure legend. Paired or one-sided Student's t tests, Fisher's exact test or Wilcoxon's signed rank test were used for two group comparisons. One or two-way ANOVA were used for multigroup comparison. Bonferroni or Tukey post hoc p value corrections were performed for multigroup com-parisons. Pearson or Spearman tests were performed for correlation analysis. Randomization and blinding were not applied. The ROUT method was used to identify outliers (Q=1%). Data was considered significant if p values<0.05.

### Example 3

### Results and Conclusions

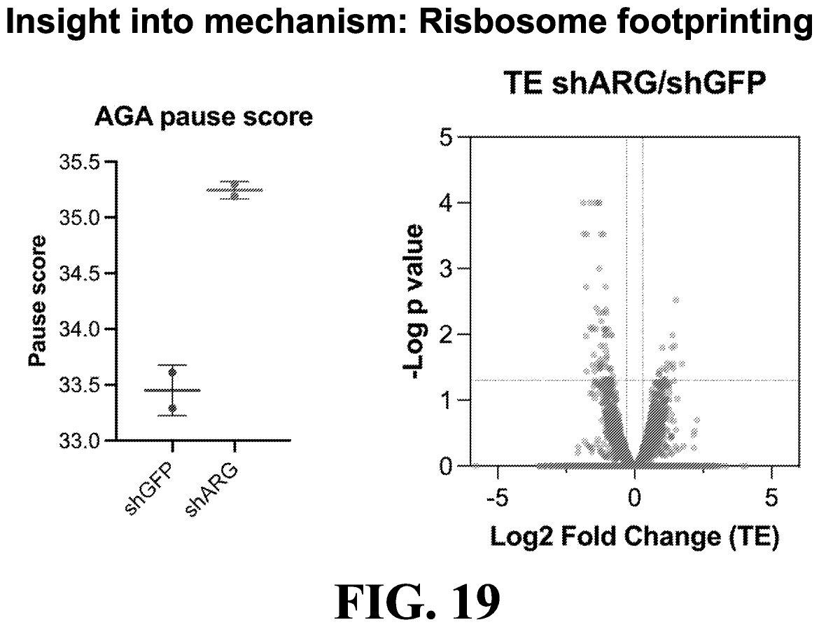

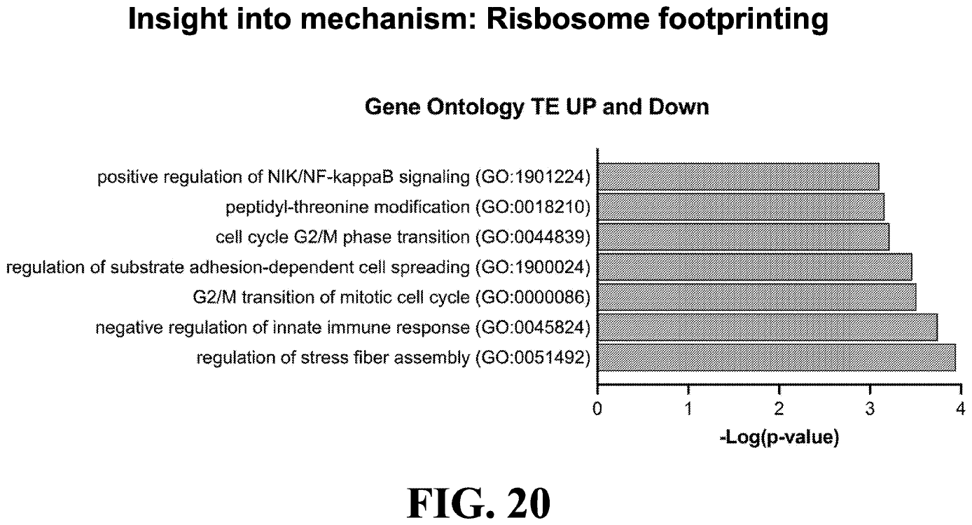

Inhibition of Arg-TCT-41 causes translation defects (FIGS. 19-20). To assess whether the reduction in oncogenic hallmarks after inhibition of Arg-TCT-4-1 was caused to defects in translation., ribosome footprinting (Ribo-Seq) was used to evaluate translation efficiency in cells treated with shARG compared to control cells shGFP. The results indicate that inhibition of Arg-TCT-4-1 causes an increase in AGA pauses in shARG-treated cells compared to the shGFP control. Moreover, a widespread reduction of translation efficiencies was observed in multiple genes. After performing gene ontology analyses (FIG. 19) the genes with altered translation efficiencies were found to be enriched with genes involved in cell cycle regulation. Altogether, these data support the idea that specific inhibition of Arg-TCT-4-1 can lead to defects in the translation of genes enriched in AGA codons that are involved in cell division.

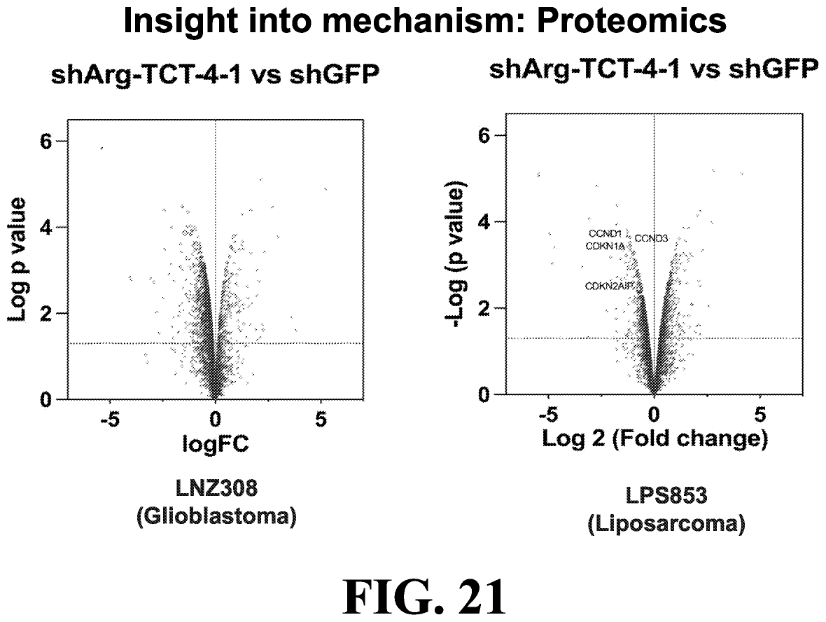

Inhibition of Arg-TCT-4-1 causes changes in the proteome (FIGS. 20-21). To assess whether the defects in translation efficiencies observed with Ribo-Seq, SILAC proteomics was next performed comparing shARG-treated cells versus shGFP control cells. The LNZ308 model of glioblastoma and LPS853 model of liposarcoma was used. The results indicate that inhibition of Arg-TCT-4-1 causes a widespread dysregulation of the proteome in shARG-treated cells compared to the shGFP control, including the downregulation of proteins involved in cell cycle control. Furthermore, the changes in protein abundance were compared in LPS853 cells treated with an antisense oligonucleotide targeting Arg-TCT-4-1 (ASO-1) compared to a scramble ASO control (FIG. 20). Pearson correlation analyses show that the changes in protein abundance after Arg-TCT-4-1 inhibition using shARG or ASO-1 are significant and positively correlated between the two datasets. This is highlighted by a large overlap in the set of downregulated proteins which are enriched in genes involved in response to protein misfolding (FIG. 21). Taken together, these data indicate that inhibition of ARG-TCT-4-1 in cancer cells leads to a widespread dysregulation of protein synthesis and activation of stress responses to deal with protein misfolding.

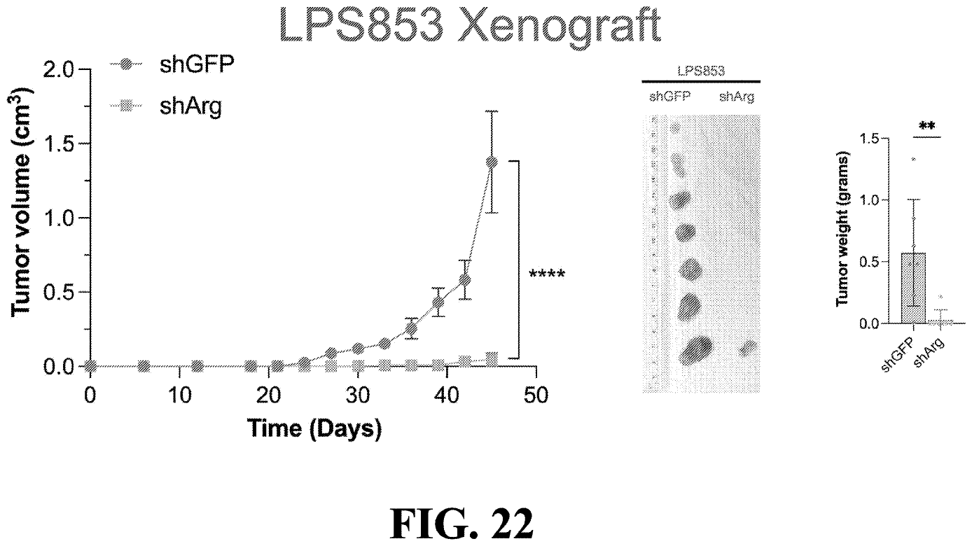

Inhibition of Arg-TCT-4-1 inhibits liposarcoma tumor formation in vivo (FIG. 22). Here, xenograft experiments were performed using liposarcoma LPS853 cells treated with shARG and shGFP control to test whether inhibition of Arg-TCT-4-1 can suppress tumor formation in vivo. These data are very similar to what we observed with the LNZ308 GBM model in slide #8. The data support the hypothesis that inhibition of ARG-TCT-4-1 could lead to tumor formation defects in cancers with high levels of expression of ARG-TCT-4-1.

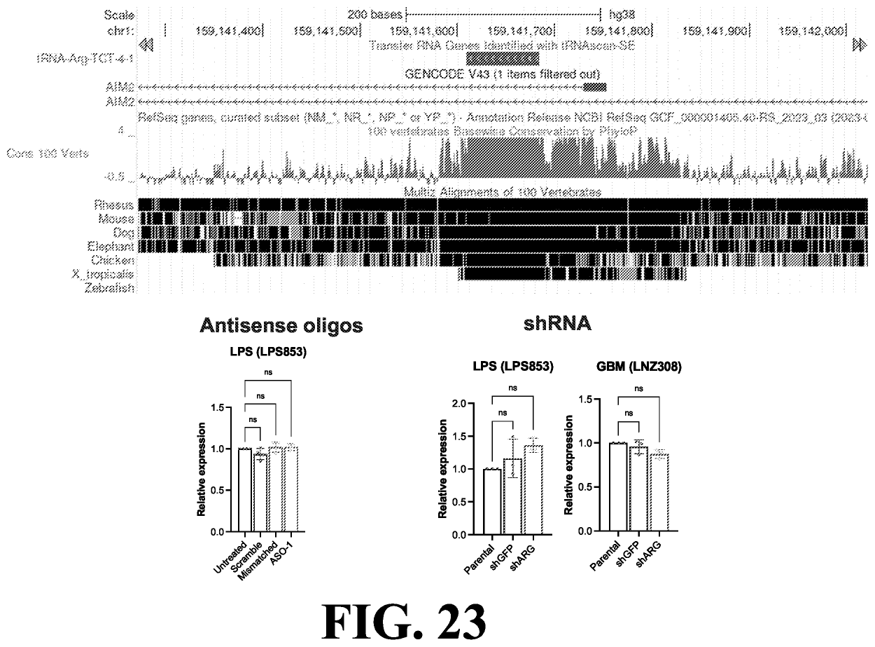

Inhibition of Arg-TCT-4-1 does not affect AIM %2 expression (FIG. 23). The gene for Arg-TCT-4-1 is harbored within an intronic region of a protein-coding gene called AIM2. It was tested if shRNA or ASO treatment can affect the expression of the host gene. Since the tRNA gene is located in an intron, the treatment was not expected to affect the expression of AIM2 mRNA. The data show that both shRNA or ASO treatment does not interfere with AIM2 mRNA expression and further illustrates the specificity of Arg-TCT-4-1 targeting.

### Methods

### Quantitative RT-PCR

Total RNA was isolated from cancer cells using Trizol. For cDNA synthesis, total RNA was reverse-transcribed with the SuperScript III cDNA Synthesis kit (Life Technologies). The levels of specific RNAs were measured using a StepOne real-time PCR machines and the Fast SybrGreen PCR mastermix (ThermoFisher, 4385612) according to the manufacturer's instructions. All samples, including the template controls, were assayed in triplicate. The relative number of target transcripts was normalized to B-Actin. The relative quantification of target gene expression was performed with the comparative cycle threshold (Cr) method.

### Ribosome Footprinting (Ribo-Seq)

Cancer cells were grown to 80%-90% confluence in DMEM supplemented with 10% FBS in 15-cm dishes. Ribosome footprinting was performed according to TruSeq® Ribo Profile system (Illumina) with modifications. Briefly, cells were treated with 0.1 mg/mL cycloheximide (CHX) for 1 minute to inhibit translation elongation and washed with ice-cold PBS containing 0.1 mg/mL CHX. Next, cells were lyzed in 800 μl 1× Mammalian Polysome Buffer (Illumina) supplemented with 1% Triton X-100, 1 mM DTT, 10 units DNase I, 0.1 mg/mL CHX, and 0.1% NP-40. Lysates were cleared by centrifugation at 12,000 μg for 10 min at 4° C., and supernatants were flash-frozen in liquid nitrogen and stored at −80° C. until processing. RNA concentration of the lysates was measured according to their absorbance at A₂₆₀ and an equivalent of 12×A₂₆₀/ml was treated with 5 U/A₂₆₀ TruSeq Ribo Profile RNase Nuclease (Illumina) for 45 minutes at room temperature. RNase activity was inhibited by adding 15 μl SUPERase to the mixture. Ribosome protected fragments (RPFs) were isolated using MicroSpin S-400 columns (GE Healthcare). RPF RNA samples (5 g) were subjected to ribosomal RNA depletion using RiboMinus Eukaryote Kit v2 (Thermo Fisher Scientific). Ribo-depleted RNA samples were separated on 15% polyacrylamide/TBE/Urea gels (Thermo Fisher Scientific) and the RNA fragments corresponding to ˜25-35 nt were excised. RNA was gel-extracted and precipitated overnight at 4° C. using 0.5 M ammonium acetate. In parallel, total RNA input samples were isolated, and fragmented at 94° C. for 25 minutes. Input total RNA and RPFs were subjected to end repair by TruSeq Ribo Profile PNK (Illumina), cleaned using RNA Clean & Concentrator-5 kit (ZYMO Research) and ligated to 2.5 M Universal miRNA Cloning Linker (NEB) by using 100 units T4 RNA Ligase 2, truncated KQ (NEB) for 3 hours at 22° C. After ligation, RNA samples from both total RNA and RPFs were reverse transcribed using SuperScript III Reverse Transciptase (Thermo Fisher Scientific) and 0.25 μM RT primer (IDT). cDNA samples were then gel-extracted on 10% polyacrylamide/TBE/Urea gels (Thermo Fisher Scientific) and circularized by CircLigase ssDNA Ligase (Lucigen) for 2 hours at 60° C. cDNA libraries for total RNA and RPFs were amplified for 9 and 12 PCR cycles, respectively, using Phusion High-Fidelity PCR Master Mix (NEB), Illumina index primers and 10 μM forward primer (IDT). Amplified libraries were cleaned using AMPure XP Beads (Beckman Coulter), followed by gel-extraction on 8% native TBE gels (Thermo Fisher Scientific). Libraries were sequenced with Illumina NextSeq 500.

### Ribo-Seq Data Analysis

The sequences of input and Ribo-seq samples were firstly processed to get the clean reads by trimming the adapters and filtering the low-quality sequences. Then, for Ribo-seq input data, the clean reads were aligned to reference genome sequences using STAR(Dobin et al., 2013). The resulted BAM mapping files were used as inputs of HTSeq(Anders et al., 2015) to calculate the read counts for each gene from GENCODE gene mode. For the cleaned Ribo-seq data, the clean ribosome-protected fragments (RPFs) were firstly collapsed into FASTA format by fg2collapedFa. RiboToolkit (https://bioinformatics.caf.ac.cn/RiboToolkit_demo) was used to perform codon occupancy analysis and translation efficiency analysis by uploading the collapsed RPF tags and gene read counts from input samples (Liu et al., 2020a). In brief, rRNA and tRNA sequences were filtered from RPF containing files by alignment to rRNA sequences (Ensembl non-coding, release 91)(Zerbino, et al., 2018) and tRNA sequences from GtRNAdb databases (Chan and Lowe, 2016). The resulting ribosome-protected fragments (RPFs) were aligned to the mouse reference genome (mm10) using STAR [10.1093/bioinformatics/bts635] and only unique mapped reads were kept. The genome unique mapping reads were then mapped to transcript sequences using Bowtie with a maximum of one mismatch allowed (Langmead et al., 2009). and all the transcript mappings were kept. CONCUR tool (https://github.com/susbo/concur) was used for calculating codon usage based on the reference genome mapping based on STAR (Frye et al., 2020). The translation efficiency (TE) was calculated by dividing RPF abundance on CDS by its mRNA abundance of input sample. A threshold of two-fold change and FDR<0.05 was used to define the differential translation genes (Zinshteyn and Gilbert, 2013). PausePred (https://pausepred.ucc.ie/), was used to infer ribosome pauses from Ribo-seq data. Peaks of ribosome footprint density are scored based on their magnitude relative to the background density within the surrounding area (Kumari et al., 2018).

### Proteomic Analysis by Stable Isotope Labeling Using Amino Acids in Cell Culture (SILAC)

LNZ308 human glioblastoma cells or LPS853 liposarcoma cells were grown in media supplemented with isotopic-labeled 13C615N2 1-lysine and 13C615N4 1-arginine (heavy) or normal amino acids (light) for 15 to 21 days until a labeling efficiency >95% was achieved following the instructions of the SILAC Protein Quantitation Kit (Trypsin)—DMEM (A33972, Thermo Scientific). Cells were lysed in 1× passive cell lysis buffer (Promega) supplemented with cOmplete protease inhibitor (11873580001, Roche). Protein lysates were cleared by centrifugation at 14,000×g for 5 minutes at 4° C. Equal amounts of heavy and light protein (1:1) amounts were mixed with 2× reducing sample buffer an 100 μg of clarified sample was separated by SDS-PAGE (4-20%). Three technical replicates were performed for each sample. Gels were stained using Novex Colloidal blue staining (Invitrogen) and each lane was cut into 12 slices of equal size. Excised gel bands were cut into approximately 1 mm3 pieces. The samples were reduced with 1 mM DTT for 30 minutes at 60° C. and then alkylated with 5 mM iodoacetamide for 15 minutes in the dark at room temperature. Gel pieces were then subjected to a modified in-gel trypsin digestion procedure (Shevchenko, et al., 1996, Anal Chem.). Gel pieces were washed and dehydrated with acetonitrile for 10 min. followed by removal of acetonitrile. Pieces were then completely dried in a speed-vac. Rehydration of the gel pieces was with 50 mM ammonium bicarbonate solution containing 12.5 ng/μl modified sequencing-grade trypsin (Promega, Madison, WI) at 4° C. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (˜1 hr). The samples were then stored at 4° C. until analysis. On the day of analysis the samples were reconstituted in 5-10 μl of HPLC solvent A (2.5% acetonitrile, 0.1% formic acid). A nano-scale reverse-phase HPLC capillary column was created by packing 2.6 pm C18 spherical silica beads into a fused silica capillary (100 pm inner diameter×˜30 cm length) with a flame-drawn tip (Peng and Gygi J). After equilibrating the column each sample was loaded via a Famos auto sampler (LC Packings, San Francisco CA) onto the column. A gradient was formed and peptides were eluted with increasing concentrations of solvent B (97.5% acetonitrile, 0.1% formic acid). As each peptide was eluted they were subjected to electrospray ionization and then they entered into an LTQ Orbitrap Velos Pro ion-trap mass spectrometer (Thermo Fisher Scientific, San Jose, CA). Eluting peptides were detected, isolated, and fragmented to produce a tandem mass spectrum of specific fragment ions for each peptide. Peptide sequences (and hence protein identity) were determined by matching protein or translated nucleotide databases with the acquired fragmentation pattern by the software program, Sequest (ThermoFinnigan, San Jose, CA) (Eng. et al., 1994 J. Am. Soc. Mass. Spectrom). The differential modification of 8.0142 and 10.0083 mass units for lysine and arginine, respectively were included in the database searches to find SILAC labeled peptides. All databases include a reversed version of all the sequences and the data was filtered to between a one percent or lower peptide false discovery rate. SILAC protein ratios (H/L) were determined as the average of all peptide ratios assigned to a protein between heavy and light samples. Differential protein expression was determined using a moderated t-test, testing for the null-hypothesis being no change in H/L ratio. Multiple tests were corrected using false discovery rate (<1%).

### Animal Studies

4-6 weeks old female NU/J (Nude) immunodeficient mice (Jackson Laboratory #002019) were used for subcutaneous injections. LNZ308 or LPS853 cells (5×10⁶ cells) shGFP or shARG were transplanted into nude mice. The indicated number of cells were mixed with serum-free medium and growth factor reduced Matrigel (Corning #354230) (1:1) and injected into the right flank of nude mice. Five or six mice were used for each group. Subcutaneous tumor formation was monitored by calipers twice a week. The tumor volume was calculated using the formula 1/2(length×width). The recipient mice were monitored and euthanized when the tumors reached 1 cm in diameter. At end-point tumors were collected and weighted. Randomization and blinding were not applied.

1. A method for treating cancer, the method comprising administering a composition comprising an inhibitor of an oncogenic transfer RNA (tRNA) to a subject in need thereof,
wherein the oncogenic tRNA comprises ARG-TCT-4-1, thereby treating cancer in the subject.

2. The method of claim 1, wherein the inhibitor reduces expression and/or activity of the oncogenic tRNA.

3. The method of claim 1, wherein the inhibitor sequesters the oncogenic tRNA, thereby reducing activity of the oncogenic tRNA.

4. The method of claim 1, wherein the inhibitor comprises an inhibitory nucleic acid.

5. The method of claim 4, wherein the inhibitory nucleic acid comprises a targeting sequence complementary to a sequence of ARG-TCT-4-1 of about 15-25 nucleotides in length.

6. The method of claim 5, wherein the targeting sequence comprises SEQ ID NO: 1, or SEQ ID NO: 2.

7. The method of claim 4, wherein the inhibitory nucleic acid is about 25-65 nucleotides in length.

8. The method of claim 4, wherein the inhibitory nucleic acid comprises an siRNA, an shRNA, an miRNA, or an antisense oligonucleotide.

9. The method of claim 8, wherein the inhibitory nucleic acid is an shRNA and comprises a sequence of SEQ ID NO: 3 or SEQ ID NO: 4.

10. The method of claim 4, wherein the inhibitory nucleic acid is an antisense oligonucleotide and comprises a sequence of SEQ ID NOs: 5, 6 or 7.

11. The method of claim 4, wherein the inhibitory nucleic acid comprises a nucleic acid modification.

12. The method of claim 11, wherein the nucleic acid modification comprises a locked nucleic acid, a phosphorothioate modification, a 2′-O-methyl modification, a 2′-O-methoxyethyl modification, a 2′-fluoro modification, a phosphorodiamidate modification or a mesylphosphoramidate modification.

13. The method of claim 4, wherein the inhibitory nucleic acid specifically binds ARG-TCT-4-1.

14. The method of claim 13, wherein the inhibitory nucleic acid binds ARG-TCT-4-1 at a concentration that is at least 2-fold lower than the concentration of the inhibitory nucleic acid that binds an off-target tRNA.

15. The method of claim 14, wherein the inhibitory nucleic acid does not bind to off-target tRNA.

16. The method of claim 1, wherein the cancer comprises increased expression of ARG-TCT-4-1 and/or methyltransferase-like 1 protein (METTL1).

17. The method of claim 1, wherein the cancer is a sarcoma, a glioblastoma, an adrenocortical carcinoma, a cholangiocarcinoma, a melanoma, a glioma, a diffuse glioma, a mature B cell neoplasm, a non-small cell lung cancer, an esophagogastric adenocarcinoma, a pheochromocytoma, a hepatocellular carcinoma, an endometrial carcinoma, a pancreatic adenocarcinoma, a breast carcinoma, an invasive breast carcinoma, a head and neck squamous cell carcinoma, a bladder urothelial carcinoma, a colorectal adenocarcinoma, an ovarian epithelial tumor, a prostate adenocarcinoma, a cervical squamous cell carcinoma, a renal non-clear cell carcinoma, or a renal clear cell carcinoma.

18. The method of claim 17, wherein the sarcoma is a liposarcoma.

19. The method of claim 1, wherein the composition further comprises a lipid composition or a lipid nanoparticle.

20. The method of claim 19, wherein the inhibitory nucleic acid, the lipid composition or the lipid nanoparticle further comprises a targeting moiety.

21. The method of claim 20, wherein the targeting moiety comprises an antigen or antigen-binding fragment thereof that binds to a cancer cell marker, or wherein the targeting moiety comprises a ligand.

22.-54. (canceled)
